SEQUENCE LISTING (86 sequences)

SEQ ID NO: 1 - protein, 287 aa, Artificial Sequence
  Complete sequence of the CBP protein. Amino Acid Nos 1-16 is the signal peptide
MKAVLLLALLGAFTNAAPLLSNQRLGSAEEEKFCSTHQGEVHARFWLQMRVGVRHSPLYTPSNMCMMDIEDSTDTEDSTMEKEYTSTATGDADGLNVSVALIGEGVSIPLSYIGLRFNPSLTDGYLYVNVSSRAPWDQQTLDLSANDGWGIKQVLEKEILAIQIGCDNQKFPEEPTTTQPPSPVTTTLSSTTLDPNDENTDTTPTTTGDSVDGKRNPDDFDFSLIVDPRCVTSVNLHFEIKDACMDHKESSPLSLKGEYGDGELVRKEIKNVGKDHNMCSLNLSPGH

SEQ ID NO: 2 - protein, 271 aa, Artificial Sequence
  Mature sequence of the CBP protein
APLLSNQRLGSAEEEKFCSTHQGEVHARFWLQMRVGVRHSPLYTPSNMCMMDIEDSTDTEDSTMEKEYTSTATGDADGLNVSVALIGEGVSIPLSYIGLRFNPSLTDGYLYVNVSSRAPWDQQTLDLSANDGWGIKQVLEKEILAIQIGCDNQKFPEEPTTTQPPSPVTTTLSSTTLDPNDENTDTTPTTTGDSVDGKRNPDDFDFSLIVDPRCVTSVNLHFEIKDACMDHKESSPLSLKGEYGDGELVRKEIKNVGKDHNMCSLNLSPGH

SEQ ID NO: 3 - DNA, 1162 bp, Artificial Sequence
  Complete sequence of the ORFV112 gene
ttgctgctgctatgtcgtgcccgactctgtgcgacaaagaagcagcttaccagacgactcttcgttcatgttctctaagcaagaacactggagtgagttgccatttccgtctccaaccatataattagcatccttgtttttatcctgtatttttatcagtttttagctagttaaaacataaatagtaaagctaaaaagaggagttctggaatcttgcaacgacaggatgaaggcggtgttgttgctagcgctactgggagcgttcaccaacgcagcgcctttgttaagcaaccagcgtcttggcagtgcagaagaagaaaaattctgctcgacgcatcagggcgaagtgcacgccaggttctggcttcagatgcgcgtgggtgtacgacacagtccgctctacactcccagcaacatgtgcatgatggacatagaagactctacggacacagaagactccacgatggaaaaagaatacacgtctacggcaacgggtgatgcggacggattgaacgtgtccgtagcactaattggagaaggcgtgagcataccgctaagttacataggccttagatttaacccgtcgcttacagatggctacctgtacgtcaacgtctcgtcacgggctccttgggatcaacagactctggacctatccgcgaacgacggctggggtatcaaacaggttctagaaaaagagatactggccatccagatagggtgcgacaaccaaaaatttcccgaagaacccacaactacccaacccccctcacctgtcacaacaacgctttcctcaacaactctagatccgaatgacgaaaacacagacactacgccgacaaccaccggcgacagtgtagacggaaagcgcaatccagatgactttgacttctcgctgatcgtggacccccgatgcgtgacctctgtaaacctgcactttgagattaaggacgcgtgcatggaccacaaagaatcgtcgccgttgtcgctgaagggggaatatggagacggcgaactagtaagaaaagaaatcaaaaacgtgggaaaggatcacaatatgtgcagtcttaacctcagccctggccattgagctgtttttattcggcaatataataggtgattattgaacattaaacaaaacttatcccacaacgccgcaaca

SEQ ID NO: 4 - DNA, 624 bp, Artificial Sequence
  5′ end partially deleted ORFV112 gene
ggcgtgagcataccgctaagttacataggccttagatttaacccgtcgcttacagatggctacctgtacgtcaacgtctcgtcacgggctccttgggatcaacagactctggacctatccgcgaacgacggctggggtatcaaacaggttctagaaaaagagatactggccatccagatagggtgcgacaaccaaaaatttcccgaagaacccacaactacccaacccccctcacctgtcacaacaacgctttcctcaacaactctagatccgaatgacgaaaacacagacactacgccgacaaccaccggcgacagtgtagacggaaagcgcaatccagatgactttgacttctcgctgatcgtggacccccgatgcgtgacctctgtaaacctgcactttgagattaaggacgcgtgcatggaccacaaagaatcgtcgccgttgtcgctgaagggggaatatggagacggcgaactagtaagaaaagaaatcaaaaacgtgggaaaggatcacaatatgtgcagtcttaacctcagccctggccattgagctgtttttattcggcaatataataggtgattattgaacattaaacaaaacttatcccacaacgccgcaaca

SEQ ID NO: 5 - DNA, 20 bp, Artificial Sequence
  ORFV112 gene-specific forward primer
catgcccaactgctacttcg

SEQ ID NO: 6 - DNA, 20 bp, Artificial Sequence
  ORFV112 gene-specific reverse primer
acctgtttgataccccagcc

SEQ ID NO: 7 - DNA, 42 bp, Artificial Sequence
  Forward primer for amplification of the ORFV112 gene′s left arm
gaccatgattacgccaagcttcgtgatttgggaatatgcacc

SEQ ID NO: 8 - DNA, 76 bp, Artificial Sequence
  Reverse primer for amplification of theORFV112 gene′s left arm
aaaataaaatttcaatttttataacttcgtatagcatacattatacgaagttatgcagcattaactgcgcgctggg

SEQ ID NO: 9 - DNA, 55 bp, Artificial Sequence
  Forward primer for amplification of the ORFV112 gene′s right arm
ttttttttggaatataaatagctagcttaattaacttatcccacaacgccgcaac

SEQ ID NO: 10 - DNA, 41 bp, Artificial Sequence
  Reverse primer for amplification of the ORFV112 gene′s right arm
aaaacgacggccagtgaattcgtatctatcgtcgggaccag

SEQ ID NO: 11 - DNA, 65 bp, Artificial Sequence
  Forward primer for amplification of the EGFP gene
aaaaattgaaattttattttttttttttggaatataaataaccggtcgccaccatggtgagcaag

SEQ ID NO: 12 - DNA, 100 bp, Artificial Sequence
  Reverse primer for amplification of the EGFP gene
tatttatattccaaaaaaaaaaaataaaatttcaatttttataacttcgtatagcatacattatacgaagttatacgcgttaagatacattgatgagttt

SEQ ID NO: 13 - DNA, 42 bp, Artificial Sequence; identical to SEQ ID NO: 7
  Forward primer for amplification of the linear CBP shuttle vector

SEQ ID NO: 14 - DNA, 41 bp, Artificial Sequence; identical to SEQ ID NO: 10
  Reverse primer for amplification of the linear CBP shuttle vector

SEQ ID NO: 15 - DNA, 30 bp, Artificial Sequence
  Forward primer for amplification of the px330 backbone
gtaagaattcctagagctcgctgatcagcc

SEQ ID NO: 16 - DNA, 27 bp, Artificial Sequence
  Reverse primer for amplification of the px330 backbone
tggcaccggtccaacctgaaaaaaagt

SEQ ID NO: 17 - DNA, 29 bp, Artificial Sequence
  Forward primer for amplification of Cas9 without the NLS (sequence)
ttggaccggtgccaccatggacaagaagt

SEQ ID NO: 18 - DNA, 30 bp, Artificial Sequence
  Reverse primer for amplification of Cas9 without the NLS (sequence)
ggctgatcagcgagctctaggaattcttac

SEQ ID NO: 19 - DNA, 20 bp, Artificial Sequence
  PCR identification primer 1 (OVM38F/R)-Forward primer
gagacgccgccgagcaactt

SEQ ID NO: 20 - DNA, 21 bp, Artificial Sequence
  PCR identification primer 1 (OVM38F/R)-Reverse primer
tgcagcacttcctggacatcg

SEQ ID NO: 21 - DNA, 19 bp, Artificial Sequence
  PCR identification primer 2 (OVM43F/R)-Forward primer
ggacgatcctgtgcggtag

SEQ ID NO: 22 - DNA, 20 bp, Artificial Sequence
  PCR identification primer 2 (OVM43F/R)-Reverse primer
ttgtcggcgtagtgtctgtg

SEQ ID NO: 23 - DNA, 23 bp, Artificial Sequence
  PCR identification primer 3 (OVM44F/R)-Forward primer
gatgactttgacttctcgctgat

SEQ ID NO: 24 - DNA, 20 bp, Artificial Sequence
  PCR identification primer 3 (OVM44F/R)-Reverse primer
cgacagatccatttcccaat

SEQ ID NO: 25 - DNA, 19 bp, Artificial Sequence
  PCR identification primer 4 (OVM45F/R)-Forward primer
tgcccaactgctacttcgc

SEQ ID NO: 26 - DNA, 20 bp, Artificial Sequence
  PCR identification primer 4 (OVM45F/R)-Reverse primer
caccttgatgccgttcttct

SEQ ID NO: 27 - DNA, 22 bp, Artificial Sequence
  PCR identification primer 5 (OVM46F/R)-Forward primer
agccataccacatttgtagagg

SEQ ID NO: 28 - DNA, 20 bp, Artificial Sequence
  PCR identification primer 5 (OVM46F/R)-Reverse primer
agacgatcaccagcacttcc

SEQ ID NO: 29 - DNA, 21 bp, Artificial Sequence
  PCR identification primer 6 (OVM3F2/4R2)- Forward primer
cgtgatttgggaatatgcacc

SEQ ID NO: 30 - DNA, 22 bp, Artificial Sequence
  PCR identification primer 6 (OVM3F2/4R2)- Reverse primer
gcagcattaactgcgcgctggg

SEQ ID NO: 31 - protein, 281 aa, Artificial Sequence
  Orf virus strain YX AKU76602.1
MKVVLLLVLLGALTNAAPVGNQRIDSEEKANFCSTHQNEVYARFRLQMRVGVRHSPLYVPSNMCMMDIEDSVMDNEYMSAATGDADGVNVSVALIGEGVSIPLSYIGLGFNPSLADGYLYVNVSSRAPWVQQTQDLSANGSWGIKQVLEQEMLAIQIGCDNQKFPEEPTTTQPPSPVTTTLSSTTLDSNDENADTTPPTTTSASVNRKRNSDDFDFSLLVDPRCVTSVDLHVELRDACIDYKEASLLSLKGKYGDGELVKKEIKDVGKDHNMCSLNLSPGH

SEQ ID NO: 32 - protein, 286 aa, Artificial Sequence
  Orf virus strain NA1/11AHZ33810.1
MKAVLLLALLGAFTNAAPLLESQRSNSEEKANFCSTHNNEVYARFRLQMRVGVRHSPFYTPSNMCMMDIEDSVEDIEESTEKEYASTATGEAAGVNVSVALVGEGVSIPFSYIGLGFNPSLEDSYLYVNVSSRAPWVKQTSDLSANGGWGIKQVLEKELLAIQIGCDNQKFPEEPTTTPPSPVTTTLSSTTPDLNEENTNTTPTTTSASVDRRRNLDDIDFSLLVDPRCVTSVDLHVELRDACMDYKQESPLSLKGKYGDGELVKKEIKDVGKNYNMCSLNLNPGN

SEQ ID NO: 33 - protein, 286 aa, Artificial Sequence
  Orf virus strain NZ2 ABA00630.1
MKAVLLLALLGAFTNAAPLLESQRSNSEEKANFCSTHNDEVYARFRLQMRVGVRHSPLYTPSNMCMLDIEDSVEDIEESTEKEYASTATGEAAGVNVSVALVGEGVSIPFSYIGLGFNPSLEDSYLYVNVSSRAPWVKQTSDLSANGGWGIKQVLEKELLAIQIGCDNQKFPEEPTTTPPSPVTTTLSSTTPDLNEENTENTPTTTGASVDRKRNPADDFSLLVDPRCVTSVDLHVELRDAICIDYKQESPLSLKGKYGDGELVKKEIKDVGKNHNMCSLNLNPGN

SEQ ID NO: 34 - protein, 289 aa, Artificial Sequence
  Orf virus strain NA17 AYM26053.1
MKVVVLLALLGALTNAAPVGNQRLNSKEKEDFCSTHQNEVYARFRLQMRVGVRHSPLYVPSNMCMMDIEDSVDDIEEDSIIVKEFTSTATGEAAGVNVSVALVGEGVSIPFSYIGLGFNPSLEDSYLYVNVSSRAPWVQQTPDLSANDSWGIKQVLEKELLAIQIGCDNQKFPEEPTTTQPPSLVTTTLSSTTLDLNDENTDTTPPTTTSASVNKKRNPDDFDFSLLVDPRCVTSVDLHVELRDACIDYKETSQLSLKGEYGDGELIKKEIKDVGKDHNMCSLNLNPGN

SEQ ID NO: 35 - protein, 288 aa, Artificial Sequence
  Orf virus strain OV-SA00 AAR98337.1
MKVVLLLVLLGAFTNAAPLVGNQRLDSEEKANFCSTHQNEVYARFRLQMRVGVRHSPLYTPSNMCMMDIEDSIMDIENSTEKEYTSAATGDANGVNVSVALIGEGVSIPLSYIGLGFNPSLADGYLYVNVSSRAPWVQQTLDLSANGGWGINQILEKELLAIQIGCDNQKFPEEPTTTQPPSPVTTTLSSTTLDLTDENTDTTPLTTTSASVNRKRNPDDFDFSLLVDPRCVTSVDLHVELRDACIDYKEASQLSLKGKYGDGELVKKEIKDVGKDHNMCSLNLSPGH

SEQ ID NO: 36 - protein, 286 aa, Artificial Sequence
  Orf virus strain OV-IA82 AAR98207.1
MKAAAVLLLALLGAFTNAAPVSNQRLGSEEKEKFCSTHHDEVYARFRLQMRVGVRHSPLYVPSNMCMMDIEDSTDDIEESTEKEYTSTATGEAAGVNVSVALVGEGVKIPFSYIGLGFNPSTDGYLYVNVSSRAPWVQQTPDLSANSGWGIKQVLEKELLAIQIGCDNQKFPEEPTTTPSLVTTTLSPTTTLNPNNENTDTTPTPTGASVDGKRNPDDIDFSLIVDPRCVTSVNLHFELKDACMDYKKESPLSLKGKYGDSELVKQEIKDVGKNHNMCSLNLSPGH

SEQ ID NO: 37 - protein, 280 aa, Artificial Sequence
  Orf virus strain GO AKU76734.1
MKVVLLLVLLGALTNAAPVGNQRIDSEEKANFCSTHQNEVYARFRLQMRVGVRHSPLYTPSNMCMMNIEDSMMDNEYTSTATGDADGVNVSVALMGKGVSIPLSYIGLGFNPLLADGYLYVNVSSRAPWVQQTPDLSANGGWGIKQVLEEEILAIQIGCDNQKFLEEPTTTQPPSLVTTTLSSTTLGLTDENTDTTPTTTSASVDRKRNLDDIDFSLLVDTRCVTSVNLHFEIKDACMDYKKESPLMMKGGYGDGELVRKEIKYVGTNHTMCSLNLSPGY

SEQ ID NO: 38 - protein, 280 aa, Artificial Sequence
  Orf virus strain SJ1 AKU76990.1
MKVVVLLALLGALTNAAPVGNQRLNSKEKEDFCLTHPDEVYARFRLHMRVGVRHSPLYTPSNMCMMNIEDSMMDNEYTSTATGDADGVNVSVALMGKGVSIPLSYIGLGFNPLLADGYLYVNVSSRAPWVQQTPDLSANGGWGIKQVLEEEILAIQIGCDNQKFLEEPTTPQPPSLVTTTLSSTTLGLTDENTDTTPTTTSASVDRKRNLDDIDFSLLVDTRCVTSVNLHFEIKDACMDYKKESPLMMKGGYGDGELVRKEIKYVGTNHTMCSLNLSPGY

SEQ ID NO: 39 - protein, 287 aa, Artificial Sequence
  Orf virus strain SY17 AYN61060.1
MKAVLLLALLGAFTNAAPVGNQRLDSGEKEKFCSTHQDEVYARFRLQMRVGVRHSPFYTPSNMCMMDIEDSVDDIEEDSIIVKEFTSTATGEADGVNVSVALVGEDVKIPLSYIGLGFNPSTDGYLYVNVSSRAPWVQQTLDLSANDSWGIKQVLEKELLAIQIGCDNQKFPEEPTTTQPPSLVTTTLSSTTPDLNDENTDTTPTTTGASVDRKRNPADFSFSLLVDPRCVTSVDLHVELRDACMEYKETSPLSLKGEYGDGELVKKEIKDVGKNHNMCSLNLNPGN

SEQ ID NO: 40 - protein, 286 aa, Artificial Sequence
  Orf virus strain OV-HN3/12 ASY92409.1
MKAVLLLALLGAFTNAAPLLESQRSNSEEKANFCSTHNNEVYARFRLQMRVGVRHSPFYTPSNMCMMDIEDSVEDIEESTEKEYASTATGEAAGVNVSVALVGEGVSIPFSYIGLGFNPSLEDSYLYVNVSSRAPWVKQTSDLSANGGWGIKQVLEKELLAIQIGCDNQKFPEEPTTTPPSPVTTTLSSTTPDLNEENTDTTPTTTSASVDRRRNLDDIDFSLLVDPRCVTSVDLHVELRDACMDYKQESPLSLKGKYGDGELVKKEIKDVGKNYNMCSLNLNPGN

SEQ ID NO: 41 - protein, 289 aa, Artificial Sequence
  Orf virus strain NP AKU76866.1
MKVVVLLALLGALTNAAPVGNQRLNSKEKEDFCSTHPDEVYARFRLQMRVGVRHSPLYVPSNMCMMDIEDSVDDIEEDSIIVKEFTSTATGEAAGVNVSVALVGEGVSIPFSYIGLGFNPSLEDSYLYVNVSSRAPWVQQTPDLSANDSWGIKQVLEKELLAIQIGCDNQKFPEEPTTTQPPSLVTTTLSSTTLDLNDENTDTTPPTTTSASVNKKRNPDDFDFSLLVDPRCVTSVDLHVELRDACIDYKETSQLSLKGEYGDGELIKKEIKDVGKDHNMCSLNLNPGN

SEQ ID NO: 42 - protein, 295 aa, Artificial Sequence
  Orf virus strain B029 AHH34297.1
MKAVLLLALLGAFTNAAPLLSNQRLGSAEEEKFCSTHHDKVYARFWLQMRVGVRHSSLYAPSNMCMMDIEDSTVDIEGSTETEDSTVEKEYTSAATGDANGVNVSVALMGEGVSIPLSYIGLGFNPLLKDGYLYVNVSSRAPWDQQTLDLSANDGWGIKQVLEKEILAIQIGCDNQKFPEEPTTTQPPSPVTTTLSPTTTLNPNNENTDTTPTPTGASVDGKRNPDDIDFSLIVDPRCVTSVNLHFEIKDACMDYKQESPLSLKGKYGDGELVKEEIKDVGKNHNMCSLNLSPGH

SEQ ID NO: 43 - DNA, 1069 bp, Artificial Sequence
  Orf virus strain YX KP010353.1
ttgctgctgctatatcgtacctgactctgtgttttaaagacagcagctcaccgactcttcgtccctgttctccaagcaagaacactggagtgatttgccatttccgtctccaacatataattagcatccttgtttttatcttgtctttttatcagtttttatgttagttaaaacataaatagtaaagctaaaaagagtactctggaatcttgcaacagtcaggatgaaggtggtgttgttgctagtgctactgggggcgttaaccaacgcagcgcccgtcggcaaccagcgtattgacagtgaggagaaagcaaatttctgctcgacgcatcaaaatgaagtgtacgccaggttccggcttcagatgcgcgtgggtgtacgacacagtccgctctacgttcccagcaacatgtgcatgatggacatagaagactccgtaatggataatgaatacatgtctgcggccacgggtgatgcggacggagtgaacgtgtccgtggcactaataggagaaggcgtgagcataccgctaagttacataggcctcggatttaacccatcgcttgcagatggctacctgtacgtcaacgtctcgtcacgggctccttgggttcaacagactcaagacttatctgcgaacggcagctggggtattaaacaggttctagaacaagagatgctggccatccagatagggtgcgacaaccaaaaatttcctgaagaacccacaactacccaacctccctcacctgtcacgacaacgctttcctcaacaactctagattcgaatgacgaaaacgcagacactacgccgccgaccaccaccagcgccagtgtaaacagaaagcgcaattcagatgactttgacttctcgctgctcgtagacccccgatgcgtgacctctgtagacctgcacgtcgagctcagggacgcgtgcatagactacaaagaagcgtcgctgttgtcgctgaaggggaaatatggagacggcgaactagtaaaaaaggagatcaaagacgtgggaaaggatcacaatatgtgcagtcttaacctcagccctggccattga

SEQ ID NO: 44 - DNA, 1036 bp, Artificial Sequence
  Orf virus strain NA1/11 KF234407.1
tcaacatccttgtttttatcctgtctttttatcagttttttagctagttaaaacataaatagtaaagctaaaaagaggagttctggagtcttgcaataaccaggatgaaggcggtgttgttgctggcgttactgggagcgttcaccaacgcagcgcctttgttagaaagccagcgttctaacagtgaggagaaagcaaatttctgctcgacgcataataatgaagtgtacgccaggttcaggcttcagatgcgcgtgggtgtacgacacagtcctttctacactcccagcaacatgtgcatgatggacatagaagactccgttgaggacatagaagagtccacagagaaagaatacgcgtctacggccacgggtgaggcggccggagtgaacgtgtcagtggcactagtgggagaaggcgtgagcataccgtttagttacataggccttggattcaacccgtcgcttgaagatagctacctgtacgttaacgtctcgtcacgagctccttgggttaaacagacttcggacctatccgcgaacggcggctggggtattaaacaggttctagaaaaagagttactggccatccaaatagggtgcgacaaccaaaaatttcccgaagaacccacaactacacccccctcacctgtcacgacaacgctttcctcaacaactccagatctgaatgaagaaaacacaaacactacgccgacgaccaccagcgccagtgtagacagaaggcgcaatctagatgacattgacttctcgctgctcgtggacccccgatgcgtgacctctgtagacctacacgtcgagctcagggacgcgtgcatggactacaaacaagagtcgccgttgtcgctgaaggggaaatatggagacggcgagctagtaaaaaaggagatcaaagacgtgggaaagaattacaatatgtgcagtcttaacctcaaccctggcaattgagatgtttttattcggcaatataatagggattattgaacattaaacaaaacttatcccacaacgccgcaaca

SEQ ID NO: 45 - DNA, 1036 bp, Artificial Sequence
  Orf virus strain NZ2 DQ184476.1
tcagcatccttgtttttatcctgtctttttatcagttttttagctagttaaaacataaatagtaaagctaaaaagaggagttctggagtcttgcaacaaccaggatgaaggcggtgttgttgctggcgttactgggagcgttcaccaacgcagcgcctttgttagaaagccagcgttctaacagtgaggaaaaagcaaatttctgctcgacgcataatgatgaagtgtacgccaggttcaggcttcagatgcgcgtgggtgtacgacacagtccgctctacactcccagcaacatgtgcatgctggacatagaagactccgttgaggacatagaagagtccacagaaaaagaatacgcgtctacggccacgggtgaggcggccggagtgaacgtgtcagtggcactagtgggagaaggcgtgagcataccgtttagttacataggccttggattcaacccgtcgcttgaagatagctacctgtacgtcaacgtctcgtcacgagctccttgggttaaacagacttcggacctatccgcgaacggcggctggggtatcaaacaggttctagaaaaagagttactggccatccaaatagggtgcgacaaccaaaaatttcccgaagaacccacaactacacccccctcacctgtcactacaacgctttcctcaacaactccagatctgaatgaagaaaacacagaaaatacgccgacgaccaccggcgccagtgtagacagaaagcgcaatccagctgacattgacttctcgctgctcgtggacccccgatgcgtgacctctgtagacctgcacgtcgagctcagggacgcgtgcatagactacaaacaagagtcgccgttgtcgctgaaggggaaatatggagacggcgaactagtaaaaaaggagattaaagacgtgggaaagaatcacaatatgtgcagtcttaacctcaaccctggcaattgagctgtttttattcggcaatataataggtgattattgaacattaaacaaaacttatcccacaacgccgcaac

SEQ ID NO: 46 - DNA, 1166 bp, Artificial Sequence
  Orf virus strain NA17 MG674916.2
ttgctgctgctatatcgtacctgactctgtgttacaaagacagcagctcaccagactcttcgtccctgttctctaagcaagaacactggagtgagttgccatttccgtctccaacatataattagcatccttgtttttatcttgtctttttatcagtttttatgctagttaaaacataaatagtaaagctaaaaagagtactctggaatcttgcaacaatcaggatgaaggtggtggtgttgctggcgctactgggagcgttaaccaacgcagcgcccgtcggcaaccagcgtcttaacagtaaagagaaagaagatttctgctcgacgcatcaaaatgaagtgtacgccaggttccggcttcagatgcgcgtgggtgtacgacacagtccgctctacgttcccagcaacatgtgcatgatggacatagaagactctgttgatgacatagaagaagactccattatagtaaaagaattcacgtctacagccacgggtgaggcggccggagtgaacgtgtccgtggcactagtgggagaaggcgtgagcataccgtttagttacataggccttggattcaacccgtcgcttgaagatagctacttgtacgtcaacgtctcgtcacgagctccttgggttcaacagactccagacttatctgcaaacgacagctggggtattaaacaggttctagaaaaagagttactggccatccaaatagggtgcgacaaccaaaaatttcccgaagaacccacaaccacccaacccccctcacttgtcacgacaacgctttcctcaacaactctagatctgaatgacgaaaacacagacactacgccgccaaccaccaccagcgccagcgtaaacaaaaagcgcaatccagatgactttgacttctcgctgctcgtggacccccgatgcgtgacctctgtagacctgcacgtcgagctcagggacgcgtgcatagactacaaagaaacgtcgcagttgtcgctgaagggggaatatggagacggcgaactaataaaaaaggagatcaaagacgtgggaaaggatcacaatatgtgcagtcttaacctcaaccctggcaattgagctgtttttattcggcaatataataggtgattattgaacattaaacaaaacttatcccacaacgccgcaaca

SEQ ID NO: 47 - DNA, 1161 bp, Artificial Sequence
  Orf virus strain OV-SA00 AY386264.1
ttgctgcgctatgtcgtgcccgactctgtgcgacaaagacagaagctcaccaaactcttcgtctctgttctccaagcaagaacactggagtgatttgccatttccgtctccaacatataattagcatccttgtttttatcttgtctttttatcagtttttatgttagttaaaacataaatagtaaagctaaaaagagtactctggaatcttgcaacagtcaggatgaaggtggtgttgttgctagtgctactgggagcgtttaccaacgcagcgcctttggtcggcaaccagcgtcttgacagtgaggagaaagcaaatttctgctcgacacatcaaaatgaagtgtacgccaggttccggcttcagatgcgcgtgggtgtacgacacagtccgctctacactcccagcaacatgtgcatgatggacatagaagactccattatggacatagaaaactccacagaaaaagaatacacgtctgcagccacgggtgatgcgaacggagtgaacgtgtccgtggcactaataggagaaggcgtgagcataccgctaagttacataggcctcggattcaacccatcgcttgcagatggctacctgtacgtcaacgtctcgtcacgagctccttgggttcaacagactctagacctatccgcgaacggcggctggggtattaatcagattctagaaaaagagttactggccatccaaatagggtgcgacaaccaaaaatttcccgaagaacccacaactacccaacctccctcacctgtcacgacaacgctttcctcaacaacgctagatctgactgacgaaaacacagacactacgccgctgaccaccaccagcgccagtgtaaacagaaagcgcaatccagatgactttgacttctcgctgctcgtggacccccgatgcgtgacctctgtagacttgcacgtcgagctcagggacgcgtgcatagactacaaagaagcgtcgcagttgtcgctgaaggggaaatatggagacggcgaactagtaaaaaaggagatcaaagacgtgggaaaggatcacaatatgtgcagtcttaacctcagccctggccattgagatgtttttattcggcaattaataggtgattattgaacattaaacaaaacttatcccacaacgccgcaaca

SEQ ID NO: 48 - DNA, 1159 bp, Artificial Sequence
  Orf virus strain OV-IA82 AY386263.1
ttgctgctgctatgtcgtgcccgactctgtgcgacaaagacagcggctaaccagactcttcgtccctgttctccaagcaaaaaactggagtgagttgccatttccgtctccaaccatataattagcatccttgtttttatcctgtatttttatcagtttttatgctagttaaaacataaatagtaaggctaaaaagaagagttctagaatcttgcaacaaccaagatgaaggcggcggcggtgttgttgctagcgctactgggagcgttcaccaacgcagcgcccgtcagcaaccagcgtcttggcagtgaggagaaagaaaaattctgctcgactcatcatgacgaagtgtacgccaggttccggcttcagatgcgcgtgggtgtacgacacagtccgctctacgttcccagcaacatgtgcatgatggacatagaagactctacggatgacatagaagagtccacggagaaagaatacacgtctacggctacgggtgaggcggccggagtgaacgtgtccgtggcactagtgggagaaggcgtgaaaataccgtttagttacataggccttggattcaacccatctacagatggctacctgtacgtcaacgtctcgtcacgagctccttgggttcaacagactccagacctatccgcgaacagcggctggggtattaaacaggttctagaaaaagagttactggccatccagatagggtgcgacaaccaaaaatttcccgaagaacccacaactaccccctcacttgtcacgacaacgctttccccaacaacgactttaaatccgaataacgaaaacacagacactacgccgacgcccaccggcgccagtgtagacggaaagcgcaatccagatgacattgacttctcgctgatcgtggacccccgatgcgtgacctctgtaaacctgcacttcgagctcaaggacgcgtgcatggactacaaaaaagagtcgccgttgtcgctgaaggggaaatatggagacagtgaactagtaaaacaggagattaaagacgtgggaaagaatcacaatatgtgcagtcttaacctcagccctggccattgagctgtttttattcggcaatataataaggtgattattgaacattaaacaaaacttatcccacaacgccgcaaca

SEQ ID NO: 49 - DNA, 1138 bp, Artificial Sequence
  Orf virus strain GO KP010354.1
ttgctgcgctatgtcgtgcccgactctgtgcgacaaagacagaagctcaccaaactcttcgtctctgttctccaagcaagaacactggagtgatttgccatttccgtctccaacatataattagcatccttgtttttatcttgtctttttatcagtttttatgttagttaaaacataaatagtaaagctaaaaagagtactctggaatcttgcaacagtcaggatgaaggtggtgttgttgctagtgctactgggggcgttaaccaacgcagcgcccgtcggcaaccagcgtattgacagtgaggagaaagcaaatttctgctcgacgcatcaaaatgaagtgtacgccaggttccggcttcagatgcgcgtgggtgtacggcacagtccgctctacactcccagcaacatgtgcatgatgaacatagaagactccatgatggataatgaatacacgtctacggccacaggtgatgcggacggagtgaacgtgtctgtggcactaatgggaaaaggcgtgagcataccgttaagttacataggcctcggattcaacccattgcttgcagatggctacctgtacgtcaacgtctcgtcacgagctccttgggttcaacaaactccggacctatccgcgaacggcggctggggtattaaacaggttctagaagaagagatactggccatccagatagggtgcgacaaccaaaaatttctagaagaacccacaacaacccaacccccctcacttgtcacgacaacgctttcctcaacaacgctaggtctgactgacgagaacacagacactacgccgacgaccaccagcgccagtgtagacagaaagcgcaatctagatgacattgacttctcgctgctcgtagacacccgatgcgtgacctcggtaaacctgcacttcgagattaaggacgcgtgtatggactacaaaaaagagtcgccgttgatgatgaaggggggatatggagacggcgaactagtaagaaaagagatcaaatacgtgggaacgaatcacactatgtgcagtctcaacctcagccctggctattgatttgtttttattcggcaatataataggtgattattgaacattaaacaaaacttatcccacaacgcctcaaca

SEQ ID NO: 50 - DNA, 1138 bp, Artificial Sequence
  Orf virus strain SJ1 KP010356.1
ttgctgcgctatgtcgtgcccgactctgtgcgacaaagacagaagctcaccaaactcttcgtctctgttctccaagtaagaacactggagtgatttgccatttccgtctccaacatataattagcatccttgtttttatcttgtctttttatcagtttttatgctagttaaaacataaatagtaaagctaaaaagagtactctggaatcttgcaacagtcaggatgaaggtggtggtgttgctggcgctactgggagcgttaaccaacgcagcgcccgttggcaaccagcgtcttaacagtaaagagaaagaagatttctgcttgacgcatccggatgaagtgtatgccaggttccggcttcatatgcgcgtgggtgtacgacacagtccgctttacactcccagcaacatgtgcatgatgaacatagaagactccatgatggataatgaatacacgtctacggccacgggtgatgcggacggagtgaacgtgtctgtggcactaatgggaaaaggcgtgagcataccgttaagttacataggcctaggattcaacccattgcttgcagatggctacttgtacgtcaacgtctcgtcacgagctccttgggttcaacagactccggacctatccgcgaacggcggctggggtattaaacaggttctagaagaagagatactggccatccagatagggtgcgacaaccaaaaatttctagaagaacccacaactccccaacccccctcacttgtcacgacaacgctttcctcaacaacgctaggtctgactgacgagaacacagacactacgccgacgaccaccagcgccagtgtagacagaaagcgcaatctagatgacattgacttctcgctgctcgtagacacccgatgcgtgacctcggtaaacctgcacttcgagatcaaggacgcgtgtatggactacaaaaaagagtcgccgttgatgatgaaggggggatatggagacggcgaactagtaagaaaagagatcaaatacgtgggaacgaatcacactatgtgcagtctcaacctcagccctggctattgatttgtttttattcggcaatataataggtgattattgaacattaaacaaaacttatcccacaacgccgcaaca

SEQ ID NO: 51 - DNA, 1040 bp, Artificial Sequence
  Orf virus strain SY17 MG712417.1
tcagcatccttgtttttatcctgtctttttatcagttttttagctagttaaaacataaatagtaaagctaaaaagaggagttctggagtcttgtaacaaccaggatgaaggcggtgttgttgctggcgttactgggagcgttcactaacgcagcgcccgtcggcaaccagcgtctcgatagtggggagaaagaaaaattctgctcgacgcatcaggatgaagtgtacgctaggttcaggcttcagatgcgcgtgggtgtacgacacagtcctttctacactcccagcaacatgtgcatgatggacatagaagactccgttgatgacatagaagaagactccattatagtaaaagaatttacgtctacggccacgggtgaggcggacggagtgaacgtgtccgtggcactagtgggagaagacgtgaaaataccgttaagttacataggccttggattcaacccatctacagatggctacctgtacgtcaacgtctcgtcacgagctccttgggttcaacagactctagacctatctgcgaacgacagctggggtattaaacaggttctagaaaaagagttactggccatccaaatagggtgcgacaaccaaaaatttcctgaagaacccacaaccacccaacctccctcacttgtcacgacaacgctttcctcaacaactccagatctgaatgacgaaaacacagacactacgccgacgaccactggcgccagtgtagacagaaagcgcaatccagctgactttagtttctcgctgctcgtggacccccgatgcgtgacttctgtagacctgcacgtcgagctcagggacgcgtgcatggagtacaaagaaacgtcgccgttgtcgctgaagggggaatatggagacggcgaactagtaaaaaaggagatcaaagacgtgggaaagaatcacaatatgtgcagtcttaacctcaaccctggcaattaagctgtttttattcggcaatataataggtgattattgaacattaaacaaaacttatcccacaacgccgcaaca

SEQ ID NO: 52 - DNA, 1035 bp, Artificial Sequence
  Orf virus strain OV-HN3/12 KY053526.1
tcaacatccttgtttttatcctgtctttttatcagtttttagctagttaaaacataaatagtaaagctaaaaagaggagttctggagtcttgcaataaccaggatgaaggcggtgttgttgctggcgttactgggagcgttcaccaacgcagcgcctttgttagaaagccagcgttctaacagtgaggagaaagcaaatttctgctcgacgcataataatgaagtgtacgccaggttcaggcttcagatgcgcgtgggtgtacgacacagtcctttctacactcccagcaacatgtgcatgatggacatagaagactccgttgaggacatagaagagtccacagagaaagaatacgcgtctacggccacgggtgaggcggccggagtgaacgtgtcagtggcactagtgggagaaggcgtgagcataccgtttagttacataggccttggattcaacccgtcgcttgaagatagctacctgtacgttaacgtctcgtcacgagctccttgggttaaacagacttcggacctatccgcgaacggcggctggggtattaaacaggttctagaaaaagagttactggccatccaaatagggtgcgacaaccaaaaatttcccgaagaacccacaactacacccccctcacctgtcacgacaacgctttcctcaacaactccagatctgaatgaagaaaacacagacactacgccgacgaccaccagcgccagtgtagacagaaggcgcaatctagatgacattgacttctcgctgctcgtggacccccgatgcgtgacctctgtagacctacacgtcgagctcagggacgcgtgcatggactacaaacaagagtcgccgttgtcgctgaaggggaaatatggagacggcgagctagtaaaaaaggagatcaaagacgtgggaaagaattacaatatgtgcagtcttaacctcaaccctggcaattgagatgtttttattcggcaatataatagggattattgaacattaaacaaaacttatcccacaacgccgcaaca

SEQ ID NO: 53 - DNA, 1163 bp, Artificial Sequence
  Orf virus strain NP KP010355.1
ttgctgcgctatgtcgtacctgactctgtgtacaaagacagcagctcaccagactcttcgtccctgttctctaagcaagaacactggagtgagttgccatttccgtctccgacatataattagcatccttgtttttatcctgtctttttatcagtttttatgctagttaaaacataaatagtaaagctaaaaagagtactctggaatcttgcaacaatcaggatgaaggtggtggtgttgctggcgctactgggagcgttaaccaacgcagcgcccgtcggcaaccagcgtcttaacagtaaagagaaagaagatttctgctcgacgcatccggatgaagtgtacgccaggttccggcttcagatgcgcgtgggtgtacgacacagtccgctctacgttcccagcaacatgtgcatgatggacatagaagactctgttgatgacatagaagaagactccattatagtaaaagaattcacgtctacagccacgggtgaggcggccggagtgaacgtgtccgtggcactagtgggagaaggcgtgagcataccgtttagttacataggccttggattcaacccgtcgcttgaagatagctacttgtacgtcaacgtctcgtcacgagctccttgggttcaacagactccagacttatctgcaaacgacagctggggtattaaacaggttctagaaaaagagttactggccatccaaatagggtgcgacaaccaaaaatttcccgaagaacccacaaccacccaacccccctcacttgtcacgacaacgctttcctcaacaactctagatctgaatgacgaaaacacagacactacgccgccaaccaccaccagcgccagcgtaaacaaaaagcgcaatccagatgactttgacttctcgctgctcgtggacccccgatgcgtgacctctgtagacctgcacgtcgagctcagggacgcgtgcatagactacaaagaaacgtcgcagttgtcgctgaagggggaatatggagacggcgaactaataaaaaaggagatcaaagacgtgggaaaggatcacaatatgtgcagtcttaacctcaaccctggcaattgagctgtttttattcggcgtataataggtgattattgaacattaaacaaaacttatcccacaacgccgcaaca

SEQ ID NO: 54 - DNA, 1183 bp, Artificial Sequence
  Orf virus strain B029 KF837136.1
ttgctgctatgtcgtacccgactctgtgctacaaagacagcagctcaccagactcttcgtccctgttctccaagcaagaacactggagtgagttgccatttccgtctccaaccatataattagcatccttgtttttatcctgtatttttatcagtttttatgctagttaaaacataaatagtaaggctaaaaagaagagttctagaatcttgcaacaaccaagatgaaggcggtgttgttgctagcgctactgggagcgttcaccaacgcagcgcctttgttaagcaaccagcgtcttggcagtgcagaggaagaaaaattctgctcgacgcatcatgacaaagtgtacgccaggttctggcttcagatgcgtgtgggtgtacgacacagttcgctctacgctcccagcaacatgtgcatgatggacatagaagactctacggtggacatagaaggctccacggaaacagaagactccacggtggaaaaagaatacacgtctgcggcaacgggtgatgcgaacggagtgaacgtgtccgtggcactaatgggagaaggcgtgagcataccgctaagttacataggcctcggattcaacccattgcttaaagatggctacctgtacgtcaacgtctcgtcacgggctccttgggatcaacagactctggacctatccgcgaacgacggctggggtattaaacaggttctagaaaaagagatactggccatccagatagggtgcgacaaccaaaaatttcccgaagaacccacaactacccaacccccctcacctgtcacaacaacgctttccccaacaacgactttaaacccgaataacgaaaacacagacactacgccgacgcccaccggcgccagtgtagacggaaagcgcaatccagatgacattgacttctcgctgatcgtggacccccgatgcgtgacctctgtaaacctgcacttcgagatcaaggacgcgtgcatggactacaaacaagagtcgccgttgtcgctgaaggggaaatatggagacggtgaactagtaaaagaggagattaaagacgtgggaaagaaccacaatatgtgcagtcttaacctcagccctggccattgagctgtttttattcggcaatataataggtgattattgaacattaaacaaaacttatcccacaacgccgcaaca

SEQ ID NO: 55 - DNA, 21 bp, Artificial Sequence
  Forward primer
cgtgatttgggaatatgcacc

SEQ ID NO: 56 - DNA, 20 bp, Artificial Sequence
  Reverse primer
gtatctatcgtcgggaccag

SEQ ID NO: 57 - DNA, 22 bp, Artificial Sequence
  5′truncated ORFV112 gene
cttatcccacaacgccgcaaca

SEQ ID NO: 58 - protein, 179 aa, Artificial Sequence
  Full peptide sequence of the hypothetical protein 111
MSRLQILTSFGQIYAPDEARLREIARDLGICTIKRAFGDMLYGFIDFNPVPLTQVNMLMPNCYFAVNGNLLPCTEDFRLRLPATEISAAYLTKTGRTILCGRDFNIVAPSGFKPSMRLRNLSHVSALVEILELYDESGEYQFVLGPSAQLMLRLMEKENVCLFGRGWCIVDLRKLDVAI

SEQ ID NO: 59 - DNA, 794 bp, Artificial Sequence
  Full DNA sequence of the ORFV111 gene
aaatatactgtttgaacgcaaagacgccatgtcgcgacttcaaatactgacctcatttggacaaatctacgcacctgacgaagctcggctgcgagagatcgcgcgtgatttgggaatatgcaccataaaacgcgcattcggcgacatgctgtacggctttatagacttcaacccggtgcccctgacccaagtaaacatgctcatgcccaactgctacttcgcggtcaacggcaacctgcttccgtgcacggaggacttccggctcagactcccggcaacggagatctctgcggcctacctgacgaaaacgggacggacgatcctgtgcggtagagacttcaacatagtagcgccgtcggggttcaagccgtccatgcggctgcgcaacctcagtcacgtgtctgcgcttgtagagattttagagctttacgacgagtccggagagtaccaattcgtgctcggccccagcgcgcagttaatgctgcggctaatggagaaggagaatgtctgtctgtttggcagaggttggtgcatagtggacctgcgcaagctggacgtagccatataattgctgctgctatgtcgtgcccgactctgtgcgacaaagaagcagcttaccagacgactcttcgttcatgttctctaagcaagaacactggagtgagttgccatttccgtctccaaccatataattagcatccttgtttttatcctgtatttttatcagtttttagctagttaaaacataaatagtaaagctaaaaagaggagttctggaatcttgcaacgacagg

SEQ ID NO: 60 - DNA, 485 bp, Artificial Sequence
  3′ truncated type of the ORFV111 gene
aaatatactgtttgaacgcaaagacgccatgtcgcgacttcaaatactgacctcatttggacaaatctacgcacctgacgaagctcggctgcgagagatcgcgcgtgatttgggaatatgcaccataaaacgcgcattcggcgacatgctgtacggctttatagacttcaacccggtgcccctgacccaagtaaacatgctcatgcccaactgctacttcgcggtcaacggcaacctgcttccgtgcacggaggacttccggctcagactcccggcaacggagatctctgcggcctacctgacgaaaacgggacggacgatcctgtgcggtagagacttcaacatagtagcgccgtcggggttcaagccgtccatgcggctgcgcaacctcagtcacgtgtctgcgcttgtagagattttagagctttacgacgagtccggagagtaccaattcgtgctcggccccagcgcgcagttaatgctgc

SEQ ID NO: 61 - protein, 179 aa, Artificial Sequence
  Orf virus strain YX AKU76601.1
MSQLQILTSFGQIYAPDEARLREIARDLGICTIKRAFGDMLYGFIDFNPVPLTQVNMLMSDFYFAVNGNLLPCTEDFRLRLPATDISAAYLTKTGRTILCGKDFNIVAPSGFKPSMRLRNLSHVSALVEILEFYSESGEYQFVLGPSAQFMLRLMEKENVCLFGSGWCIVDLRKLDVTI

SEQ ID NO: 62 - protein, 179 aa, Artificial Sequence
  Orf virus strain NA1/11 AHZ33809.1
MSRLQILTSFGQIYAPDEARLREIARDLGVCTIKRAFGDMLYGFIDFDPVPLTQVNMLMPNCYFAVNGNLLPCTEDFRLRLPATEISAAYLTRTGRTILCGKDFNIVAPSGFKPSMRLRDLSHVSALVEILEIYNESGEYQFVLGPSAQFMLRLMEKENVCLFGSGWCIVDLRKLDVPI

SEQ ID NO: 63 - protein, 179 aa, Artificial Sequence
  Orf virus strain NZ2 ABA00629.1
MSRLQILTSFGQIFAPDEARLREIARDLGICTIKRAFGDMLYGFIDFDPVPLTQVNMLMSNCYFAVNGNLLPCTEDFRLRLPATEISAAYLTRTGRTILCGKDFNIVAPSGFKPSMRLRDLSHVSALVEILEVYDESGEYQFVLGPSAQFMLRLMEKENVCLFGSGWCIVDLRKLDVPI

SEQ ID NO: 64 - protein, 179 aa, Artificial Sequence
  Orf virus strain NA17 AYM26052.1
MSRLQILTSFGQIYAPDEARLREIARDLGICTIKRAFGDMLYGFIDFNPVPLTQVNMLMSDCYFAVNGNLLPCTEDFRLRLPATEISAAYLTKTGRTILCGKDFNIIAPSGFKPSMRLRNLSHVSALVEILEFYSESGEYQFVLGPSAQFMLRLMEKENVCLFGSGWCIVDLRKLDVTI

SEQ ID NO: 65 - protein, 179 aa, Artificial Sequence
  Orf virus strain OV-SA00 AAR98336.1
MSRLQILTSFGQIYAPDEARLREIARDLGICTIKRAFGDMLYGFIDFNPVPLTQVNMLMSNCYFAVNGNLLPCTEDFRLRLPATEISAAYLTKTGRTILCGKDFNIIAPLGFKPSMRLRNLSHVSALVEILEFYSESGEYQFVLGPSAQFMLRLMEKENVCLFGSGWCIVDLRKLDVTI

SEQ ID NO: 66 - protein, 198 aa, Artificial Sequence
  Orf virus strain OV-IA82 AAR98206.1
MHEGDARITENILFERKDAMSRLQILTSFGQIYAPDEARLREIARDLGICTIKRAFGDMLYGFIDFNPVPLTQVNMLMSNCYFAVNGNLLPCTEDFRLRLPATEISAAYLTRTGRTILCGKDFNIVAPSGFKPSMRLRDLSHVSALVEILELYDESGDYQFVLGPSAQFMLRLMEKENVCLFGNGWCIVDLRKLDVTI

SEQ ID NO: 67 - protein, 179 aa, Artificial Sequence; identical to SEQ ID NO: 64
  Orf virus strain GO AKU76733.1

SEQ ID NO: 68 - protein, 179 aa, Artificial Sequence
  Orf virus strain SJ1 AKU76989.1
MSQLQILTSFGQIYAPDEARLREIARDLGICTIKRAFGDMLYGFIDFNPVPLTQVNMLMSNCYFAVNGNLLPCTEDFRLRLPATEISAAYLTKTGRTILCGKDFNIVAPSGFKPSMRLRNLSHVSALVEILEFYSESGEYQFVLGPSAQFMLRLMEKENVCLFGSGWCIVDLRKLDVTI

SEQ ID NO: 69 - protein, 179 aa, Artificial Sequence
  Orf virus strain SY17 AYN61059.1
MSRLQILTSFGQIYAPDEARLREIARDLGICTIKRAFGDMLYGFIDFNPVPLTQVNMLMPNCYFAVNGNLLPCTEDFRLRLPATEISAAYLTRTGRTILCGKDFNIVAPSGFKPSMRLRDLSHVSALVEILEIYNESGEYQFVLGPSAQFMLRLMEKENVCLFGSGWCIVDLRKLDVTI

SEQ ID NO: 70 - protein, 179 aa, Artificial Sequence; identical to SEQ ID NO: 62
  Orf virus strain OV-HN3/12 ASY92408.1

SEQ ID NO: 71 - protein, 179 aa, Artificial Sequence
  Orf virus strain NP AKU76865.1
MSRLQILTSFGQIYAPDEARLREIARDLGICTIKRAFGDMLYGFIDFNPVPLTQVNMLMSDFYFAVNGNLLPCTEDFRLRLPATDISAAYLTKTGRTILCGKDFNIVAPSGFKPSMRLRNLSHVSALVEILEFYSESGEYQFVLGPSAQFMLRLMEKENVCLFGSGWCIVDLRKLDVTI

SEQ ID NO: 72 - protein, 179 aa, Artificial Sequence
  Orf virus strain B029 AIO03573.1
MSRLQILTSFGQIYAPDEARLREIARDLGICTIKRAFGDMLYGFIDFNPVPLTQVNMLMSNCYFAVNGNLLPCTEDFRLRLPATEISAAYLTKTGRTILCGKDFNIVAPSGFKPSMRLRDLSHVSALVEILELYDESGEYQFVLGPSAQFMLRLMEKENVCLFGNGWCIVDLRKLDVTI

SEQ ID NO: 73 - DNA, 795 bp, Artificial Sequence
  Orf virus strain YX KP010353.1
ctgaaaatatactgtttgaatgcaaagacgccatgtcgcaacttcaaatactgacctcatttggacaaatctacgcacccgacgaagctcggctgcgcgagatcgcgcgtgatttgggaatatgcaccataaaacgcgcattcggcgacatgctgtacggctttatagacttcaacccggtgccccttacccaagtaaacatgctcatgtccgacttctacttcgcggtcaacggcaacctgcttccgtgcacggaggacttccggctcagactcccggcgacggatatctctgcggcctacctgacgaaaacgggacggacgatcctgtgcggcaaagacttcaacatagtggcgccgtcagggttcaagccgtccatgcggctgcgcaacctcagtcacgtgtctgcgcttgtagagatcttggagttttacagcgagtccggggagtaccaattcgtgctcggccccagcgcgcagttcatgctgcggctgatggagaaggagaacgtctgtctgttcggcagcggatggtgcatagtggacctgcgcaagctggacgtaaccatataattgctgctgctatatcgtacctgactctgtgttttaaagacagcagctcaccgactcttcgtccctgttctccaagcaagaacactggagtgatttgccatttccgtctccaacatataattagcatccttgtttttatcttgtctttttatcagtttttatgttagttaaaacataaatagtaaagctaaaaagagtactctggaatcttgcaacagtcagg

SEQ ID NO: 74 - DNA, 675 bp, Artificial Sequence
  Orf virus strain NA1/11 KF234407.1
ctgaaaatatactgtttgaacgcaaagacgccatgtcgcgacttcaaatactgacctcatttggacaaatctacgcacccgacgaagctcggctgcgcgagatcgcgcgtgatttgggagtatgcaccataaaacgcgcattcggcgacatgctgtacggctttatagacttcgacccggttcccctgacccaagtaaacatgctcatgcccaactgctacttcgcggttaacggcaacctgcttccgtgcacggaggacttcaggctcagactcccggcaacggagatctccgcggcctacctgacgagaacgggacggacgatcctgtgcggcaaagacttcaacatagtggcgccgtcagggttcaagccgtccatgcggctgcgcgaccttagtcacgtgtctgcgcttgtagagatcctggaaatctacaacgagtccggggagtaccaattcgtgctcggccccagcgcgcagttcatgctgcggctgatggagaaggaaaacgtctgtctgttcggcagcgggtggtgcatagtggacctgcgcaagctggacgtacccatatatcaacatccttgtttttatcctgtctttttatcagttttttagctagttaaaacataaatagtaaagctaaaaagaggagttctggagtcttgcaataaccagg

SEQ ID NO: 75 - protein, 676 aa, Artificial Sequence
  Orf virus strain NZ2 DQ184476.1
CTGAAAATATACTGTTTGAACGCAAAGACGCCATGTCGCGACTTCAAATACTGACCTCATTTGGACAAATCTTCGCACCCGACGAAGCTCGGCTGCGCGAGATCGCGCGTGATTTGGGAATATGCACCATAAAACGCGCATTCGGCGACATGCTGTACGGCTTTATAGACTTCGACCCGGTGCCCCTGACCCAAGTAAACATGCTCATGTCCAACTGCTACTTCGCGGTCAACGGCAACCTGCTTCCGTGCACGGAGGACTTCCGGCTCAGACTCCCGGCAACGGAGATCTCTGCGGCCTACCTGACGAGAACGGGACGGACGATCCTGTGCGGCAAAGACTTCAACATAGTAGCGCCGTCGGGGTTCAAGCCGTCCATGCGGCTGCGCGACCTCAGTCACGTGTCTGCGCTTGTAGAGATCCTGGAAGTCTACGACGAGTCCGGGGAGTACCAATTCGTGCTCGGCCCCAGCGCGCAGTTCATGCTGCGGCTGATGGAGAAGGAGAACGTCTGTCTGTTCGGCAGCGGGTGGTGCATAGTGGACCTGCGCAAGCTGGACGTACCCATATAATCAGCATCCTTGTTTTTATCCTGTCTTTTTATCAGTTTTTTAGCTAGTTAAAACATAAATAGTAAAGCTAAAAAGAGGAGTTCTGGAGTCTTGCAACAACCAGG

SEQ ID NO: 76 - protein, 796 aa, Artificial Sequence
  Orf virus strain NA17 MG674916.2
CTGAAAATATACTGTTTGAACGCAAAGACGCCATGTCGCGACTTCAAATACTGACCTCATTTGGACAAATCTACGCACCCGACGAAGCTCGGCTGCGCGAGATCGCGCGTGACTTGGGAATATGCACCATAAAACGCGCATTCGGCGACATGCTGTACGGCTTTATAGACTTCAACCCGGTGCCCCTTACCCAAGTAAACATGCTCATGTCCGACTGCTACTTCGCGGTCAACGGCAACCTGCTTCCGTGCACGGAGGACTTCCGGCTCAGACTCCCGGCGACGGAGATCTCCGCGGCCTACCTGACGAAAACGGGACGGACGATCCTGTGCGGCAAAGACTTCAACATAATAGCGCCGTCGGGTTTCAAGCCGTCCATGCGGCTGCGCAACCTCAGTCACGTGTCTGCGCTTGTAGAGATCTTGGAGTTTTACAGCGAGTCCGGGGAGTACCAATTCGTGCTCGGCCCCAGCGCGCAGTTCATGCTGCGGCTGATGGAGAAGGAAAACGTCTGTCTGTTTGGCAGCGGTTGGTGCATAGTGGACCTGCGCAAGCTGGACGTAACCATATAATTGCTGCTGCTATATCGTACCTGACTCTGTGTTACAAAGACAGCAGCTCACCAGACTCTTCGTCCCTGTTCTCTAAGCAAGAACACTGGAGTGAGTTGCCATTTCCGTCTCCAACATATAATTAGCATCCTTGTTTTTATCTTGTCTTTTTATCAGTTTTTATGCTAGTTAAAACATAAATAGTAAAGCTAAAAAGAGTACTCTGGAATCTTGCAACAATCAGG

SEQ ID NO: 77 - DNA, 791 bp, Artificial Sequence
  Orf virus strain OV-SA00 AY386264.1
aaatatactgtttgaacgcaaagacgccatgtcgcgacttcaaatactgacctcatttggacaaatctacgcacccgacgaagctcggctgcgcgagatcgcgcgtgacttgggaatatgcaccataaaacgcgcattcggcgacatgctgtacggctttatagacttcaacccggtgcccctgacccaagtaaacatgctcatgtccaactgttacttcgcggtcaacggcaacctgcttccgtgcacggaggacttccggctcagactcccggcgacggagatctctgcggcctacctgacgaaaacgggacggacgatcctgtgcggcaaagacttcaacataatagcgccgttggggttcaagccgtccatgcggctgcgcaacctcagtcacgtgtctgcgcttgtagagatcttggagttttacagcgagtccggggagtaccaatttgtgctcggccccagcgcgcagttcatgctgcgtctgatggagaaggagaacgtctgtctgttcggcagcggttggtgcatagtggacctgcgcaagctagacgtaactatataattgctgcgctatgtcgtgcccgactctgtgcgacaaagacagaagctcaccaaactcttcgtctctgttctccaagcaagaacactggagtgatttgccatttccgtctccaacatataattagcatccttgtttttatcttgtctttttatcagtttttatgttagttaaaacataaatagtaaagctaaaaagagtactctggaatcttgcaacagtcagg

SEQ ID NO: 78 - DNA, 822 bp, Artificial Sequence
  Orf virus strain OV-IA82 AY386263.1
atgcatgaaggagatgcacggataactgaaaatatactgtttgaacgcaaagacgccatgtcgcgacttcaaatactgacctcatttggacaaatctacgcacccgacgaagctcggctgcgcgagatcgcgcgtgatttgggaatatgcaccataaaacgcgcattcggcgacatgctgtacggctttatagacttcaacccggtgcccctgacccaagtaaacatgctcatgtccaactgctacttcgcggtcaacggcaacctgcttccgtgcacggaggacttccggctcagactcccggcaacggagatctctgcggcctacctgacgagaacgggacggacgatcctgtgcggcaaagacttcaacatagtggcgccgtcagggttcaagccgtccatgcggctgcgcgaccttagtcacgtgtctgcgcttgtagagatcctggagctctacgacgagtccggggattaccaattcgtgctcggccccagcgcgcagttcatgctgcggctgatggagaaggagaatgtctgtctgttcggcaacggttggtgcatagtggacctgcgcaagctagacgtaaccatataattgctgctgctatgtcgtgcccgactctgtgcgacaaagacagcggctaaccagactcttcgtccctgttctccaagcaaaaaactggagtgagttgccatttccgtctccaaccatataattagcatccttgtttttatcctgtatttttatcagtttttatgctagttaaaacataaatagtaaggctaaaaagaagagttctagaatcttgcaacaaccaag

SEQ ID NO: 79 - DNA, 791 bp, Artificial Sequence
  Orf virus strain GO KP010354.1
aaatatactgtttgaacgcaaagacgccatgtcgcgacttcaaatactgacctcatttggacaaatctacgcacccgacgaagctcggctgcgcgagatcgcgcgtgacttgggaatatgcaccataaaacgcgcattcggcgacatgctgtacggctttatagacttcaacccggtgccccttacccaagtaaacatgctcatgtctgactgctacttcgcggtcaacggcaacctgcttccgtgcacggaggacttccggctcagactcccggcgacggagatctccgcggcctacctgacgaaaacgggacggacgatcctgtgcggcaaagacttcaacataatagcgccgtcgggtttcaagccgtccatgcggctgcgcaacctcagtcacgtgtctgcgcttgtagaaatcttggagttttacagcgagtccggggagtaccaattcgtgctcggccccagcgcgcagttcatgctgcggctgatggagaaggagaatgtctgtctgttcggcagcggttggtgcatagtggacctgcgcaagctagacgtaaccatataattgctgcgctatgtcgtgcccgactctgtgcgacaaagacagaagctcaccaaactcttcgtctctgttctccaagcaagaacactggagtgatttgccatttccgtctccaacatataattagcatccttgtttttatcttgtctttttatcagtttttatgttagttaaaacataaatagtaaagctaaaaagagtactctggaatcttgcaacagtcagg

SEQ ID NO: 80 - DNA, 795 bp, Artificial Sequence
  Orf virus strain SJ1 KP010356.1
ctgaaaatatactgtttgaatgcaaagacgccatgtcgcaacttcaaatactgacctcatttggacaaatctacgcacccgacgaagctcggctgcgcgagatcgcgcgtgatttgggaatatgcaccataaaacgcgcattcggcgacatgctgtacggctttatagacttcaacccggtgccccttacccaagtaaacatgctcatgtccaactgctacttcgcagtcaacggcaacctgcttccgtgcacggaggacttccggctcagactcccggcgacggagatctctgcggcctacctgacgaaaacgggacggacgatcctgtgcggcaaagacttcaacatagtggcgccgtcggggttcaagccgtccatgcggctgcgcaacctcagtcacgtgtctgcgcttgtagagatcttggagttttacagcgagtccggggagtaccaattcgtgctcggccccagcgcgcagttcatgctgcggctgatggagaaggagaatgtctgtctgttcggcagcggttggtgcatagtggacctgcgcaagctagacgtaaccatataattgctgcgctatgtcgtgcccgactctgtgcgacaaagacagaagctcaccaaactcttcgtctctgttctccaagtaagaacactggagtgatttgccatttccgtctccaacatataattagcatccttgtttttatcttgtctttttatcagtttttatgctagttaaaacataaatagtaaagctaaaaagagtactctggaatcttgcaacagtcagg

SEQ ID NO: 81 - DNA, 676 bp, Artificial Sequence
  Orf virus strain SY17 MG712417.1
ctgaaaatatactgtttgaacgcaaagacgccatgtcgcgacttcaaatactgacctcatttggacaaatctacgcacccgacgaagctcggctgcgcgagatcgcgcgtgatttgggaatatgcaccataaaacgcgcattcggcgacatgctgtacggctttatagacttcaacccggtgcccctgacccaagtaaacatgctcatgcccaactgctacttcgctgtcaacggcaacctgcttccgtgcacggaggacttccggcttagactcccggcaacggagatctctgcggcctacctaacgagaacgggacggacgatcctgtgcggcaaagacttcaacatagtggcgccgtcagggttcaagccgtccatgcggctgcgcgacctcagtcacgtgtctgcgcttgtagagatcctggaaatctacaacgagtccggggagtaccaattcgtgctcggccccagcgcgcagttcatgctgcggctgatggagaaggaaaacgtctgtctgttcggcagcgggtggtgcatagtggacctgcgcaagctggacgtaaccatataatcagcatccttgtttttatcctgtctttttatcagttttttagctagttaaaacataaatagtaaagctaaaaagaggagttctggagtcttgtaacaaccagg

SEQ ID NO: 82 - DNA, 675 bp, Artificial Sequence
  Orf virus strain OV-HN3/12 KY053526.1
ctgaaaatatactgtttgaacgcaaagacgccatgtcgcgacttcaaatactgacctcatttggacaaatctacgcacccgacgaagctcggctgcgcgagatcgcgcgtgatttgggagtatgcaccataaaacgcgcattcggcgacatgctgtacggctttatagacttcgacccggttcccctgacccaagtaaacatgctcatgcccaactgctacttcgcggttaacggcaacctgcttccgtgcacggaggacttcaggctcagactcccggcaacggagatctccgcggcctacctgacgagaacgggacggacgatcctgtgcggcaaagacttcaacatagtggcgccgtcagggttcaagccgtccatgcggctgcgcgaccttagtcacgtgtctgcgcttgtagagatcctggaaatctacaacgagtccggggagtaccaattcgtgctcggccccagcgcgcagttcatgctgcggctgatggagaaggaaaacgtctgtctgttcggcagcgggtggtgcatagtggacctgcgcaagctggacgtacccatataatcaacatccttgtttttatcctgtctttttatcagtttttagctagttaaaacataaatagtaaagctaaaaagaggagttctggagtcttgcaataaccagg

SEQ ID NO: 83 - DNA, 794 bp, Artificial Sequence
  Orf virus strain NP KP010355.1
ctgaaaatatactgtttgaacgcaaagacgccatgtcgcgacttcaaatactgacctcatttggacaaatctacgcacccgacgaagctcggctgcgcgagatcgcgcgtgacttgggaatatgcaccataaaacgcgcattcggcgacatgctgtacggctttatagacttcaacccggtgccccttacccaagtaaacatgctcatgtccgacttctacttcgcggtcaacggcaacctgcttccgtgcacggaggacttccggctcagactcccggcgacggatatctctgcggcctacctgacgaaaacgggacggacgatcctgtgcggcaaagacttcaacatagtggcgccgtcagggttcaagccgtccatgcggctgcgcaacctcagtcacgtgtctgcgcttgtagagatcttggagttttacagcgagtccggggagtaccaatttgtgctcggccccagcgcgcagttcatgctgcggctgatggagaaggagaacgtctgtctgttcggcagcggttggtgcatagtggacctgcgcaagctagacgtaaccatataattgctgcgctatgtcgtacctgactctgtgtacaaagacagcagctcaccagactcttcgtccctgttctctaagcaagaacactggagtgagttgccatttccgtctccgacatataattagcatccttgtttttatcctgtctttttatcagtttttatgctagttaaaacataaatagtaaagctaaaaagagtactctggaatcttgcaacaatcagg

SEQ ID NO: 84 - DNA, 790 bp, Artificial Sequence
  Orf virus strain B029 KF837136.1
aaatatactgtttgaacgcaaagacaccatgtcgcgacttcaaatactgacctcatttggacaaatctacgcacccacgaagctcggctgcgcgagatcgcgcgtgatttgggaatatgcaccataaaacgcgcattcggcgacatgctgtacggctttatagacttcaacccggtgcccctgacccaagtaaacatgctcatgtccaactgctacttcgcggtcaacggcaacctgcttccgtgcacggaggacttccggctcagactcccggcaacggagatctctgcagcctacctgacgaaaacgggacggacgatcctgtgcggcaaagacttcaacatagtggcgccgtcggggttcaagccgtccatgcggctgcgcgacctcagtcacgtgtctgcgcttgtagagatcctggagctctacgacgagtccggggagtaccaattcgtgctcggccccagcgcgcagttcatgctgcggctgatggagaaggagaatgtctgtctgttcggcaacgggtggtgcatagtggacctgcgcaagctagacgtaaccatataattgctgctatgtcgtacccgactctgtgctacaaagacagcagctcaccagactcttcgtccctgttctccaagcaagaacactggagtgagttgccatttccgtctccaaccatataattagcatccttgtttttatcctgtatttttatcagtttttatgctagttaaaacataaatagtaaggctaaaaagaagagttctagaatcttgcaacaaccaag

SEQ ID NO: 85 - protein, 154 aa, Artificial Sequence
  Amino acid sequence of the ORFV111 gene of the POV-601-1A1 strain (mutant)
MSRLQILTSFGQIYAPDEARLREIARDLGICTIKRAFGDMLYGFIDFNPVPLTQVNMLMPNCYFAVNGNLLPCTEDFRLRLPATEISAAYLTKTGRTILCGRDFNIVAPSGFKPSMRLRNLSHVSALVEILELYDESGEYQFVLGPSAQLMLRR

SEQ ID NO: 86 - protein, 183 aa, Artificial Sequence
  Amino acid sequence of the ORFV112 gene of the POV-601-1A1 strain (mutant)
GVSIPLSYIGLRFNPSLTDGYLYVNVSSRAPWDQQTLDLSANDGWGIKQVLEKEILAIQIGCDNQKFPEEPTTTQPPSPVTTTLSSTTLDPNDENTDTTPTTTGDSVDGKRNPDDFDFSLIVDPRCVTSVNLHFEIKDACMDHKESSPLSLKGEYGDGELVRKEIKNVGKDHNMCSLNLSPGH

(19) United States
(12) Patent Application Publication
(10) Pub. No.: US 2023/0340424 A1
(43) Pub. Date: Oct. 26, 2023

(54) MUTANT ORF VIRUSES AND USES THEREOF

(71) Applicant: Suzhou Prajna Biotech Co., Ltd., Suzhou (CN)

(72) Inventor: Minjie HU, Suzhou (CN)

(21) Appl. No.: 18/041,234

(22) PCT Filed: Aug. 13, 2021
(86) PCT No.: PCT/CN2021/112408

§ 371 Date: Feb. 10, 2023

(30) Foreign Application Priority Data
Aug. 13, 2020 (CN) 202010813096.9

(51) Int. Cl. C12N 7/00; A61K 35/768; A61P 35/00

(52) U.S. Cl. CPC C12N 7/00; A61K 35/768; A61P 35/00; C12N 2710/24221; C12N 2710/24232; C12N 2710/24271

(57) ABSTRACT

Provided are the use of a mutant Ovis spp. infectious pustular dermatitis virus (ORFV) and a pharmaceutical composition thereof in the treatment of cancer.

### RELATED APPLICATIONS

This application claims priority to China Provisional Application Ser. No. 202010813096.9 filed Aug. 13, 2020.

### FIELD

The present invention relates to the biotechnology field, more specifically, relates to mutant Orf viruses and uses thereof as pharmaceutical composition in treating cancers.

### BACKGROUND OF THE INVENTION

The Orf virus (ORFV), is a member of the Parapoxvirus (PPV) genus in the Poxviridae family. The PPV genus includes other members, such as the Bovine papular stomatitis virus, Pseudocowpox virus, Parapoxvirus of red deer in New Zealand, Sealpox and other viruses. The ORFV can cause contagious, and epitheliotropic infections in sheep and goats. When animals are infected by an ORFV, erythema initially emerges on the lips, tongue, nose, breast et al, and subsequently progresses to papule, vesicle, pustule, and scab, which are often characterized by proliferative skin inflammation.

Patent CN 104017776 B discloses an attenuated ORFV, obtained by isolation and cell subculture, for vaccine development. Patent CN 108026542 A describes the use of the viral strain D1701 to make a recombinant ORFV vector for expressing viral and tumor antigens, etc., for the preparation of vaccines.

The oncolytic viral therapy is a type of the systemic therapy that employs naturally existing or engineered viruses to treat cancers. The special gene mutations that originally promote the proliferation and survival of tumor cells, but also promote the growth of the viruses having oncolytic functionality (oncolytic virus, OV) within the tumor cells, which is one of the reasons an OV can precisely attacks tumor cells^{[1, 2, 3]}. Following infection, an oncolytic virus promotes tumor cell lysis and death at a suitable time point within its viral life cycle, leading to a release of infectious viral virions, further infecting neighboring tumor cells. At the same time, neoantigens released during oncolysis activate the host immune system, and reattack the tumor the second time^{[4]}. Currently, more than 90 ongoing or completed clinical trials using oncolytic viruses for the treatment of human malignancies have been documented worldwide. Among them, TVEC ((talimogene laherparepvec) has been successfully approved by FDA^{[5]}.

ORFV is a double-stranded DNA virus with a genome of 134-139 kb^{[6]}. It is an ovoid in shape, resembling a ball of yarn, and measures approximately 230-280 mn (long)×150-200 nm (wide). ORFV has the following characteristics: 1) an ORFV, like other members of the poxviridae family, only replicates in the cytoplasm of a host cell and does not enter the nucleus, therefore (its DNA) will not integrates into the host cell's genome, so, its safety is high and its probability of carcinogenicity is extremely low.^{[7, 10]}; 2) an ORFV could infect a human with broken or scarified skin through contact of the infected area of an animal, producing clinically mild pustules at the site of infection (usually on fingers) with basically no other adverse effects and life-threatening conditions, and resolving spontaneously within 6-8 weeks^{[8, 11]}; 3) ORFV infection does not rely on specific cell surface receptors^{[9, 10]}, and therefore could provide a way to overcome the heterogenicity problem of a solid tumor; 4) an ORFV has a large genome, which is conducive to the insertion and efficient expression of foreign genes; 5) ORFV's viral genome is stable and the fidelity of replication is high; 6) little or no neutralizing antibodies against an ORFV are produced in the host, so multiple infections can occur^{[}11], and repeated intravenous injections of an ORFV drug into cancer patients can be achieved; 7) ORFV infection can induce both innate, and adaptive anti-tumor immune responses within a host^{[12]}; 8) Vaccinia, another member of the poxvirus family, has been used as a vaccine against smallpox infection in billions of people worldwide, setting the basis for low risks in future clinical applications of an ORFV drug; 9) an ORFV has the capability of turning a “Cold” tumor into a “Hot” one^{[18]}, which is ORFV's own properties. Based on these special characteristics discussed above, ORFV will likely become a new type of an oncolytic virus to treat solid tumors.

An ORFV viral strain usually has 130-134 (protein encoding) genes. The currently confirmed virulence factors include the orf virus interferon-resistance factor (OVIFNR, ORFV020), the chemokine binding protein (CBP, ORFV112), the inhibitor of granulocyte-monocyte colony-stimulating factor and IL-2 (GIF, ORFV117), the viral interleukin 10 homologue (vIL-10, ORFV127), the vascular endothelial growth factor (VEGF-like protein, ORFV132 etc^{[11]}. These viral gene-encoded proteins could also modulate the host's immune response^{[13]}.

The ORFV002 gene is a late viral gene, and (its product) is localized in the nucleus following protein synthesis, inhibiting the NF-κB pathway in the nucleus. It is identified as the first NF-κB inhibitor produced by the ORFV virus.^{[19]}

The ORFV005 gene encodes a hypothetical protein whose mechanism of action is currently unclear.

The ORFV007 gene is about 483 bp and encodes deoxyuridine pyrophosphatase (dUTPase)^{[15]}. Its protein is an important enzyme in the biosynthesis of dNTPs (deoxyribonucleoside triphosphate, dUTPase is involved in the major biosynthesis pathway to dTTP, a type of dNTP). In general, the concentration of dNTPs in normal cells is strictly regulated, which is not conducive to viral replication. In cancer cells, however, there are higher concentrations of dNTPs, which favors viral replication^{[16}]. ORFV007 deletion will restrict viral replication in normal cells, but not in cancer cells, therefore achieving the goal of selective replication of an ORFV in cancer cells.

The ORFV111 gene encodes for a hypothetical protein whose mechanism of action is currently unclear.

The ORFV112 gene encodes the chemokine-binding protein (CBP) and is about 864 bp long. It interferes with the anti-viral mechanism of host immune cells^{[14]}. This protein is similar in structure and function to the CBP-II protein of other pox viruses, which can not only inhibit the migration of DC cells to the inflammatory site, but also prevent DC cells from activating T cells^{[14]}.

Currently, the ORFV viral strains that have been disclosed and used in oncolytic virus research include NZ-2, NZ-7, D1701, NA1/11, etc. In prior art, due to its capacity for inserting a relatively large foreign DNA fragment, an ORFV is generally used as a vector to insert the genes encoding tumor-specific antigens or viral antigens, cytokines, etc., to create a recombinant virus for research.

Patent WO 2012122649A1 disclosed a recombinant ORFV virus with an insertion of the vaccinia virus E3L gene, infecting tumor cells containing specific host-range genes (SPI-1 K1L, C7L, B5R, p28/N1R, E3L etc.), for anti-cancer drug development. Patent CN 108220251A disclosed a recombinant ORFV and its preparation method and use, mainly knocking out the ORFV132 gene (VEGF) of the ORFV NA1/11 strain and inserting the P53-EGFP fusion protein gene into the same site, for cancer drug development. Neither the absence of the ORFV112 gene nor the absence of the ORFV007 gene have been reported in the strains of the two patents said above.

The present invention discloses the mutated ORFV viruses, characterized by the absence of the functionally expressed products of the ORFV112 and/or the ORFV111 gene, wherein absence of the functionally expressed products is due to a complete or partial deletion of the gene ORFV 112 encoding CBP and/or the gene ORFV111 encoding a hypothetical protein. The inventors (of the present disclosure) fortuitously discovered that complete or partial absence of the gene ORFV 112 and/or the gene ORFV111 said above resulted in distinctly enhanced anti-tumor activities by the virus. Furthermore, genome sequence comparison between these mutated type ORFV viruses and all other disclosed ORFV viral strains revealed a complete loss of the dUTPase-encoding gene ORFV 007 of the mutant ORFV viruses said above. Further, the comparison said above also confirmed the complete absence of the ORFV002 and ORFV005 genes of the said mutant ORFV viruses.

Therefore, the mutant Orf viruses in the present disclosure provide a guiding role in anti-cancer drug development for this kind of viruses.

### SUMMARY OF THE INVENTION

Inventors (of the present disclosure) fortuitously discovered that the complete or partial absence of gene ORFV112 (encoding CBP protein) and/or gene ORFV111 (encoding a hypothetical protein, from now on referred to as the hypothetical protein 111), especially the absence of the 5′ end of ORFV112 and the 3′ end of ORFV111, leads to enhanced antitumor activity of the mutant Orf viruses (ORFVs). Furthermore, the inventors also fortuitously discovered that the aforementioned ORFV viruses showed a complete absence of gene ORFV 007. Mutant Orf viruses without functional CBP and/or hypothetical protein 111 can be obtained using standard molecular biology methods (such as molecular cloning, DNA recombination, homologous recombination, PCR, restriction nuclease (digestion), gene knockout, gene silencing, etc.) or emerging molecular biology techniques (e.g., gene editing, etc.).

In one aspect, the present invention discloses a mutant Orf virus characterized by absence of the functionally expressed products of genes ORFV112 and/or ORFV111. In one embodiment, absence of the ORFV112 gene functionally expressed product is caused by complete or partial (e.g., the 5′ end or 3′ end, especially the 5′ end) deletion of the gene ORFV112. In one embodiment, the mature protein sequence of the expressed product of gene ORFV112 is shown in SEQ ID NO:2. In one embodiment, the full sequence of gene ORFV112 is shown in SEQ ID NO:3. In one embodiment, the sequence of SEQ ID NO:3 is completely deleted. In one embodiment, the sequence of SEQ ID NO: 3 is partially deleted, for example, nucleotides 1-1161, 1-1140, 1-538, 539-1139, or 1141-1160 at the 5′ end are deleted. In one embodiment, the partially deleted gene ORFV112 has a sequence of SEQ ID NO: 4 or SEQ ID NO: 57. In one embodiment, absence of the functionally expressed ORFV111 gene product is resulted from partial or complete sequence deletion of the ORFV111 gene (e.g., 5′ end or 3′ end, especially 3′ end). In one embodiment, the sequence of the expressed product of gene ORFV111 is shown in SEQ ID NO: 58. In one embodiment, the full sequence of gene ORFV111 is shown in SEQ ID NO: 59. In one embodiment, the sequence of SEQ ID NO: 59 is completely deleted. In one embodiment, the sequence of SEQ ID NO: 59 is partially deleted. For example, nucleotides 1-793, 1-309, or 310-792 at the 3′ end are deleted. In one embodiment, the partially deleted gene ORFV111 has a sequence of SEQ ID NO: 60. In one embodiment, the sequences of SEQ ID NO: 3 and SEQ ID NO: 59 are completely deleted. In one embodiment, the expressed product is a protein and/or a nucleic acid (especially a functional nucleic acid). In one embodiment, the present invention provides the genomes of the aforementioned ORFV viruses.

In one aspect, the present invention discloses a method to modify Orf viruses, comprising reducing or eliminating the expression and/or activity of the expressed products of gene ORFV112 and/or gene ORFV111. In one embodiment, the method includes complete or partial (e.g., at the 5′ or 3′ end, especially the 5′ end) deletion of the ORFV112 gene. In one embodiment, the mature protein sequence of the expressed product of gene ORFV112 is shown in SEQ ID NO:2. In one embodiment, the full sequence of gene ORFV112 is shown in SEQ ID NO:3. In one embodiment, the method comprises completely deleting the sequence of SEQ ID NO: 3. In one embodiment, the method comprises partially deleting the sequence of gene ORFV112 (SEQ ID NO:3), such as deleting nucleotides 1-1161, 1-1140, 1-538, 539-1139, or 1141-1160 at the 5′ end. In one embodiment, the method generates a partially deleted gene ORFV112 having a sequence of SEQ ID NO: 4 or SEQ ID NO: 57. In one embodiment, the method comprises complete or partial (e.g., the 5′ or 3′ end, especially the 3′ end) deletion of gene ORFV111. In one embodiment, the sequence of the expressed product of gene ORFV111 is shown in SEQ ID NO: 58. In one embodiment, the full sequence of gene ORFV111 is shown in SEQ ID NO: 59. In one embodiment, the method comprises completely deleting the sequence of SEQ ID NO: 59. In one embodiment, the method comprises partially deleting the sequence of SEQ ID NO: 59, for example, deleting nucleotides 1-793, 1-309, or 310-792 at the 3′ end. In one embodiment, the method generates a partially deleted gene ORFV111 of SEQ ID NO: 60. In one embodiment, the method comprises completely deleting the sequences of SEQ ID NO: 3 and SEQ ID NO: 59. In one embodiment, expression is transcription and/or translation. In one aspect, the present invention provides viruses obtained by the method described herein. In one embodiment, this invention provides the genomes of the ORFV viruses described herein. The methods described in the present invention could be carried out by conventional molecular biology techniques (e.g., molecular cloning, DNA recombination, homologous recombination, PCR, restriction nucleases, gene knockout, silencing, etc.) or by emerging molecular biology techniques (e.g., gene editing, etc.).

In another aspect, the present invention provides methods for identifying the aforementioned mutant Orf viruses and/or their genome, in particular methods for detecting gene ORFV112 and/or ORFV111 (its presence or sequence) and/or their expression products (presence and/or activities). The methods described in the present invention could be carried out by detecting the presence and/or activities of proteins and/or the presence and/or sequence of nucleic acids through standard molecular biology techniques. The standard techniques include activity assays, blottings (such as Southern blot, Northern blot, and Western blot), restriction endonuclease (digestion), PCR, electrophoresis (e.g., gel electrophoresis, including protein electrophoresis and nucleic acid electrophoresis, including agarose electrophoresis and PAGE electrophoresis, including reducing and non-reducing electrophoresis), sequencing (including protein sequencing and nucleic acid sequencing), or emerging molecular biology technologies (e.g., next-generation sequencing, etc.). In particular, the present invention provides methods for detecting the integrity of the gene ORFV112 and/or gene ORFV111. In one embodiment, the methods can be carried out by PCR and gel electrophoresis. In one embodiment, the methods can be carried out by hybridization or sequencing.

In another aspect, the present invention provides a method to treat cancers in subjects using the said mutant Orf viruses and/or their genomes. In another aspect, the present disclosure provides the use of the said mutant Orf viruses and/or their genomes to treat cancers in subjects. In another aspect, the present disclosure provides the use of the said mutant Orf viruses and/or their genomes to prepare a drug for treating cancers in subjects. In another aspect, the present disclosure provides the said mutant Orf viruses and/or their genomes for treating caners in subjects. In another aspect, the present disclosure provides the said mutant Orf viruses and/or their genomes for preparing cancer drugs for treating cancers in subjects. In one embodiment, the cancer is a solid tumor. In one embodiment, the said solid tumor is cervical cancer, bladder cancer, liver cancer, ovarian cancer, melanoma, colorectal cancer, lung cancer, breast cancer, gastric cancer, uterine cancer, head and neck cancer, thyroid cancer, esophageal cancer, prostate cancer, pancreatic cancer, sarcoma, a brain tumor, etc. In one embodiment, the said subject is mammal, including rodent (e.g., mice and rats), non-human primate (e.g., cynomolgus monkeys), and human.

### FIGURE DESCRIPTION

FIG. 1 shows the results of agarose gel electrophoresis of PCR products after PCR amplification using ORFV112 gene-specific forward primer and reverse primer (SEQ ID NO: 5 and 6). In the figure, sample 1 is (from) the POV-601-1A1 viral strain with the ORFV112 gene deletion, sample 2 is (from) the POV-601-3F8 viral strain with the complete ORFV112 gene, and M is DNA size marker.

FIG. 2 shows the anti-tumor inhibitory effects of the ORFV112 gene-deleted viral strains (viruses) of POV-601-1A1 and POV-604-1D1 in the MB49 murine bladder cancer model.

FIG. 3 compares the anti-tumor inhibitory effects of the ORFV112 gene-deleted viral strains (viruses), POV-601-1A1 and POV-604-1D1, with the ORFV112 gene-intact strain, POV-601-3F8, in the B16-F10 murine melanoma tumor model.

FIG. 4 shows the influence of the POV-601-1A1 viral strain on mouse body weight under different doses and routes of administration. (i.v., intravenous, s.c., subcutaneous).

FIG. 5 shows immune system changes in mice with human-derived C-33A cervical cancer bilateral tumors following administration of the POV-601-1A1 viral strain. In FIGS. 5A and 5B, the squares represent the results of intratumoral administration on the right side, and the circles represent the results of unadministered mice on the left side. The activation ratio of CD45+ and NK cells in the tumor is increased in mice injected with POV-601-1A1. FIG. 5C shows the activation of NK cells in the blood, and the ratio of activated NK cells is increased by intravenous administration.

FIG. 6 shows a comparison of the gene ORFV112 of the present invention between the complete nucleotide sequences and that of the partial deletion.

FIG. 7 shows the alignment of the complete sequences of the CBP protein of the present invention with that of the CBP proteins of other parapox virus strains, where “3F8” stands for POV-601-3F8 Strain, and “B029” for ORFV Strain B029, “GO” for ORFV Strain GO, “NA11” for ORFV Strain NA1/11, “NZ2” for ORFV Strain NZ2, “NA17” for ORFV Strain NA17, “OV-SA00” for ORFV Strain OV-SA00, “OV-IA82” for ORFV Strain OV-IA82, “SJ1” for ORFV Strain SJ1, “SY17” for ORFV Strain SY17, “OV-NH3_12” for ORFV Strain OV-HN3/12, “NP” for ORFV Strain NP, and “YX” for ORFV Strain YX.

FIG. 8 shows the nucleotide sequence comparison of the ORFV112 gene of the ORFV viral strains POV-601-3F8 of the present invention with that of other parapox virus strains, where “3F8” stands for POV-601-3F8 Strain, “B029” for ORFV Strain B029, “GO” for ORFV Strain GO, “NA11” for ORFV Strain NA1/11, “NZ2” for ORFV Strain NZ2, “NA17” for ORFV Strain NA17, “OV-SA00” for ORFV Strain OV-SA00, “OV-IA82” for ORFV Strain OV-IA82, “SJ1” for ORFV Strain SJ1, “SY17” for ORFV Strain SY17, “OV-NH3_12” for ORFV Strain OV-HN3/12, “NP” for ORFV Strain NP, and “YX” for ORFV Strain YX.

FIG. 9 shows a comparison of the gene ORFV111 of the present invention between the complete nucleotide sequences and that of the partial deletion.

FIG. 10 shows the alignment of the complete sequences of the hypothetical protein 111 of the present invention with that of the hypothetical protein 111 of other parapox virus strains, where “3F8” stands for POV-601-3F8 Strain, “B029” for ORFV Strain B029, “GO” for ORFV Strain GO, “NA11” for ORFV Strain NA1/11, “NZ2” for ORFV Strain NZ2, “NA17” for ORFV Strain NA17, “OV-SA00” for ORFV Strain OV-SA00, “OV-IA82” for ORFV Strain OV-IA82, “SJ1” for ORFV Strain SJ1, “SY17” for ORFV Strain SY17, “OV-NH3_12” for ORFV Strain OV-HN3/12, “NP” for ORFV Strain NP, and “YX” for ORFV Strain YX.

FIG. 11 shows the comparison of the complete sequence of the ORFV111 gene of the present invention with that of the ORFV111 gene of other parapox virus strains, where “3F8” stands for POV-601-3F8 Strain, “B029” for ORFV Strain B029, “GO” for ORFV Strain GO, “NA11 ” for ORFV Strain NA1/11, “NZ2” for ORFV Strain NZ2, “NA17” for ORFV Strain NA17, “OV-SA00” for ORFV Strain OV-SA00, “OV-IA82” for ORFV Strain OV-IA82, “SJ1” for ORFV Strain SJ1, “SY17” for ORFV Strain SY17, “OV-NH3_12” for ORFV Strain OV-HN3/12, “NP” for ORFV Strain NP, and “YX” for ORFV Strain YX.

FIG. 12 shows a comparison of the gene ORFV111 of the present invention between the complete amino acid sequences and that of the partial deletion.

FIG. 13 shows a comparison of the gene ORFV112 of the present invention between the complete amino acid sequences and that of the partial deletion.

FIG. 14 compares the anti-tumor inhibitory effects between the viral strain POV-601-3F8 with a complete ORFV112 gene and the viral strain POV-601-1A1 with a deleted ORFV112 gene in a CT26 mouse colon cancer model.

FIG. 15 compares the anti-tumor inhibitory effects between strains POV-601-1A1 and the v611a in the B16-F10 mouse melanoma tumor model.

FIG. 16 compares the anti-tumor inhibitory effects among strains POV-601-1A1, v615a and v616a in the B16-F10 mouse melanoma tumor model.

FIG. 17 compares the anti-tumor inhibitory effects among strains v601-1A1, v617a and v618a in the B16-F10 mouse melanoma tumor model.

### DETAINED DESCRIPTION OF THE INVENTION

The present invention discloses the mutant Orf viruses and their medicinal compositions for use in cancer treatment.

According to the literature, the virulence genes of a wild-type Orf virus mainly include OVIFNR (orf virus interferon-resistance gene), CBP (chemokine-binding protein), GIF (GM-CSF/IL-2 inhibitory protein), vIL-10 (viral interleukin 10), and VEGF-like protein (vascular endothelial growth factor-like protein), etc^{[11, 13]}. In general, attenuated viruses can be obtained by removing a certain virulence gene using molecular and/or cellular biology methods. For example, patent CN 104878043 B disclosed a method by deleting the virulence gene OVIFNR of the ORFV SHZ1 strain to achieve a rapid reduction in virulence and obtaining an attenuated virus strain, used for preparation of attenuated vaccines.

Mutant Orf viruses disclosed in the present invention were obtained through modification and screening of the original POV-601 viral strain (Deposit No. V201713) deposited in the China Typical Culture Collection Center (CCTCC).

Based on the literature^{[17]}, the inventors' specific primers for the ORFV112 gene were designed. The forward primer (SEQ ID NO: 5) was designed on the coding region sequence of the ORFV111 gene, and the reverse primer (SEQ ID NO: 6) was designed on the coding region sequence of the ORFV112 gene, thereby establishing a PCR-based molecular biology identification technique to check the integrity of the ORFV112 gene of the attenuated POV-601 viruses. Afterwards, African green monkey kidney cells (CV-1) were infected with the parental virus strain POV-601; and the virus-infected cells were collected, sorted into single cell by a flow cytometer, and expanded in culture. Using the said ORFV112 gene-specific primer pairs, extracting viral genomic DNAs (used as the PCR templates) from the expanded and virus-infected cells, the said attenuated ORFV virus strain with a partially deleted ORFV112 gene was obtained by the established molecular biology identification technique, and named it POV-601-1A1. This strain has a 312 bp 5′ deletion and remains 552 bp within the coding region of the ORFV112 gene, and keeps 72 bp in its noncoding region at the 3′ end, a total of 624 bp remaining, see SEQ ID NO: 4). At the same time, POV-601-3F8, a virus having the intact ORFV112 gene with 1162 bp that include both the coding and non-coding regions, has also been obtained. The complete sequence of gene ORFV112 is shown in SEQ ID NO: 3.

When the attenuated POV-601-1A1 viruses were evaluated in an animal tumor efficacy model, it was unexpectedly found that the POV-601-1A1 virus had a better anti-tumor inhibitory effect than POV-601-3F8, the virus with a complete ORFV112 gene.

On the other hand, the inventors (of the present invention) further intentionally used a gene editing technique, in an attempt to knock out all the remaining coding regions of the gene ORFV112 of the POV-601-1A1 virus. In one embodiment, the remaining coding region of gene ORFV112 was completely knocked out (only 22 bp of the 3′ non-coding region were retained, see sequence SEQ ID NO: 57), and (the knocking out result) was confirmed by DNA sequencing. The obtained virus was named POV-604-1D1. In one embodiment, it was unexpectedly found that this virus exhibited a better anti-tumor effect than POV-601-3F8 containing the complete ORFV112 gene, when evaluated in an animal tumor efficacy model. Furthermore, any methods, such as knocking out or changing certain sequences of the coding region and non-coding region of the gene ORFV112, that prevent the expression of the CBP protein, can also achieve the same effect.

The POV-601-1A1 virus was deposited in accordance with Budapest Treaty at China Center for Type Culture Collection (CCTCC) (Wuhan, China) on May 19, 2020, under the CCTCC Deposit No. V202029.

In an embodiment, the mutant Orf viruses of the present disclosure can selectively infect in melanoma cells (B16-F10), bladder cancer cells (MB49), liver cancer cells (Hepa1-6), colon cancer cells (CT26), human cervical cancer cells (C-33A), human ovarian cancer cells (SK-OV-3), etc, and replicate within them.

In an embodiment, the ORFV007 gene of the mutant Orf viruses of the present disclosure was unexpectedly found to be completely deleted, while all the published ORFV strains in NCBI, such as NZ -2, NZ-7, D1701, NA1/11 strains, etc., have the intact gene ORFV007. The ORFV007 gene encodes for the deoxyuridine pyrophosphatase (dUPTase). This protein is an important enzyme for dNTP synthesis. In general, the concentration of dNTP in normal cells is strictly regulated, which is not conducive to viral replication. In cancer cells, however, there are higher concentrations of dNTP, which favors viral replication^{[16]}. The deletion of ORFV007 will restrict viral replication in normal cells, but not in cancer cells, therefore achieving the goal of selective replication of ORFV in cancer cells.

Wild-type ORFV virus can generally be cultured in primary cells from bovine and sheep animal tissues (CN 103952377 A). Patent WO2012122649 A1 first disclosed that ORFV could be proliferated and expanded in the human cervical cancer cell line HeLa. Further, the present invention discloses a method to select out the said mutant Orf viruses using mammalian cells as the host, preferably using the African green monkey kidney cell (line) CV-1 for viral infection and proliferation.

Further, an application of the said viruses in treating individual cancer is provided. The said cancer is any type of a solid tumor. The types of the said solid tumor include: cervical cancer, bladder cancer, liver cancer, ovarian cancer, melanoma, colorectal cancer, lung cancer, breast cancer, gastric cancer, uterine cancer, head and neck cancer, thyroid cancer, esophageal cancer, prostate cancer, pancreatic cancer, sarcoma, brain tumor, etc. Furthermore, the subject is mammal, including rodent and human.

The mechanisms by which oncolytic viruses inhibit tumor growth can generally be classified into 1) replication and amplification in infected tumor cells, lysing cancer cells, and achieving the purpose of oncolysis; 2) Following oncolysis, tumor-specific molecules, such as tumor neoantigens, are released, activating the immune system to mount a systemic attack on remaining cancer cells.

In an embodiment, the mutant Orf viruses of the present disclosure can effectively induce the innate immune response, represented by NK cell activation. In the bilaterally inoculated human C-33A cervical cancer cell model, the proportion of CD45+ and NK cell activation in the tumor and/or blood can be increased through intratumoral and/or intravenous injection of these viruses.
I. General Techniques
Unless otherwise indicated, implementation of the present invention will the employ the conventional techniques of molecular biology (including recombinant techniques), virology, microbiology, cell biology, biochemistry, and immunology, which are within the scope of technology in the field.
II. Definitions
The term “OV” stands for oncolytic virus, the abbreviation of oncolytic virus. The term “solid tumor” refers to a tumor entity composed of multiple cells, as distinguished from blood tumors; it refers to tumors for a variety of cancers including cervical cancer, bladder cancer, liver cancer, ovarian cancer, melanoma, colorectal cancer, lung cancer, breast cancer, gastric cancer, uterine cancer, head and neck cancer, thyroid cancer, esophageal cancer, prostate cancer, pancreatic cancer, sarcoma, brain tumor, etc. Cancers can be at an early or advanced stage.
The term “continuous cell line” refers to a cell population obtained from the primary culture or cell line with special genetic characteristics and biochemical properties or specific markers are called cell lines. A cell line that can be continuously passaged is called a continuous cell line.
The term “CPE” stands for a cytopathic effect, that is, the cellular degeneration following infection of cultured cells by a virus. In an in vitro experiment, cultured cells are inoculated with a cell-killing virus, and cell rounding, necrosis, and detachment can be observed under a microscope after a certain period of time, called cytopathic effects.
The term “Orf”, also known as “contagious pustular dermatitis virus of the sheep” “OVIS”, “ORFV” and “orf virus,” refers to a pox virus that can cause contagious and epitheliotropic diseases mainly in sheep and goats.
The term “effective amount” refers to the quantity needed to achieve the desired therapeutic or preventive effects within the necessary dosage and time; it also refers to the quantity at which the therapeutic beneficial effect of a therapeutic agent outweighs any toxic or harmful consequences.
The term “PBS” refers to phosphate buffer saline.
The term “MOI” stands for multiplicity of infection and refers to the ratio between the number of the viral particles and the total number of target cells (pfu/cell) during a viral infection.
“POV-601-1A1 viral strain”, where “POV” represents “Prajna Oncolytic Virus”, refers to the fact that this viral strain was originated from Suzhou Prajna Biotechnology Co., Ltd. In the internal experimental records of Suzhou Prajna Biotechnology Co., Ltd., POV-601-1A1, v601-1A1, v601-p0-1A1, and 1A1 all represent the same strain. The name of this viral strain is “ORFV mutational type POV-601-1A1” in the CCTCC registry form and belongs to a Orf virus of the parapoxvirus genus of the poxvirus family “POV-601-3F8 viral strain,” where “POV” represents “Prajna Oncolytic Virus”, refers to the fact that this viral strain was originated from Suzhou Prajna Biotechnology Co., Ltd. In the internal experimental records of Suzhou Prajna Biotechnology Co., Ltd., POV-601-1A1, v601-3F8, v601-p0-3F8, and 3F8 all represent the same strain.
The term “POV-604-1D1 viral strain,” where “POV” represents “Prajna Oncolytic Virus”, refers to the fact that this viral strain was originated from Suzhou Prajna Biotechnology Co., Ltd. In the internal experimental records of Suzhou Prajna Biotechnology Co., Ltd., POV-604-1D1, v604-1D1, and 1D1 all represent the same strain.
For “viral storage buffers”: the preparation method is to aspirate 500 mL of PBS (i.e. phosphate buffer, CORNING, catalog number: 21-040-CVR), add 1.25 mL of 1 M MgCl2·6 H2O to make its final concentration 2.48 mM, and then add 2.5 mL of 1M Tris-HCl (pH 9.0) (Sangong Bioengineering (Shanghai) Co., Ltd., catalog number: B548128-0500) to make its final concentration 4.96 mM, mix evenly to obtain the virus storage buffer(the theoretical final concentrations of MgCl2·6 H2O is 2.5 mM and Tris-HCl is 5 mM).
III. Composition and Methods

### 1. ORFV112 Gene Expressed Product, or CBP Protein

The present invention relates to a (natural) CBP. In one embodiment, the (natural) CBP protein has or comprises the sequence shown in SEQ ID NO: 1 or from the same or similar biological source (e.g., strain, species, genus, family) and having at least 90%, 91%, 92%, 93%, 94%, 95%, 96%, 97%, 98%, 99% or 100% identity full-length sequence, or has or comprises a mature protein sequence shown in SEQ ID NO: 2 or from the same or similar biological source (e.g., strain, species, genus, family) having at least 90%, 91%, 92%, 93%, 94%, 95%, 96%, 97%, 98%, 99% or 100% identity of the mature sequence, or is (essentially) composed thereof. In another embodiment, the (natural) CBP proteins have or comprise the sequences shown in SEQ ID NO: 31-42 or are from the same or similar biological source (e.g., strain, species, genus, family) and having at least 90%, 91%, 92%, 93%, 94%, 95%, 96%, 97%, 98%, 99% or 100% full-length sequence identity and the corresponding mature sequence.

The present invention also relates to a mutant type CBP protein. In one embodiment, the mutations are the addition, deletion, or substitution of one or more amino acid residues or any combination thereof, relative to the natural protein sequences. In one embodiment, the function of the mutant type CBP proteins is reduced or lost.

The present invention also relates to a decrease or loss of the expression of the CBP proteins (e.g., natural CBP proteins or mutant type CBP proteins).

For example, the function and/or expression of the CBP proteins are reduced at least 50%, 60%, 70%, 75%, 80%, 90%, 91%, 92%, 93%, 94%, 95%, 96%, 97%, 98%, 99%, to 100%, relative to the natural protein.

### 2. The ORFV112 Gene

The present invention relates to a kind of the (natural) ORFV112 genes. In one embodiment, the (natural) ORFV112 genes encode proteins having or comprising the sequence shown in SEQ ID NO: 1 or from the same or similar biological source (e.g., strain, species, genus, family) and having at least 90%, 91%, 92%, 93%, 94%, 95%, 96%, 97%, 98%, 99% or 100% identity full-length sequence, or having or comprising the sequence shown in SEQ ID NO: 2 or from the same or similar biological source (e.g., strain, species, genus, family) having at least 90%, 91%, 92%, 93%, 94%, 95%, 96%, 97%, 98%, 99% or 100% identity mature protein sequence, or is (essentially) composed thereof. In another embodiment, the (natural) CBP proteins have or comprise the sequences shown in SEQ ID NO: 31-42 or from the same or similar biological source (e.g., strain, species, genus, family) and having at least 90%, 91%, 92%, 93%, 94%, 95%, 96%, 97%, 98%, 99% or 100% identity full-length sequence and the corresponding mature sequence.

The present invention relates to a (natural) ORFV112 gene. In one embodiment, the (natural) ORFV112 gene has or comprises the sequence shown in SEQ ID NO: 3 (or its coding region), encodes the same amino acid sequence, or come from the same or similar biological source (e.g., strain, species, genus, family) and has at least 75%, 80%, 85%, 90%, 91%, 92%, 93%, 94%, 95%, 96%, 97%, 98%, 99% or 100% identity nucleotide sequence, or is (essentially) composed thereof. The sequence shown in SEQ ID NO: 3 contains 1162 nucleotides, of which nucleotides 1-226 belong to the 5′ non-coding region, nucleotides 227-1090 cover the protein-coding region, and nucleotides 1091-1162 belong to the 3′ non-coding region. In another embodiment, the (natural) ORFV112 genes have or comprise the nucleotide sequences shown in SEQ ID NO: 43-54 (or its coding region), encode the same amino acid sequence, or come from the same or similar biological source (e.g., strain, species, genus, family) and have at least 75%, 80%, 85%, 90%, 91%, 92%, 93%, 94%, 95%, 96%, 97%, 98%, 99% or 100% identity nucleotide sequences.

The present invention also relates to a type of the mutant ORFV112 genes. In one embodiment, the mutations are the addition, deletion, or substitution of one or more nucleotide or any combination thereof, relative to the natural nucleotide sequence. In one embodiment, the mutant type ORFV112 gene is the ORFV112 gene with a complete deletion. The full-deletion type ORFV112 gene refers to the absence of the entire ORFV112 gene. In one embodiment, the mutant type ORFV112 gene is a partially deleted ORFV112 gene. Partial deletion type ORFV112 gene lacks one or more but not all nucleotides of the ORFV112 gene. In one embodiment, the partial deletion type ORFV112 gene lacks one or more nucleotides in the 5′ non-coding region, the coding region and/or the 3′ non-coding region, or any combination thereof. In one embodiment, the partial deletion type ORFV112 gene is a 5′ terminal deletion type ORFV112 gene, that is, one or more nucleotides at the 5′ end are missing, such as missing the entire or part of the 5′ non-translated region, missing the entire 5′ non-translated region and all or part of the coding region, or missing the entire 5′ non-translated region, the entire coding region, and all or part of the 3′ non-translated region. In one embodiment, the ORFV112 gene deficiency extends to the upstream region (the ORFV111/112 intergenic region, if present, and/or the ORFV111 gene, specifically its 3′ end) and/or the downstream region (the ORFV112/113 intergenic region, if present, and/or the ORFV113 gene, especially 5′ end).

In the case where the natural ORFV112 gene has or comprises a nucleotide sequence (or its coding region) shown in SEQ ID NO: 3 or is (essentially) composed thereof, in one embodiment, the partial deletion type ORFV112 gene lacks one or more nucleotides of the sequence shown in SEQ ID NO: 3, such as the 1-1161 nucleotides. In the case where the natural ORFV112 gene has or comprises a nucleotide sequence (or its coding region) shown in SEQ ID NO: 3 or is (essentially) composed thereof, in one embodiment, the 5′ end partial deletion type ORFV112 gene lacks one or more nucleotides of the sequence shown in SEQ ID NO: 3, in particular the 5′ end 1-1161, 1-1140, 1-538, 539-1139, or 1141-1160 nucleotides. In one embodiment, the 5′ end partial deletion type ORFV112 gene has or comprises the nucleotide sequence (or its coding region) shown in SEQ ID NO: 4 or SEQ ID NO: 57, or is (essentially) composed thereof. In the case where the natural ORFV112 gene has or comprises the nucleotide sequences (or its coding region) shown in SEQ ID NO: 43-54, or is (essentially) composed thereof, in one embodiment, the 5′ end deletion type ORFV112 gene lacks one or more nucleotides at the 5′ end of the sequences shown in SEQ ID NO: 43-54, especially one or more nucleotides of a segment corresponding to 1140 or 538 nucleotides at the 5′ end of SEQ ID NO: 3. Due to the deletion of the 5′ non-translated region and/or the initiation codon, these deletion type genes are unable to express proteins.

In one embodiment, the mutation type ORFV112 gene of the present invention results in a decrease or loss of the functional CBP protein, including a decrease and loss in expression and/or activity of CBP. For example, the decrease is of at least 50%, 60%, 70%, 75%, 80%, 90%, 91%, 92%, 93%, 94%, 95%, 96%, 97%, 98%, 99%, to 100%, while compared to the natural function and/or expression.

### 3. The Expressed Product of the ORFV111 Gene, the Hypothetical Protein 111

The present invention relates to a (natural) hypothetical protein 111. In one embodiment, the (natural) hypothetical protein 111, having or comprising the sequence shown in SEQ ID NO: 58 or from the same or a similar biological source (e.g. strain, species, genus, family) and having at least 90%, 91%, 92%, 93%, 94%, 95%, 96%, 97%, 98%, 99% or 100% identity sequence, or is (essentially) composed thereof. In another embodiment, the (natural) hypothetical proteins 111 have or comprise the sequences shown in SEQ ID NO: 61-72 or are from the same or a similar biological source (e.g. strain, species, genus, family) and have at least 90%, 91%, 92%, 93%, 94%, 95%, 96%, 97%, 98%, 99% or 100% identity sequence.

The present invention also relates to a mutant type hypothetical protein 111. In one embodiment, the mutations are the addition, deletion, or substitution of one or more amino acid or any combination thereof relative to the natural sequences. In one embodiment, the function of the mutant type hypothetical protein 111 is reduced or lost.

The present invention also relates to a decrease or loss of the expression of the hypothetical protein 111 (e.g., the natural hypothetical protein 111 or the mutant type hypothetical protein 111).

For example, the function and/or expression of the hypothetical protein 111 are reduced at least 50%, 60%, 70%, 75%, 80%, 90%, 91%, 92%, 93%, 94%, 95%, 96%, 97%, 98%, 99%, to 100%, relative to the natural protein.

### 4. The ORFV111 Gene

The present invention relates to a (natural) ORFV111 gene. In one embodiment, the (natural) ORFV111 genes encode proteins having or comprising the sequence shown in SEQ ID NO: 58 or from the same or similar biological source (e.g., strain, species, genus, family) and having at least 90%, 91%, 92%, 93%, 94%, 95%, 96%, 97%, 98%, 99% or 100% identity sequence, or the hypothetical protein 111 is (essentially) composed thereof. In another embodiment, the (natural) hypothetical protein 111 has or comprise the sequences shown in SEQ ID NO: 61-72 or from the same or similar biological sources (e.g., strain, species, genus, family) and has at least 90%, 91%, 92%, 93%, 94%, 95%, 96%, 97%, 98%, 99% or 100% identity sequence.

The present invention relates to a (natural) ORFV111 gene. In one embodiment, the (natural) ORFV111 gene has or comprises the sequence shown in SEQ ID NO: 59 and encodes the same amino acid sequence or come from the same or similar biological sources (e.g. strain, species, genus, family) and has at least 75%, 80%, 85%, 90%, 91%, 92%, 93%, 94%, 95%, 96%, 97%, 98%, 99% or 100% identity nucleotide sequences, or is (essentially) composed thereof. The sequence shown in SEQ ID NO: 59 contains 794 nucleotides, of which nucleotides 1-28 belong to the 5′ non-coding region, nucleotides 29-568 cover the protein-coding region, and nucleotides 569-794 belong to the 3′ non-coding region. In another embodiment, the (natural) ORFV111 genes have or comprise the nucleotide sequences shown in SEQ ID NO: 73-84 (or the coding region), encode the same amino acid sequence, or come from the same or similar biological sources (e.g., strain, species, genus, family) and have at least 75%, 80%, 85%, 90%, 91%, 92%, 93%, 94% %, 95%, 96%, 97%, 98%, 99% or 100% identity nucleotide sequences.

The present invention relates to a type of mutant ORFV111 genes. In one embodiment, the mutations are the addition, deletion, or substitution of one or more nucleotides or any combination thereof, relative to the natural nucleotide sequence. In one embodiment, the mutant type ORFV111 gene is the ORFV111 gene with a complete deletion. The full-deletion type ORFV111 gene refers to the absence of the entire ORFV111 gene. In one embodiment, the mutant type ORFV111 gene is a partially deleted ORFV111 gene. Partial deletion type ORFV111 gene lacks one or more but not all nucleotides of the ORFV111 gene. In one embodiment, the partial deletion type ORFV111 gene lacks one or more nucleotides in the 3′ non-coding region, the coding region and/or the 5′ non-coding region, or any combination thereof. In one embodiment, the partial deletion type ORFV111 gene is a 3′ terminal deletion type ORFV111 gene, that is, one or more nucleotides at the 3′ end are missing, such as missing the entire or part of the 3′ non-translated region, missing the entire 3′ non-translated region and all or part of the coding region, or missing the entire 3′ non-translated region, the entire coding region and all or part of the 5′ non-translated region. In one embodiment, the ORFV111 gene deficiency extends to the downstream region (the ORFV111/112 intergenic region, if present, and/or the ORFV112 gene, specifically its 5′ end) and/or the upstream region (the ORFV110/111 intergenic region, if present, and/or the ORFV110 gene, especially its 3′ end).

In the case where the natural ORFV111 gene has or comprises a nucleotide sequence (or its coding region) shown in SEQ ID NO: 59 or is (essentially) composed thereof, in one embodiment, the partial deletion type ORFV111 gene lacks one or more nucleotides of the sequence shown in SEQ ID NO: 59, such as the 1-793 nucleotides. In the case where the natural ORFV111 gene has or comprises a nucleotide sequence (or its coding region) shown in SEQ ID NO: 59 or is (essentially) composed thereof, in one embodiment, the 3′ end partial deletion type ORFV111 gene lacks one or more nucleotides at the 3′ end of the sequence shown in SEQ ID NO: 59, especially the 3′ end 1-793; 1-309 or 310-792 nucleotides. In one embodiment, the 3′ end deletion type ORFV111 gene has or comprises the nucleotide sequence (or its coding region) shown in SEQ ID NO: 60 or is (essentially) composed thereof. In the case where the natural ORFV111 gene has or comprises the nucleotide sequences (or its coding region) shown in SEQ ID NO: 73-84 or is (essentially) composed thereof, in one embodiment, the 3′ terminal deletion type ORFV111 gene lacks one or more nucleotides at the 3′ end of the sequence shown in SEQ ID NO: 73-84, especially one or more nucleotides of a segment corresponding to 309 nucleotides at the 3′ end of SEQ ID NO: 59. Due to the deletion of the 3′ non-translated region, these deletion type genes are unable to express proteins.

In one embodiment, mutations of the ORFV111 gene of the present invention results in a decrease or loss of functional hypothetical protein 111, including a decrease or loss of the expression and/or activity of hypothetical protein 111. For example, the decrease is of at least 50%, 60%, 70%, 75%, 80%, 90%, 91%, 92%, 93%, 94%, 95%, 96%, 97%, 98%, 99%, to 100%, while compared to the natural function and/or expression.

### 5. The ORFV Viral Genome

The present invention relates to an ORFV virus genome. In one embodiment, the ORFV viral genome has the said above ORFV112 gene and/or ORFV111 gene, including the natural ORFV112 gene and/or ORFV111 gene and mutated types of the ORFV112 gene and/or ORFV111 gene. The present invention particularly relates to an ORFV virus genome with ORFV112 gene and/or ORFV111 gene deficiency. In one embodiment, the ORFV viral genome with ORFV112 gene and/or ORFV111 gene-deficient comprises the said mutant type ORFV112 gene and/or ORFV111 gene. In one embodiment, the ORFV viral genome (e.g., ORFV virus genome with ORFV112 gene and/or ORFV111 gene-deficient) completely loses the ORFV007 gene. In one embodiment, the 3′ non-coding region of the ORFV111 gene overlaps with the 5′ non-coding region of the ORFV112 gene.

### 6. The Orf Virus

The present invention relates to a type of ORFV viruses. In one embodiment, the ORFV virus has the CBP protein and/or hypothetical protein 111 mentioned above, including the natural CBP protein and/or hypothetical protein 111 and the mutant type of the CBP protein and/or hypothetical protein 111. The present invention in particular relates to the ORFV virus with deficient CBP protein and/or hypothetical protein 111. In one embodiment, the ORFV virus with a deficient CBP protein and/or hypothetical protein 111 lowers or loses the functional CBP protein and/or hypothetical protein 111. Reduction or loss of the functional CBP protein and/or hypothetical protein 111 may result from reduction or loss of the expression and/or activity of the CBP protein and/or hypothetical protein 111.

The present invention relates to a type of ORFV viruses. In one embodiment, the ORFV virus contains the said ORFV112 gene and/or ORFV111 gene, including the natural ORFV112 gene and/or ORFV111 gene and the mutant type ORFV112 gene and/or ORFV111 gene. In one embodiment, the ORFV virus contains the ORFV viral genome said above, including the ORFV viral genome with deficient ORFV112 gene and/or the ORFV111 gene. In particular, the present invention relates to a type of ORFV virus with deficient CBP protein and/or hypothetical protein 111 , comprising the previously said mutated type ORFV112 gene and/or ORFV111 gene or the previously said ORFV112 gene and/or ORFV111 gene-defective ORFV viral genome.

In one embodiment, the ORFV virus (e.g. the ORFV viruses with CBP protein and/or hypothetical protein 111 deficiency) lacks the functional transcription and/or translational products of the gene ORFV007, including the loss of expression and/or activity.

### 7. Methods for Modifying ORFV Virus

The present invention relates to a type of methods for modifying the ORFV virus genome or the ORFV virus. In one embodiment, the methods comprise mutating the ORFV112 gene and/or the ORFV111 gene within the ORFV viral genome. In one embodiment, the methods comprise adding, deleting, or replacing, particularly deleting one or more nucleotides of the ORFV112 gene and/or ORFV111 gene, particularly (deleting) one or more nucleotides at the 5′ end of the ORFV112 gene and/or one or more nucleotides at the 3′ end of the ORFV111 gene.

In the case where the natural ORFV112 gene has or comprises a nucleotide sequence (or its coding region) shown in SEQ ID NO: 3 or is (essentially) composed thereof, in one embodiment, the methods comprise deleting one or more nucleotides of the sequence shown in SEQ ID NO: 3, such as 1-1162 nucleotides. In the case where the natural ORFV112 gene has or comprises a nucleotide sequence (or its coding region) shown in SEQ ID NO: 3 or is (essentially) composed thereof, in one embodiment, the methods comprise deleting one or more nucleotides at the 5′ end of the sequence shown in SEQ ID NO:3, especially 1-1161, 1-1140, 1-538, 539-1139, or 1141-1160 nucleotides at the 5′ end, such as 538 or 1140 nucleotides. In the case where the natural ORFV112 gene has or comprises a nucleotide sequence (or its coding region) shown in SEQ ID NO: 3 or is (essentially) composed thereof, in one embodiment, the methods comprise the complete deletion of the entire sequence shown in SEQ ID NO: 3 or the deletion of 5′ terminal 1140 or 538 nucleotides of SEQ ID NO: 3. In the case where the natural ORFV112 gene has or comprises a nucleotide sequence (or its coding region) shown in SEQ ID NO: 3 or is (essentially) composed thereof, in one embodiment, the methods comprise completely deleting the said nucleotide sequences or deleting a region of the said nucleotide sequence corresponding to 1140 or 538 nucleotides at the 5′ end of SEQ ID NO: 3. In the case where the natural ORFV111 gene has or comprises a nucleotide sequence (or its coding region) shown in SEQ ID NO: 59 or is (essentially) composed thereof, in one embodiment, the methods comprise deleting one or more nucleotides of the sequence shown in SEQ ID NO: 59, such as 1-794 nucleotides. In the case where the natural ORFV111 gene has or comprises a nucleotide sequence (or its coding region) shown in SEQ ID NO: 59 or is (essentially) composed thereof, in one embodiment, the methods comprise deleting one or more nucleotides at the 3′ end of the sequence shown in SEQ ID NO: 59, especially 1-793, 1-309 or 310-792 nucleotides at the 3′ end, such as 309 nucleotides. In the case where the natural ORFV111 gene has or comprises a nucleotide sequence (or its coding region) shown in SEQ ID NO: 59 or is (essentially) composed thereof, in one embodiment, the methods comprise completely deleting SEQ ID NO: 59 or deleting the 309 nucleotides at the 3′ end of the sequence shown in SEQ ID NO: 59. In the case where the natural ORFV111 gene has or comprises a nucleotide sequence (or its coding region) shown in SEQ ID NO: 59 or is (essentially) composed thereof, in one embodiment, the methods comprise completely deleting the said nucleotide sequences or deleting a region of the said nucleotide sequence corresponding to 309 nucleotides at the 3′ end of SEQ ID NO: 59. In the case where the natural ORFV111 gene and the natural ORFV112 gene have or comprise other nucleotide sequences (or its coding regions) or are (essentially) composed thereof, in one embodiment, the methods comprise completely deleting the sequences shown in SEQ ID NO: 3 and SEQ ID NO: 59. In one embodiment, the methods also comprise mutating (e.g. deletion, including complete deletion or partial deletion) the ORFV007 gene in the ORFV viral genome (especially ORFV112 gene and/or ORFV111 gene-deficient ORFV viral genome). In one embodiment, the 3′ non-coding region of the ORFV111 gene overlaps with the 5′ non-coding region of the ORFV112 gene. The methods of the present invention can be carried out by conventional molecular biology techniques (such as molecular cloning, DNA recombination, homologous recombination, PCR, restriction nuclease (digestion), gene knockout, silencing, etc.) or emerging molecular biology techniques (such as gene editing, etc.). The present invention relates to obtaining the ORFV viral genome (such as ORFV112 gene and/or ORFV111 gene-deficient ORFV viral genome) and ORFV virus (such as CBP protein and/or hypothetical protein 111-deficient ORFV virus) through the utilization of the said methods.

### 8. Methods for Identifying ORFV Viral Genomes and ORFV Viruses

The present invention relates to a method for identifying the ORFV viral genome or the ORFV virus, in particular the method for detecting the (presence or activity) of the CBP protein and/or hypothetical protein 111 (especially mutant type CBP protein and/or hypothetical protein 111) and/or the (presence or sequence) of the ORFV112 and/or ORFV111 genes (especially mutant type ORFV112 and/or ORFV111 genes). The methods of the present invention can be carried out by the techniques for detecting the presence or activity of a protein or the presence or sequence of a nucleic acid, and these conventional molecular biology techniques include activity assay, hybridization (such as Southern hybridization, Northern hybridization, or Western hybridization), restriction nucleases, PCR, electrophoresis (such as gel electrophoresis, including protein electrophoresis and nucleic acid electrophoresis (agarose electrophoresis and PAGE electrophoresis, reducing and non-reducing electrophoresis), sequencing (including protein sequencing and nucleic acid sequencing), etc.) or emerging molecular biology techniques (such as next-generation sequencing, etc.). The present invention especially provides methods for detecting the integrity (or length) of gene ORFV112 and/or ORFV111. In one embodiment, the method is performed by PCR and gel electrophoresis. In one embodiment, the method can be performed by nucleic acid hybridization or sequencing.

The design of primers and probes is within the competence of those skilled in the art. Those skilled in the art know how to select primers and probes on the target area of the target sequence. For example, to detect the integrity of the gene ORFV112, on the one hand, the target region of the upstream primer can be set, but not limited to, at the 5′ end of the gene ORFV112 or can extend upstream, for example, into the ORFV111/112 intergenic region, if any, or into gene ORFV111 (especially the 3′ end of gene ORFV111, e.g., 3′ non-translated region); on the other hand, the target region of the downstream primer can also be set at the 3′ end of the gene ORFV112 or can extend downstream, for example, into the ORFV112/113 intergenic region, if any, or into gene ORFV113 (specifically the 5′ end of the gene ORFV113, e.g., the 5′ non-translated region). Similarly, primer/probes can be designed based on similar principles.

### 9. Uses of the ORFV Viral Genome and ORFV Virus

The present invention relates to a pharmaceutical composition, which comprises a certain amount, especially an effective amount, such as a therapeutically effective amount or a preventive effective amount, of the ORFV viral genomes (such as the ORFV112 gene and/or ORFV111 gene-deficient ORFV viral genome) and/or the ORFV viruses (e.g., CBP protein and/or hypothetical protein 111-deficient ORFV viruses) of the present invention. In one embodiment, the pharmaceutical composition comprises a pharmaceutically acceptable carrier.

In one embodiment, the said composition is in a form of powder, solution, transdermal patch, ointment, or suppository. In one embodiment, the composition is administered through intravenous, intratumoral, intramuscular, subcutaneous, rectal, vaginal, or intraperitoneal routes.

The present invention relates to a method for treating a disease or delaying disease progression in subjects, which comprises administering a certain amount, particularly an effective amount, such as a therapeutically effective amount or a preventively effective amount of the ORFV viral genome (such as the ORFV112 gene and/or ORFV111 gene-deficient ORFV virus genome) and/or the ORFV virus (such as the CBP protein and/or hypothetical protein 111-deficient ORFV virus) of the present invention.

The present invention relates to a use of a certain amount, especially an effective amount, such as a therapeutically effective amount or a preventive effective amount of the ORFV viral genome (such as ORFV112 gene and/or ORFV111 gene-deficient ORFV viral genome) and/or ORFV virus (such as CBP protein and/or hypothetical protein 111-deficient ORFV virus) of the present invention in the preparation of drugs. In one embodiment, the said drug is used to treat the disease or delay disease progression in the subject.

The present invention relates to a certain amount, especially an effective amount, such as a therapeutically effective amount or a preventive effective amount of the ORFV viral genome (such as ORFV112 gene and/or ORFV111 gene-deficient ORFV viral genome) and/or ORFV virus (such as CBP protein and/or hypothetical protein 111-deficient ORFV virus) of the present invention, which are used to treat the disease or delay disease progression in the subject.

In one embodiment, the said disease is cancer. In one embodiment, the said cancer is a solid tumor. In one embodiment, the said solid tumor is cervical cancer, bladder cancer, liver cancer, ovarian cancer, melanoma, colorectal cancer, lung cancer, breast cancer, gastric cancer, uterine cancer, head and neck cancer, thyroid cancer, esophageal cancer, prostate cancer, pancreatic cancer, sarcoma, brain tumor, etc.

In one embodiment, the said subject is a mammal, including rodent (e.g., mouse and rat), non-human primate (e.g., cynomolgus monkey), and human.

### EXAMPLES

### Example 1: Screening and Purification Method of the ORFV Mutants (Strains)

Virus source: POV-601 Viral Strain (China Center for Type Culture Collection (CCTCC), Deposit No. V201713)

Host cell source: African green monkey kidney cell CV-1 (National Collection of Authenticated Cell Cultures/Shanghai Institutes for Biological Sciences, CAS)

Flow cytometry cell sorting and single clone picking methods:

1) Infected the African green monkey kidney CV-1 cells with the POV-601 virus strain, followed by sorting of the viral infected cells with a flow cytometer (BD). Then, seeded the cells into 96-well plates, one cell each well;

2) Transferred the sorted 96-well plates into a 37° C., 5% CO2 incubator (Thermo, 160i) for culturing, and observed the infection daily;

3) When virus-infected cells displayed enough CPE, collected and cryopreserved the virus;

4) Extracted the viral genomic DNA from each collected monoclonal virus respectively using the QuickExtract™ DNA Extraction Solution (Lucigen, Cat#: QE09050);

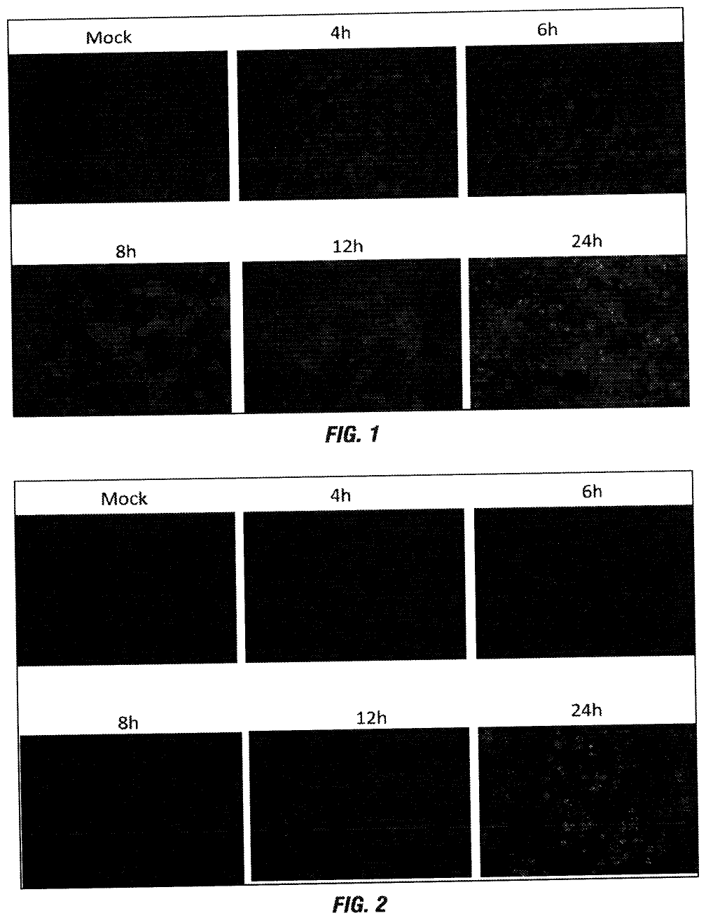

5) Using the designed ORFV112 gene-specific forward and reverse primers (SEQ ID NO: 5 and 6) and the genomic DNA extracted from a single viral clone as the template, target fragment amplification and agarose gel electrophoresis were performed respectively. FIG. 1 shows the electrophoresis results of the monoclonal viral strains(mutants) POV-601-1A1 and POV-601-3F8, and their band sizes are about 500bp and 1000bp respectively. Meanwhile, perform PCR amplification, using the ORFV112 gene-specific forward and reverse primers (SEQ ID NO: 5 and 6) and the POV-601-3F8 forward and reverse primers (SEQ ID NO: 55 and 56) respectively, and Sanger sequencing with the PCR products, confirming that the ORFV112 gene of POV-601-1A1 missed 538 bases at the 5′ end, which contain 226 bases of the 5′ end non-coding and 312 bases of the 5′ end of protein-coding region (see SEQ ID NO: 4). POV-601-3F8 has a complete ORFV112 gene sequence (see SEQ ID NO: 3).

The parameters of the PCR program: 94° C. Pre-denaturation 5 min; 94° C. Denaturation 30 s, 62° C. Primer Annealing 30 s, 68° C. Extension 1 min, 30 Cycles; 68° C. Final Extension 7 min.

### Example 2: Confirmation of the Species of the ORFV Mutant (Strain)

The viral genome (DNA) extracted from the POV-601-1A1 viral strain (using the kit (Mouse Tail Genomic DNA Kit, Cat#: CW2094S)) was sent to a third-party sequencing company for next-generation sequencing (Genewiz Inc., Suzhou). Sequencing results were assembled and analyzed. The complete B2L gene^{[21]} (ORFV011) was compared to other published sequences using BLAST, the one showing higher similarity is the OV/HLJ/04 (strain), with a 99.91% similarity. Therefore, it was confirmed that the isolated virus was a ORFV (virus).

| | | Query | Per. | |
|---|---|---|---|---|
| No. | Description | Cover | Ident | Accession |
| 1 | Orf virus strain OV/HLJ/04 | 99% | 99.91% | KU523790.1 |
| 2 | Orf virus isolate YL-3 | 100% | 99.82% | KF772211.1 |
| 3 | Orf virus strain China Vaccine | 100% | 99.82% | JQ904789.1 |
| 4 | Orf virus strain | 100% | 99.74% | KU194469.1 |
| | ORFV/Shaanxi/2015/China | | | |
| 5 | Orf virus strain XD | 100% | 98.94% | KM675392.1 |

### Example 3: Construction of the ORF Mutants

Source of the viral strain: the POV-601-1A1 viral strain (China Center for Type Culture Collectio (CCTCC), Deposit No: V202029)

Source of the host cell: African Green Monkey Kidney Cell CV-1 (ATCC, No. CCL70™)

The method for constructing recombinant ORFV, mainly consisting of the following steps:

1) Using the specifically designed primers with the HindIII and EcoRI restriction endonuclease sites at the ends (SEQ ID NO: 7-12), the flanking sequence of the ORFV112 gene (the left arm and right arm) and the EGFP report gene were cloned (amplified) by PCR;
2) Using HindIII and EcoRI to digest the pUC19 plasmid;
3) The PCR products in step 1) were ligated with the enzyme-cut pUC19 plasmid to obtain the CBP shuttle plasmid;
4) Using the ligated CBP shuttle plasmid obtained from 3) as a template, and the specific primers (SEQ ID NO: 13-14) with the EcoRI and AgeI restriction endonuclease sites at the ends, a linearized CBP shuttle vector was obtained by PCR amplification;
5) A new Cas9 plasmid vector (fragment) without a nuclear localization signal (NLS) and a new px330 plasmid vector backbone (fragment) were amplified by PCR amplification using the px330 plasmid vector as a template and the specific primers (SEQ ID NO: 15-18) with the EcoRI and AgeI restriction endonuclease sites at their ends respectively;
6) Digested the new Cas9 fragment and px330 backbone from step 5 with the EcoRI and AgeI enzymes and ligated them by the T4 DNA ligase. The ligated products were transformed into a host bacteria strain DH5α to obtain positive (desired) clones. Individual clones were expanded in culture and their plasmid (DNA) was extracted. After Sanger sequencing verification, the recombinant plasmid without a nuclear localization signal, px330-ΔNLS, was obtained;
7) Synthesized the CBP gRNAs, digested the px330-ΔNLS recombinant plasmid with the BbSI enzyme, and then ligated the CBP gRNAs and the digested px330-ΔNLS. The ligated products were transformed into a host bacteria strain DH5α to obtain positive (desired) clones. Individual (positive) clones were expanded in culture, its plasmid (DNA) was extracted, and the px330-ΔNLS-CBP gRNA expression plasmid was obtained;
8) Transfected the selected parapoxvirus host cell CV-1 with the obtained px330-ΔNLS-CBP gRNA plasmid, followed by infecting the transfected CV-1 cells with the POV-601-1A1 viral strain. Transfected the linear CBP shuttle vector obtained in step (4) into CV-1 cells that have been transfected with px330-ΔNLS-CBP gRNA plasmid and infected with the POV-601-1A1 virus;
9) Enriched and cultured the target cells having the EGFP fluorescent virus, and then sorted the cells with a flow cytometer into 96-well plates (1 cell per well), followed by culture expansion. Harvested the target cells from the well containing rich EGFP fluoresce virus, extracted the viral genomic DNA and used the PCR method for identification (the primers, SEQ ID NO: 19-30). Infected CV-1 cells with the virus showing the target bands again, and culturally expanded (the infected cells);
10) Through multiple rounds of virus inoculation, sorting, virus collection, and PCR identification, a pure monoclonal virus with a complete deletion of the ORFV112 gene was obtained, named as POV-604-1D1.

### Example 4: Culture of the ORFV Mutants

Source of the host cell: African Green Monkey Kidney Cell CV-1 (ATCC No. CCL70™)
Source of the viral strains (mutants): POV-601, POV-601-1A1, POV-604-1D1 and POV-601-3F8 viral strains (mutants)

Viral infection of the cell:

1) Subcultured CV-1 cells according to the required amount. Cells in flasks could be infected when about 90% of cell confluence was reached;
2) The viral solution was appropriately diluted with a 2% FBS/DMEM complete medium
3) Discarded the used media from flasks and replenished with appropriate amount of the fresh 2% FBS/DMEM complete medium.
4) Added an already diluted viral solution at a MOI of 0.5. Gently shook the flasks to let the virus distribute evenly, labelled the flasks and transferred them into a 37° C., 5% CO2 incubator;

Cryopreservation of the virus-infected cells

1) When more than 90% of the virus-infected cells displayed CPE, the virus could be harvested;
2) Took out the virus-infected cells from the cell culture incubator, collected the supernatant; trypsinized the adherent cells and combined them with the collected supernatant;
3) Centrifuged (the cell/supernatant mix from 2)) at 3000 rpm, at 4° C. for 10min;
4) Rinsed (the pellet from 3)) with PBS once;
5) Resuspended the pellet (cell) with an appropriate amount of PBS;
6) Transferred (the suspension) into a −80° C. freezer for storage;

Cell lysis to release the virus

1) Transferred the virus-infected cells from −80° C. to a 37° C. water bath for thawing;
2) Sonicated the cells three times (Thermo, Model # FB120): Set “Amplitude” to 100%, “Time” to 1 min;
3) Added Benzonase (25 U/ml), gently mixed, and incubated at 37° C. for 30 min;
4) Centrifuged at 1300 rpm for 10 min;
5) Collected the viral supernatant and transferred it to −80° C. for storage.

### Example 5: Evaluation of Antitumor Activity of the ORFV Mutants in A Mouse Bladder Cancer Model

A. Experimental design

TABLE 1

| | | | Route of | |
|---|---|---|---|---|
| | Animal | Virus | Adminis- | Dosage and |
| Group | Number | Titer | tration | Frequency |
| Control (PBS) | 6 | / | Intra-tumoral | First injection |
| POV-601-1A1 | 6 | 2.07*10⁸ | injection | upon the day of |
| | | pfu/ml | | grouping, |
| POV-604-1D1 | 6 | 1.9*10⁸ | | followed by one |
| | | pfu/ml | | injection every |
| | | | | 72 h, 40 μL/mouse |

B. Experimental animals: Male C57BL/6 mice, 6W, (Zhejiang Vital River Laboratory Animal Technology Co., Ltd.)

### C. Procedures:

Implanted a male C57BL/6 mouse subcutaneously on the right back with MB49 cells (GuangZhou Jennio Biotech Co., Ltd) resuspended in PBS; the cell density was 2×10⁶/ml and the inoculation dose was 0.1 ml/mice. The date of cell implantation was defined as Day 0. When the average volume of the tumors in the control group reached about 100mm³, mice were randomly assigned to different groups based on the tumor size. Injection was performed according to Table 1, and all the mice were euthanized on day 30;

Weighed mice and measured tumor volumes twice a week throughout the entire study. The tumor volume was measured using a vernier caliper (Mahr GmbH, Model 16ER), and the results are shown in FIG. 2.

Calculated the tumor volume by the following formula: tumor volume (mm³)=a*b²/2, wherein a is the tumor length (mm), b is the tumor width (mm). Length and width were measured vertically.

Calculated the relative mean of tumor volumes by the following formula: RTV (Relative Tumor Volume)=Vₜ/V₀, wherein, V₀ is the average tumor volume measured during group assignment, and Vₜ is the average tumor volume at each measurement. Relative tumor proliferation rate T/C (%)=T_{RTV}/C_{RTV}*100%. (T_{RTV}: treatment group RTV; C_{RTV}: control group RTV).

Tumor growth inhibition rate TGI%=(1−T/C)×100%

ANOVA for statistical analysis: ANOVA analysis was used to compare whether there was a significant difference in tumor volumes between the experimental and control groups. All the data were analyzed with SPSS 17, and the statistical significance was defined as P<0.05.

D. The result

TABLE 2

| | Average | | TGI | |
|---|---|---|---|---|
| | Tumor | TGI | Analysis | P-value |
| Day 30 | Size | (%) | (TGI > 60%) | (ANOVA) |
| Control (PBS) | 3066.11 | — | — | — |
| POV-601-1A1 | 558.53 | 82 | 5/6 | 0.008** |
| POV-604-1D1 | 102.85 | 100 | 6/6 | 0.002** |

### Example 6: Evaluation of Antitumor Activity of the ORFV Mutants in a Mouse Melanoma Model

A. Experimental design

TABLE 3

| | | | Route of | |
|---|---|---|---|---|
| | Number | Virus | Adminis- | Dosage |
| Group | of Mice | Titer | tration | and Frequency |
| Control (PBS) | 5 | / | Intratumoral | First injection |
| POV-604-1D1 | 5 | 1.90*10⁸ | injection | upon the day of |
| | | pfu/ml | | grouping, |
| POV-601-3F8 | 5 | 4.31*10⁸ | | followed by one |
| | | pfu/ml | | injection every |
| POV-601-1A1 | 5 | 2.98*10⁸ | | 72 h, 40 μL/mouse |
| | | pfu/ml | | |

B. Experimental animals: Female C57BL/6 mice, 6-8W, Zhejiang Vital River Laboratory Animal Technology Co., Ltd).

### C. Procedures

Implanted female C57BL/6 mice subcutaneously on their right back with B16-F10 cells (Cell Bank of Type Culture Collection Committee of Chinese Academy of Sciences) resuspended in PBS; the cell density was 5×10⁶/ml and the inoculation dose was 0.1 ml/mice. The date of cell inoculation was defined as Day 0. When the average volume of the tumors in the control group reached about 100 mm³, mice were randomly assigned to different groups based on the tumor size. Injection was performed according to Table 3, and all the mice were euthanized on day 15;

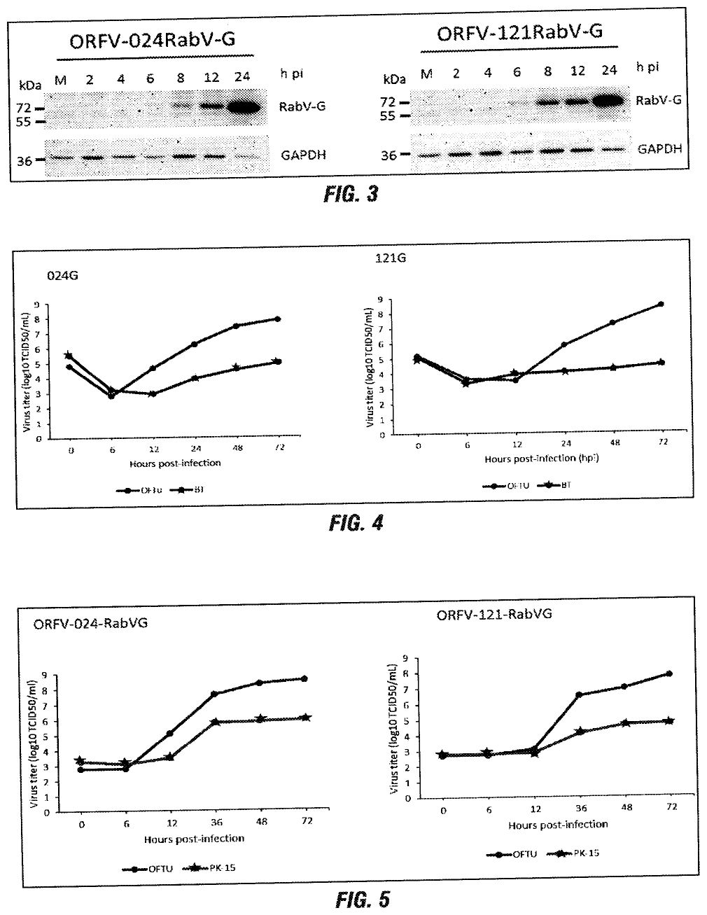

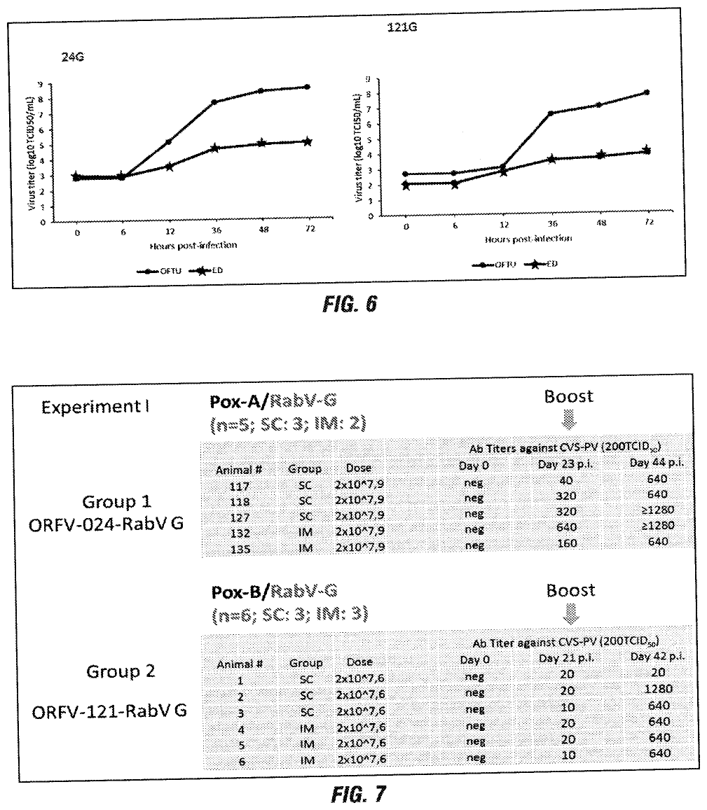

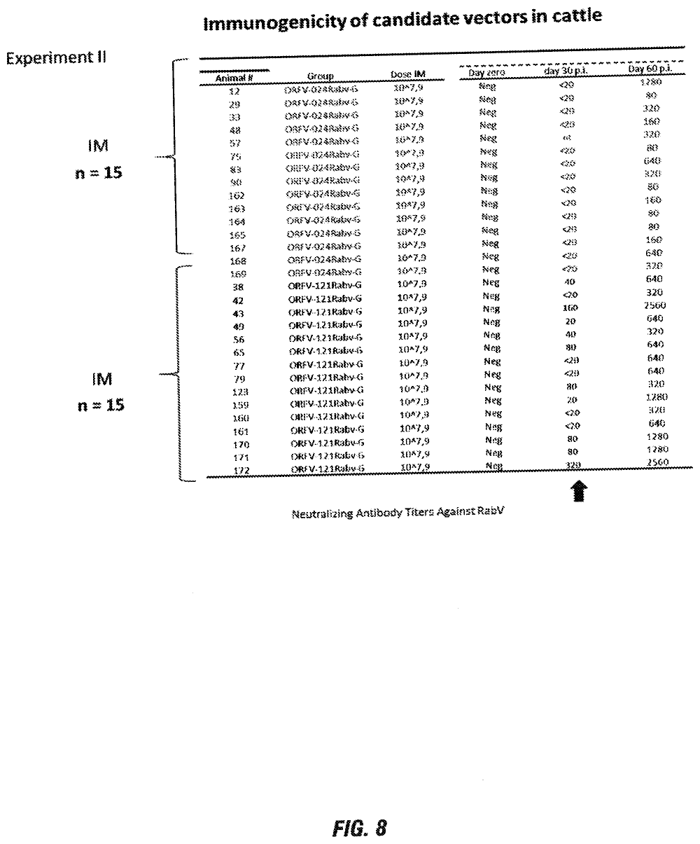

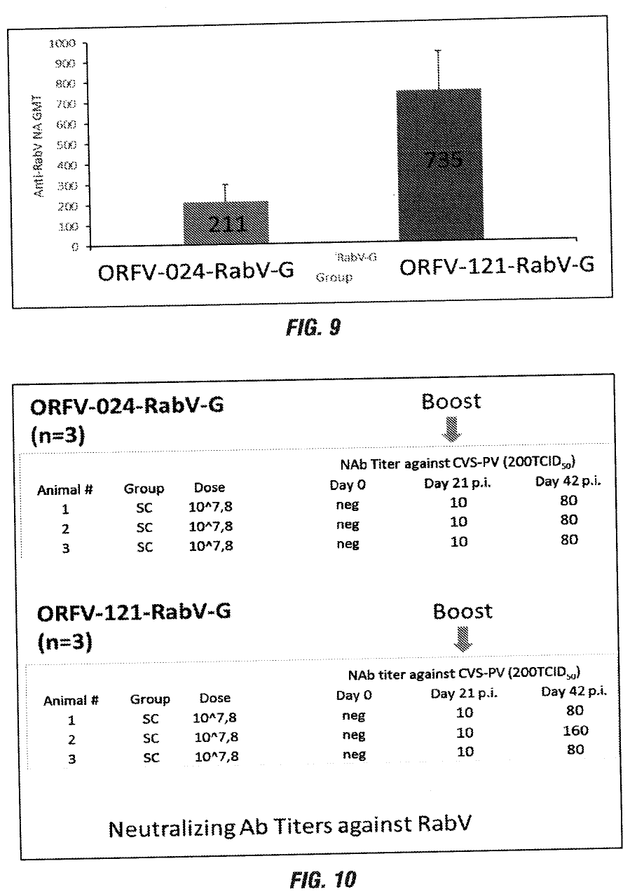

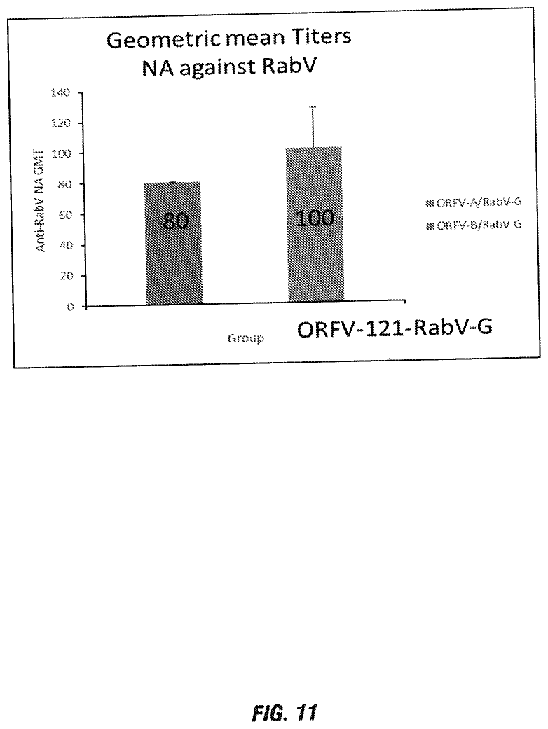

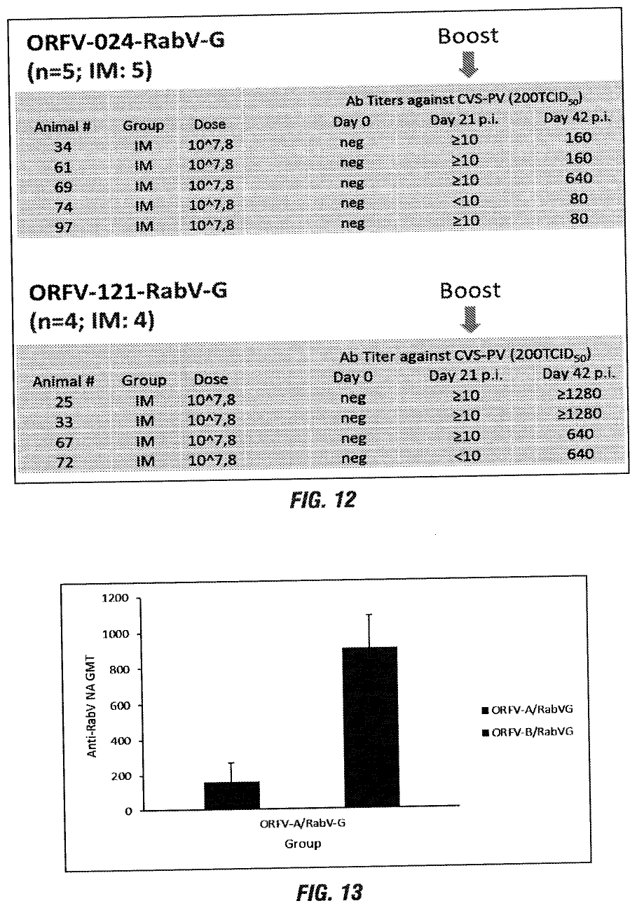

Weighed mice and measured tumor volumes twice a week throughout the entire study. The tumor volume was measured using a vernier caliper (Mahr GmbH, Model 16ER), and the results are shown in FIG. 3.

Calculated the tumor volume by the following formula: tumor volume (mm³)=a*b²/2, wherein a is the tumor length (mm), b is the tumor width (mm). Length and width were measured vertically.

Calculated the relative mean of tumor volumes by the following formula: RTV (Relative Tumor Volume)=Vₜ/V₀, wherein, V₀ is the average tumor volume measured during group assignment, and Vₜ is the average tumor volume at each measurement. Relative tumor proliferation rate T/C (%)=T_{RTV}/C_{RTV}*100%. (T_{RTV}: treatment group RTV; C_{RTV}: control group RTV).

Tumor growth inhibition rate TGI%=(1−T/C)×100%

ANOVA for statistical analysis: ANOVA analysis was used to compare whether there was a significant difference in the tumor volume between the experimental and control groups. All the data were analyzed with SPSS 17, and the statistical significance was defined as P<0.05.

D. The result

TABLE 4

| | Average | | TGI | |
|---|---|---|---|---|
| | Tumor | TGI | Analysis | P-value |
| Day 15 | Size | (%) | (TGI > 60%) | (ANOVA) |
| Control (PBS) | 3253.76 | — | — | — |
| POV-604-1D1 | 1524.13 | 53 | 2/5 | 0.023** |
| POV-601-3F8 | 2065.03 | 37 | 1/5 | 0.156 |
| POV-601-1A1 | 1357.58 | 58 | 3/5 | 0.012** |

### Example 7: Safety Evaluation of the ORFV Mutant

A. Experimental design

TABLE 5

| | Animal | Dosage and |
|---|---|---|
| Group | Number | Frequency |
| Control (PBS) | 3 | 400 μl/mouse s.c. |
| POV-601-1A1 | 3 | 400 μl/mouse s.c. |
| Control (PBS) | 3 | 0.1 ml/mouse i.v., |
| | | 2 doses within 24 h |
| POV-601-1A1 | 3 | 0.1 ml/mouse i.v., |
| | | 2 doses within 24 h |
| Control (PBS) | 2 | 300 μl/mouse s.c., |
| | | 3 doses, within 24 h, |
| | | one dose every 8 h |
| POV-601-1A1 | 2 | 300 μl/mouse s.c., |
| | | 3 doses, within 24 h, |
| | | one dose every 8 h |

s.c. = subcutaneous injection;
i.v. = intravenous injection (tail)

B. The testing substance and control

Testing substance: oncolytic virus ORFV POV-601-1A1; Control: PBS

C. Experimental animals: Female C57BL/6 mice, 6-8W, Zhejiang Vital River Laboratory Animal Technology Co., Ltd).

### D. Procedures

Female C57BL/6 mice were injected subcutaneously on their right back or through the tail vein with the testing substance or control. The date of the first injection was defined as Day 0. Mice were randomly assigned to groups and injection was performed according to Table 5. The experiment was completed on day 15. Mice were weighed daily for the first week and every two to three days thereafter for the duration of the study

Mice were weighed using the ML1602T electric balance (Mettler). The formula below was used to calculate the relative weight change:

Relative weight change (%)=[weight_{day_i}/weight_{day_0}]×100.

The results are shown in FIG. 4

### Example 8: Modulation of the Immune System by the ORFV Mutants

### A. Experimental Design

TABLE 6

| | | Route of | |
|---|---|---|---|
| | Animal | Adminis- | Dosage and |
| Group | Number | tration | Frequency |
| Control (PBS) | 3 | — | — |
| POV-601-1A1 | 3 | i.v. | 40 μl/mouse, a total of 4 doses |
| POV-601-1A1 | 3 | i.t. | 40 μl/mouse, a total of 4 doses |
| POV-601-1A1 | 3 | i.t. + i.v. | First i.t. 40 μl/mouse, |
| | | | followed by i.v., |
| | | | 0.1 ml/mouse |
| POV-601-1A1 | 3 | i.v. + i.t. | First i.v. 0.1 ml/mouse, |
| | | | followed by i.t., |
| | | | 1 h after i.v., |
| | | | 40 μl/mouse |

i.t. = intratumoral injection;
i.v. = intravenous injection (tail)

E. The testing substance and control:

Testing substance: oncolytic virus POV-601-1A1; Control: PBS

B. Experimental animals: female Balb/c-nude, 6W, Beijing Huafukang Biotechnology Co., Ltd

### C. Procedure

Implanted female Balb/c-nude mice subcutaneously on their bilateral back with C-33A cells (Cell Bank of Type Culture Collection Committee of Chinese Academy of Sciences) resuspended in PBS; the cell density was 1×10⁸/ml and the implantation dose was 0.1 ml/mice. The date of cell implantation was defined as Day 0. When the average volume of the tumors reached about 100 mm³, injection was performed according to Table 6. The experiment was completed 24 h after the last injection. Upon completion of the experiment, blood and tumors were collected from mice. Tumors were cut, digested and resuspended in PBS into a single cell suspension. Cells were then labeled with fluorescent antibodies (Anti-mouse CD45 APC-eFluor 780, Anti-mouse CD49b-PE, Anti-mouse CD69 APC), and analyzed by a flow cytometer (Thermo, Model AFC2), see FIG. 5 (sub panels A-C).

### Example 9 In Vitro Cell Infection Effects of the ORFV Mutant

Viral strain: POV-601-1A1
1) Trypsinized the cultured tumor cells and counted them. Cells were seeded into 96-well plates at 1.5×10⁴cell/well and were cultured for 24 hours before exposing to the virus;
2) Diluted the viral solution with a 2% FBS complete medium to 4 different concentrations: 1.5×10⁸ pfu/ml, 1.5×10⁷ pfu/ml, 1.5×10⁶ pfu/ml, 1.5×10⁵ pfu/ml;
3) Added the diluted viral solutions into the 96-well cell plates, 100 ul/well, and the final MOI of the added virus for each well is of 1000, 100, 10, 1, respectively, and three duplicates were set for each MOI;
4) Set three wells and added the 2% FBS complete medium as the negative control;
5) Rocked gently to let the viral solution cover the surface of the monolayer cell evenly, and ensured that every conners were covered;
6) Transferred the plates seeded with the control or the viral solution back into a 37° C., 5% CO2 incubator, and cultured cells for 72 h;
7) Took out the 96-well plates from the incubator, and added 10 ul of Alamar Blue Cell Viability Reagent (Invitrogen, Cat#: 2072060) into each well, rocked the plates gently to let the reagent (dye) disperse evenly. Covered the plates with aluminum foils, transferred them back into the 37° C. incubator, and incubated without light;
8) After the incubation was complete, took off the lids, and put the 96-well plates into a microplate reader (Molecular Devices, model SPECTRAMAX M4). Set the excitation wavelength at 560 nm and the emission wavelength at 590 nm, carried out the absorbance detection, calculated the cell death ratio using the following formula, and analyzed the result:

$$( 1 - \frac{Absorbance of a particular MOI}{Absorbance of MOI 0} ) \times 1 \; 00 \% ;$$

9) The result has shown that the POV-601-1A1 virus had an infectivity to melanoma cancer cell (B16-F10), bladder cancer cell (MB49), liver cancer cell (Hepa1-6), colon cancer cell (CT26), human cervical cancer cell (C-33A), human ovarian adenocarcinoma (SK-OV-3), and etc, and viral infection was increased with a increase in MOI. However, it barely infected Vero cells.

TABLE 7
The Result of In Vitro Infection of the POV-601-1A1 Virus to Tumor Cells

| | | | | | | | | Vero |
|---|---|---|---|---|---|---|---|---|
| | | MB49 | C-33A | Hepa1-6 | CT26.WT | SK-OV-3 | LLC | African |
| | B16-F10 | Mouse | Human | Mouse | Mouse | Human | Mouse | Green |
| | Melanoma | Bladder | Cervical | Liver | Colon | Ovarian | Lung | Monkey |
| | Cancer | Cancer | Cancer | Cancer | Cancer | Cancer | Cancer | Kidney |
| MOI | Cell | Cell | Cell | Cell | Cell | Cell | Cell | Cell |
| 0 | 0.00% | 0.00% | 0.00% | 0.00% | 0.00% | 0.00% | 0.00% | 0.00% |
| 1 | 12.39% | 4.49% | −6.06% | 5.84% | 9.71% | 2.11% | 2.46% | 11.13% |
| 10 | 14.16% | 29.77% | 25.05% | 25.55% | 14.51% | 25.06% | 7.10% | 12.48% |
| 100 | 40.14% | 74.78% | 78.49% | 69.20% | 65.32% | 58.22% | 81.70% | 11.51% |
| 1000 | 85.60% | 82.94% | 88.88% | 75.29% | 85.53% | 65.16% | 95.04% | −36.81% |

### Example 8: Evaluation of Antitumor Activity of the ORFV Mutants in A Mouse Colon Cancer Model

A. Experimental design

TABLE 8

| | | | Route of | |
|---|---|---|---|---|
| | Animal | Virus | Adminis- | Dosage and |
| Group | Number | Titer | tration | Frequency |
| Control (PBS) | 6 | / | Intratumoral | First injection |
| POV-601-3F8 | 6 | 2.5*10⁸ | injection | upon the day of |
| | | pfu/ml | (i.t) | grouping, |
| POV-601-1A1 | 6 | 2.5*10⁸ | | followed by one |
| | | pfu/ml | | injection every |
| | | | | 72 h, 40 μL/mouse |

B. Experimental animals: Female Balb/c mice, 6-8W, Zhejiang Vital River Laboratory Animal Technology Co., Ltd).

### C. Procedures

Implanted female Balb/c mice subcutaneously on their right back with CT-26 cells (Cell Bank of Type Culture Collection Committee of Chinese Academy of Sciences) resuspended in PBS; the cell density was 5×10⁶/ml and the implantation dose was 0.1 ml/mice. The date of cell implantation was defined as Day 0. When the average volume of the tumors in the control group reached about 100 mm³, mice were randomly assigned to different groups based on the tumor size. Injection was performed according to Table 8, and all the mice were euthanized on day 25;

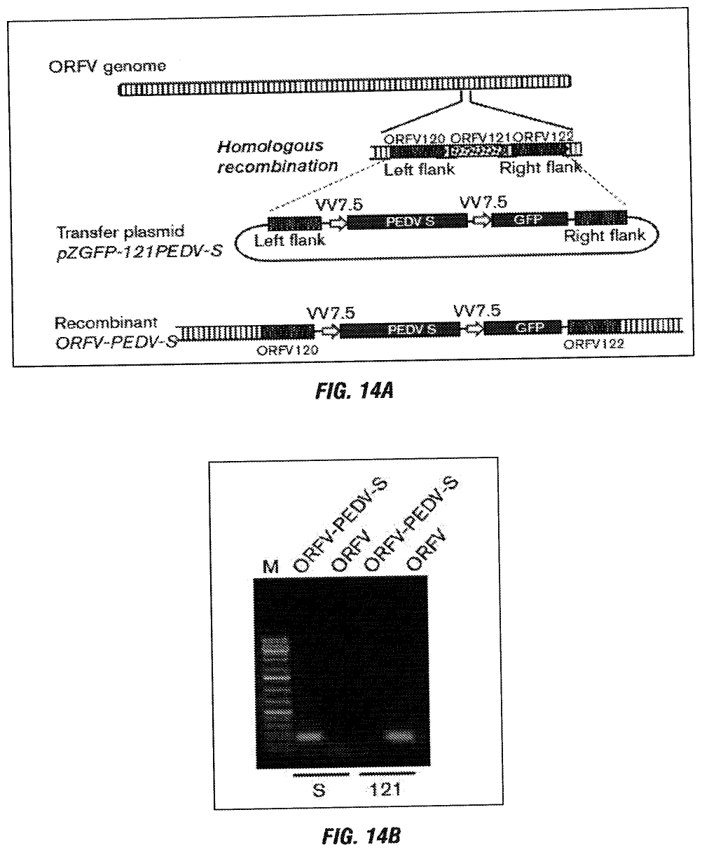

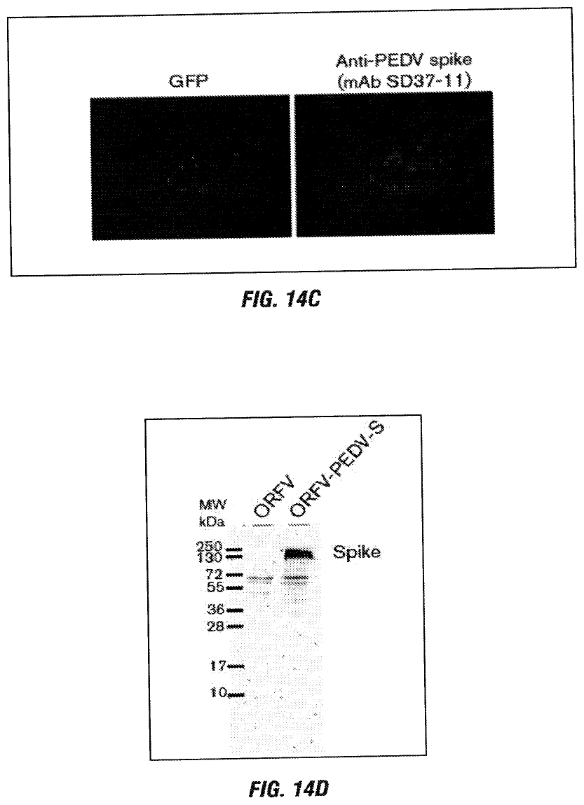

Weighed mice and measured the tumor volumes twice a week throughout the entire study. The tumor volume was measured using a vernier caliper (Mahr GmbH, Model 16ER), and the results are shown in FIG. 14.

Calculated the tumor volume by the following formula: tumor volume (mm³)=a*b²/2, wherein a is the tumor length (mm), b is the tumor width (mm). Length and width were measured vertically.

Calculated the relative mean of tumor volumes by the following formula: RTV (Relative Tumor Volume)=Vₜ/V₀. Wherein, V₀ is the average tumor volume measured during group assignment, and Vₜ is the average tumor volume at each measurement. Relative tumor proliferation rate T/C (%)=T_{RTV}/C_{RTV}*100%. (T_{RTV}: treatment group RTV; C_{RTV}: control group RTV).

Tumor growth inhibition rate TGI%=(1−T/C)×100%

ANOVA for statistical analysis: ANOVA analysis was used to compare whether there is a significant difference in tumor volumes between the experimental and control groups. All the data were analyzed with SPSS 17, and the statistical significance was defined as P<0.05.

### D. Result

TABLE 9

| | Average | | TGI | |
|---|---|---|---|---|
| | tumor | TGI | analysis | P-value |
| Day 25 | size | (%) | (TGI > 60%) | (ANOVA) |
| Control (PBS) | 3587.22 | — | — | — |
| POV-601-3F8 | 2136.75 | 39 | 1/6 | 0.075 |
| POV-601-1A1 | 1757.35 | 51 | 4/6 | 0.028* |

### Example 9 Evaluation of Antitumor Activity of The ORFV Mutants in a Mouse Melanoma Model

Constructed the ORFV mutants with complete or partial deletions of the coding and/or non-coding regions of the ORFV111 and/or ORFV112 genes with recombinant methods and the (deletion) effects were tested using the methods recorded (described) in Example 6, unless otherwise specified.

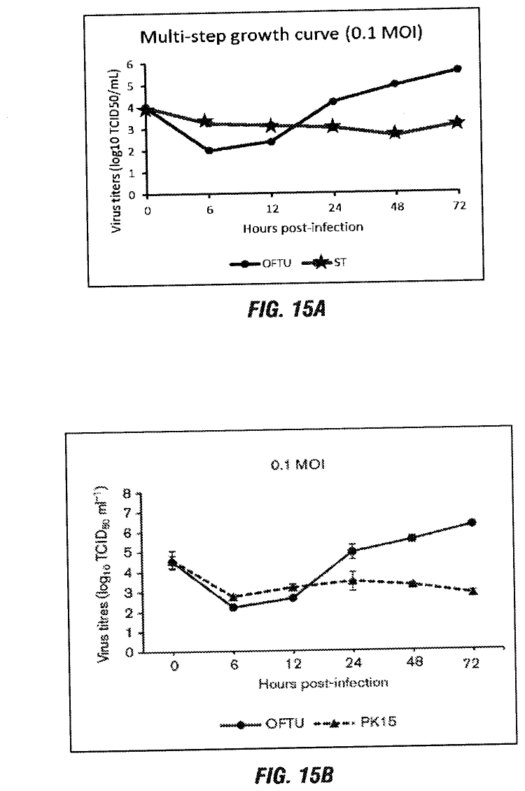

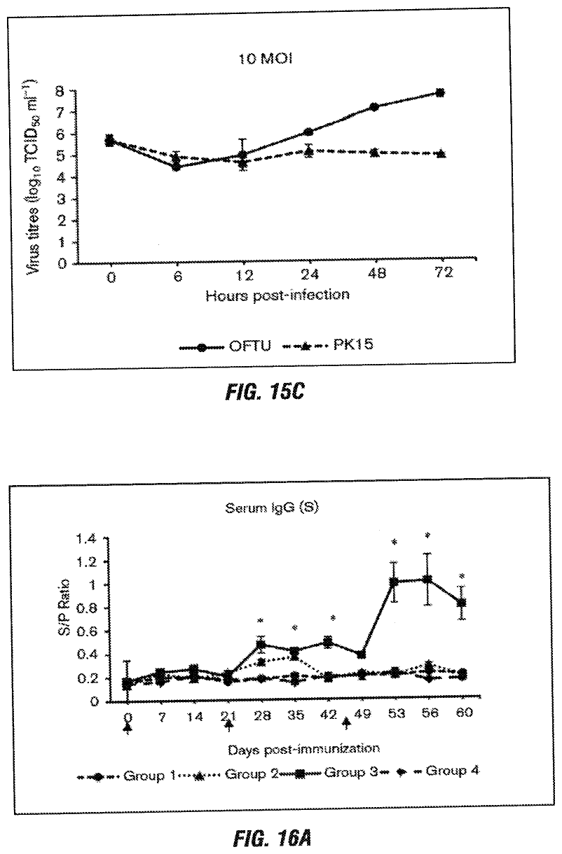

TABLE 10
(see FIG. 15)

| | | Average | | TGI | |
|---|---|---|---|---|---|
| | Animal | Tumor | TGI | Analysis | P-value |
| Day 18 | Number | Size | (%) | (TGI > 60%) | (ANOVA) |
| Control (PBS) | 6 | 6420 | — | — | — |
| v611a | 6 | 2306 | 64 | 3/6 | 0.002** |
| (2.0*10⁸ pfu/ml) | | | | | |
| POV-601-1A1 | 6 | 2812 | 56 | 1/6 | 0.007** |
| (v601-1A1) | | | | | |
| (2.0*10⁸ pfu/ml) | | | | | |

v611a: the mutant with a complete deletion of the ORFV112 gene

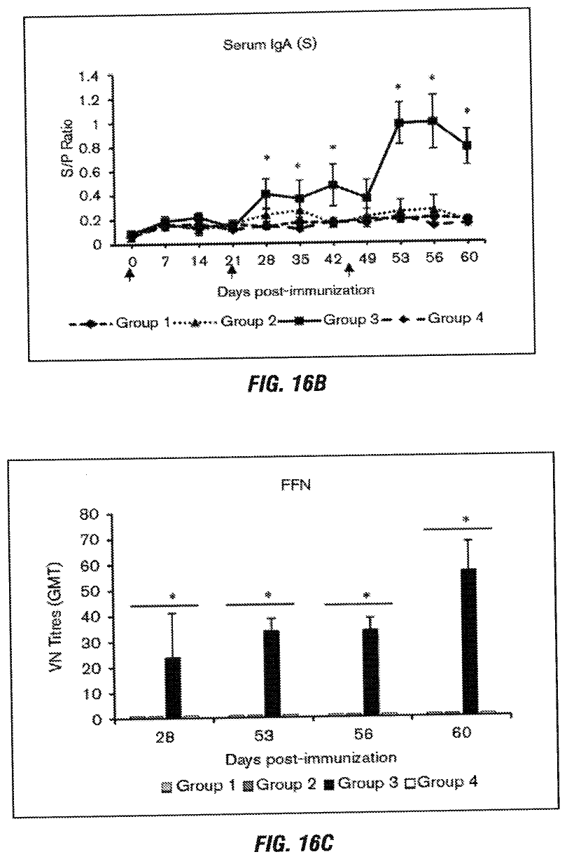

TABLE 11
(see FIG. 16)

| Cell density | | Average | | TGI | |
|---|---|---|---|---|---|
| 3 × 10⁶/ml | Animal | size | TGI | Analysis | P-value |
| Day 19 | Number | tumor | (%) | (TGI > 60%) | (ANOVA) |
| Control (PBS) | 6 | 3983.35 | — | — | — |
| v615a | 6 | 1688.99 | 58 | 2/6 | 0.002** |
| (1.37*10⁸ pfu/ml) | | | | | |
| v616a | 6 | 1747.31 | 56 | 3/6 | 0.002** |
| (2.0*10⁸ pfu/ml) | | | | | |
| POV-601-1A1 | 6 | 1576.46 | 60 | 4/6 | 0.001** |
| (v601-1A1) | | | | | |
| (2.0*10⁸ pfu/ml) | | | | | |

v615a: the mutant with a complete deletion of the coding regions of the ORFV111 and ORFV112 genes
v616a: the mutant with a complete deletion of the ORFV111 and ORFV112 genes

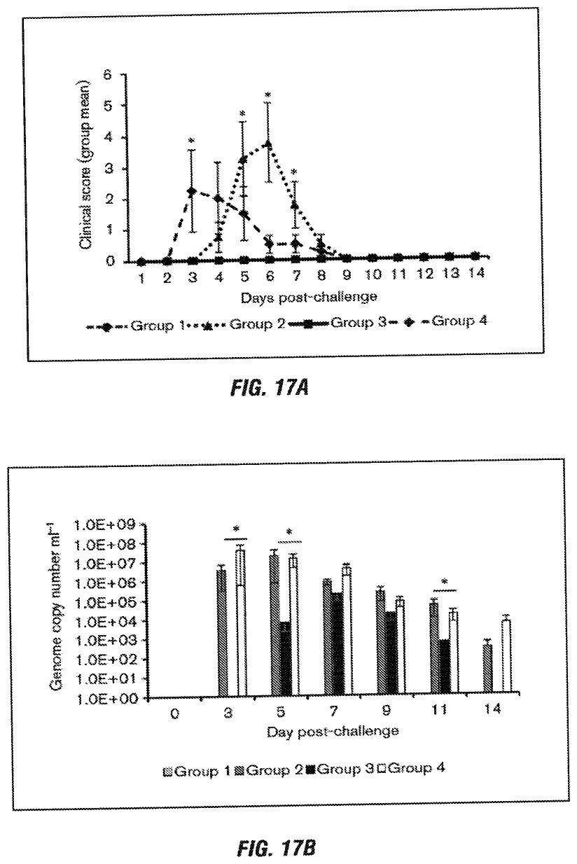

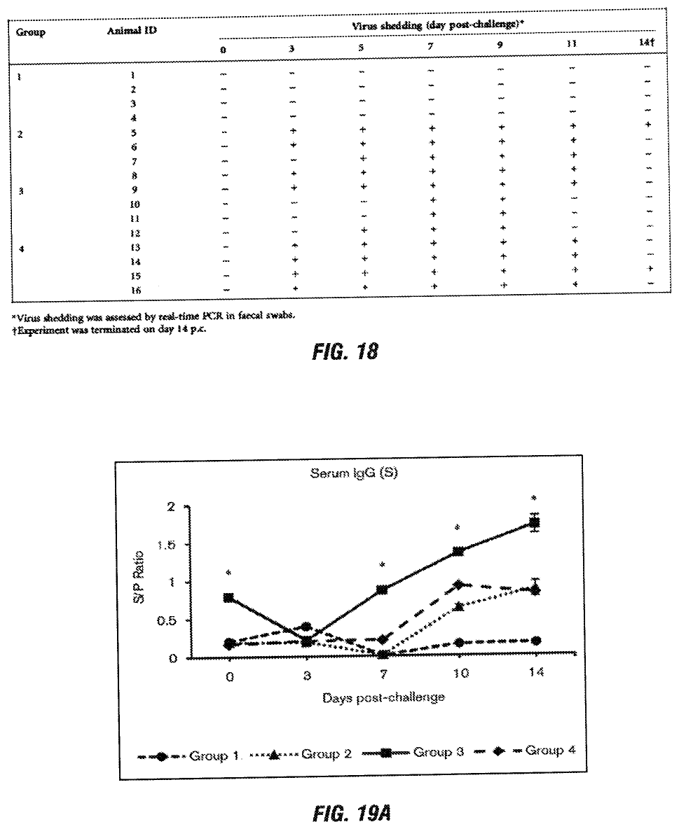

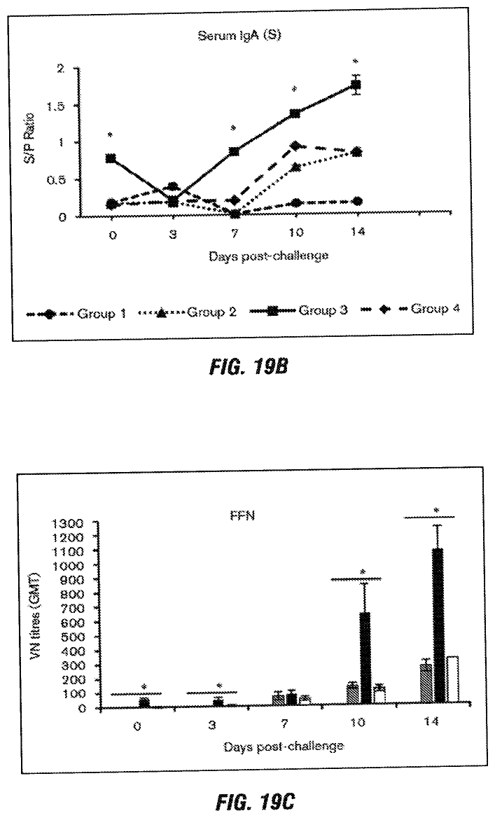

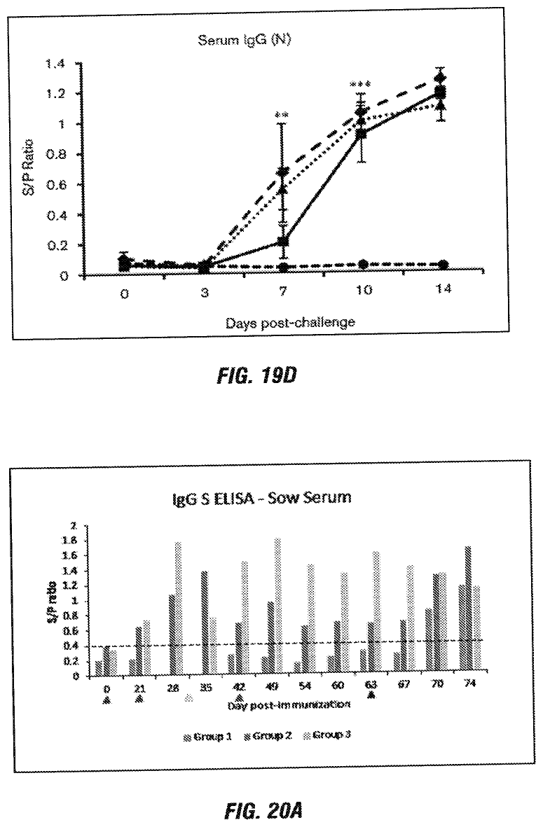

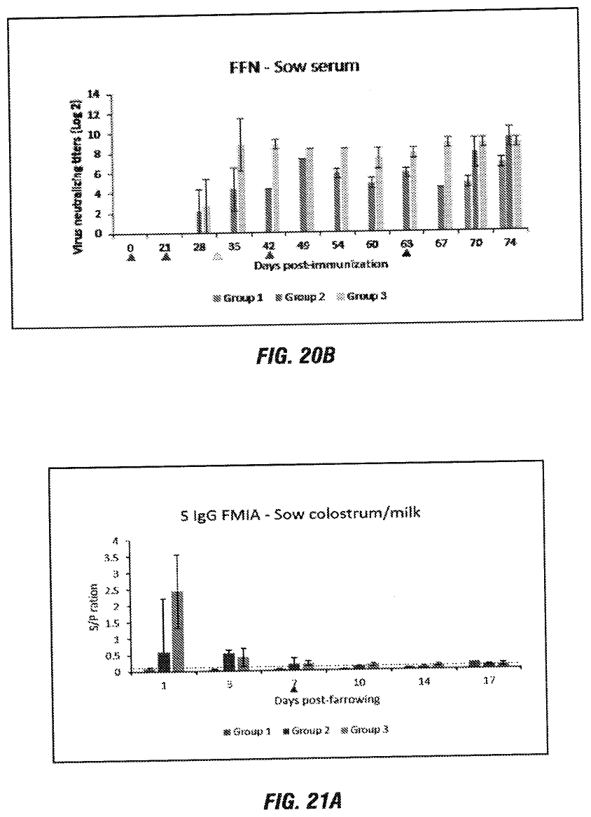

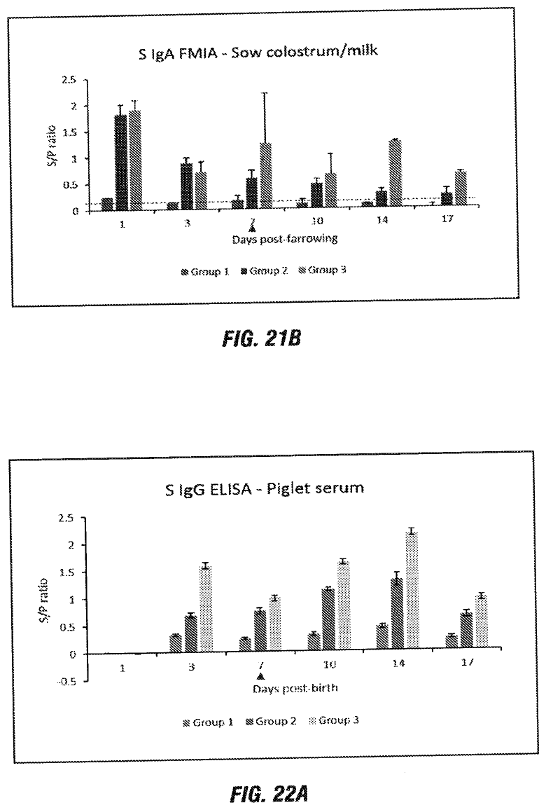

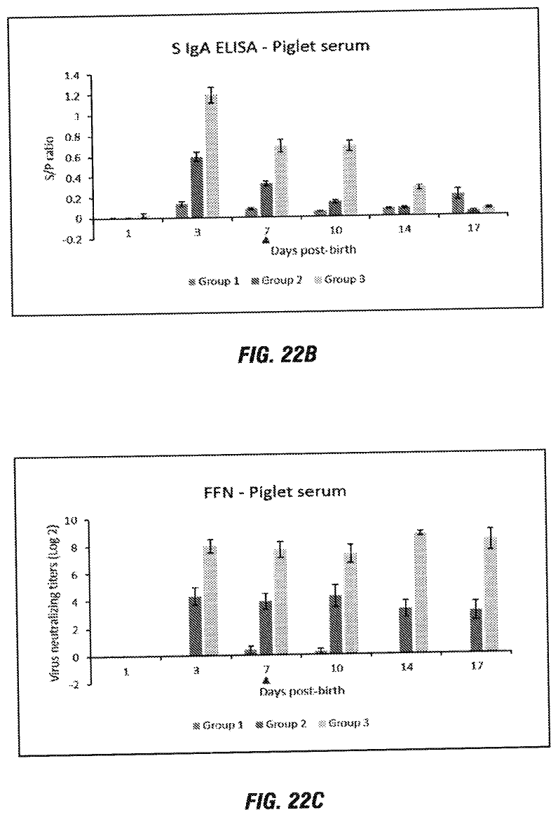

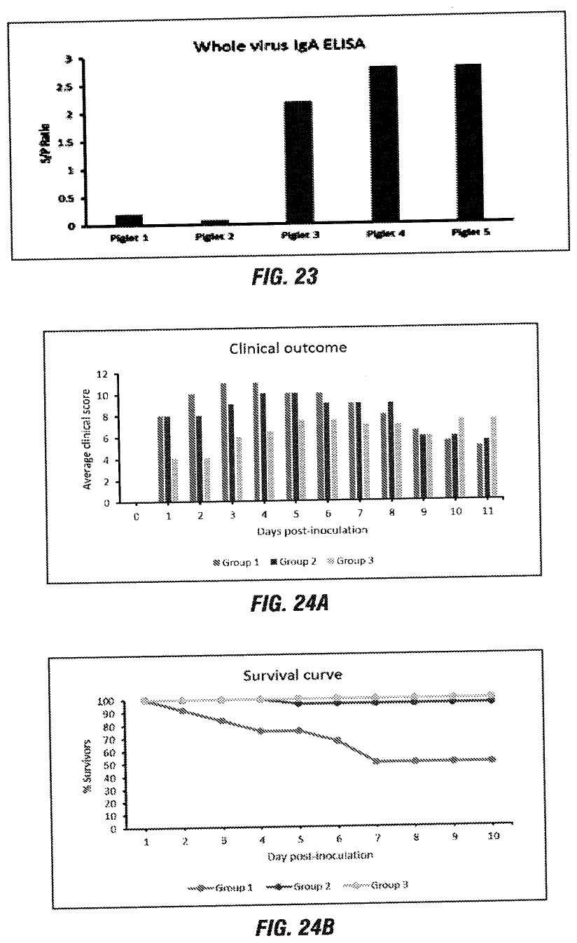

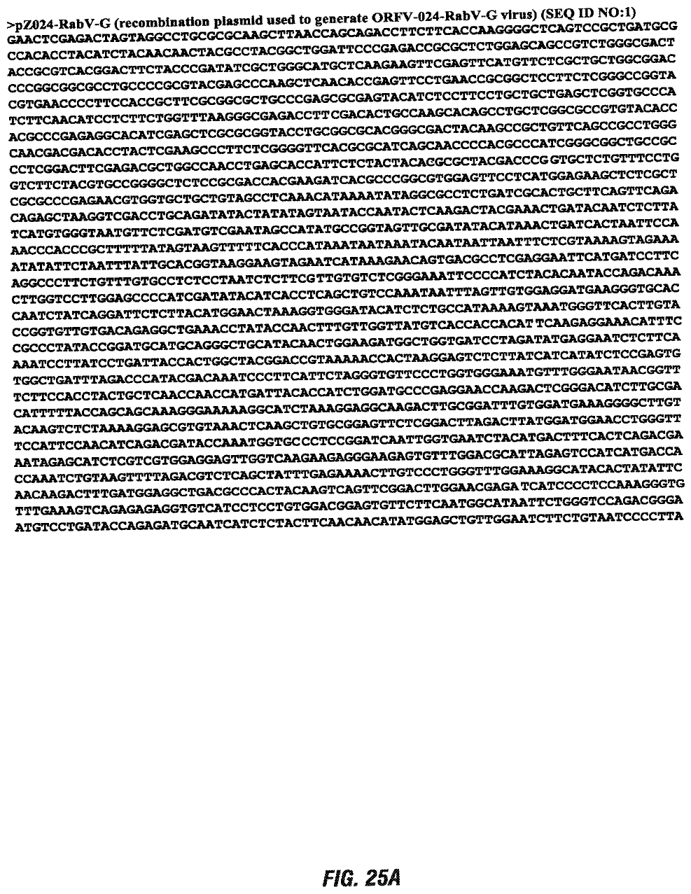

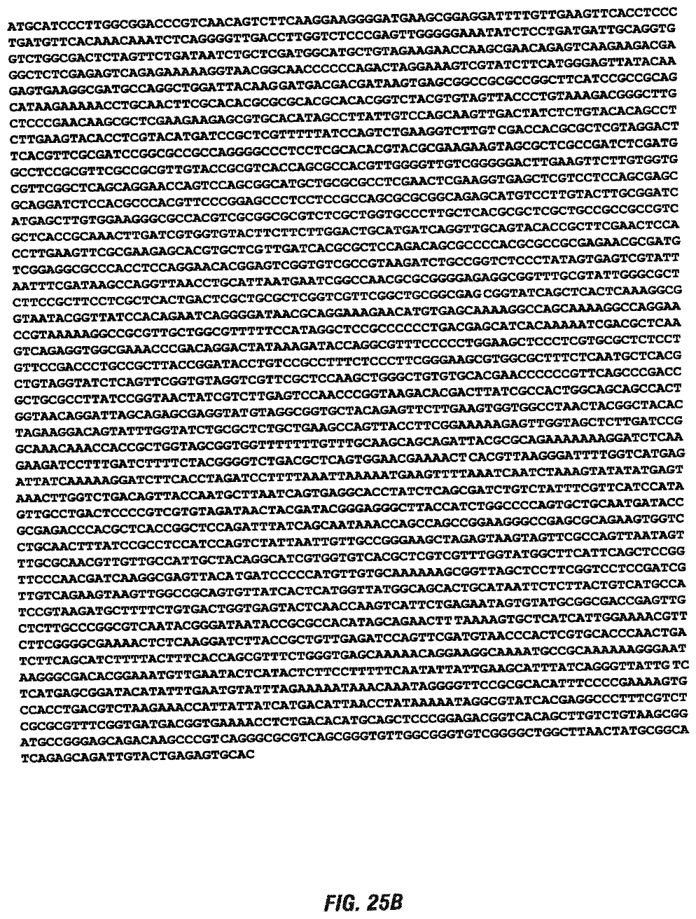

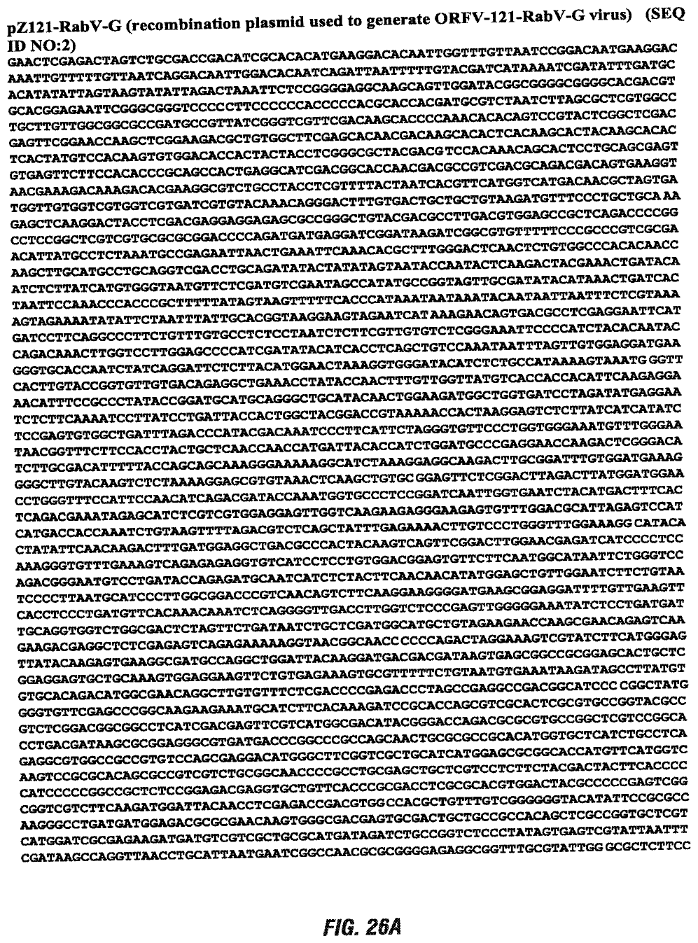

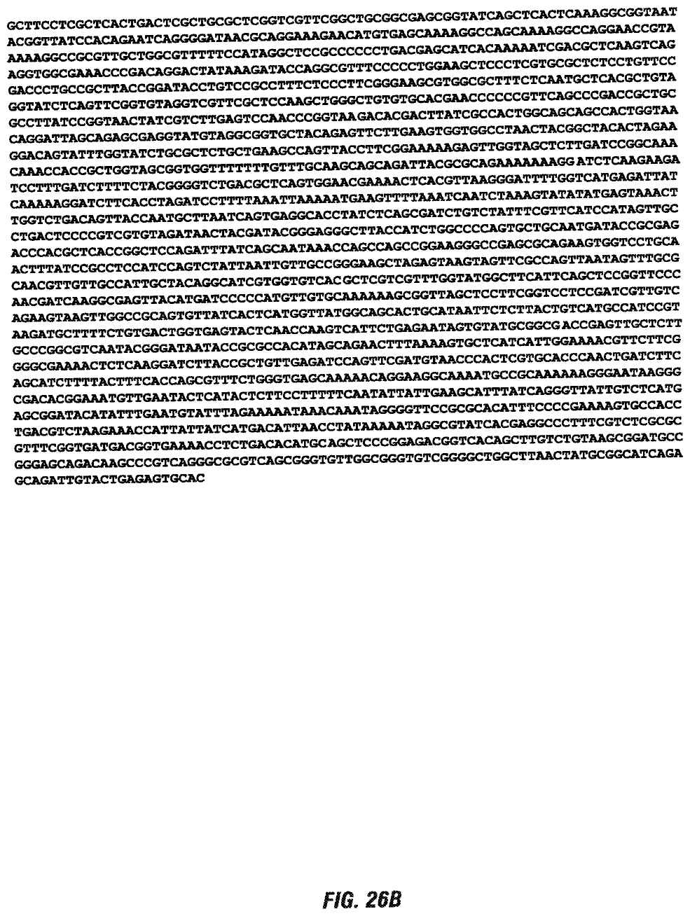

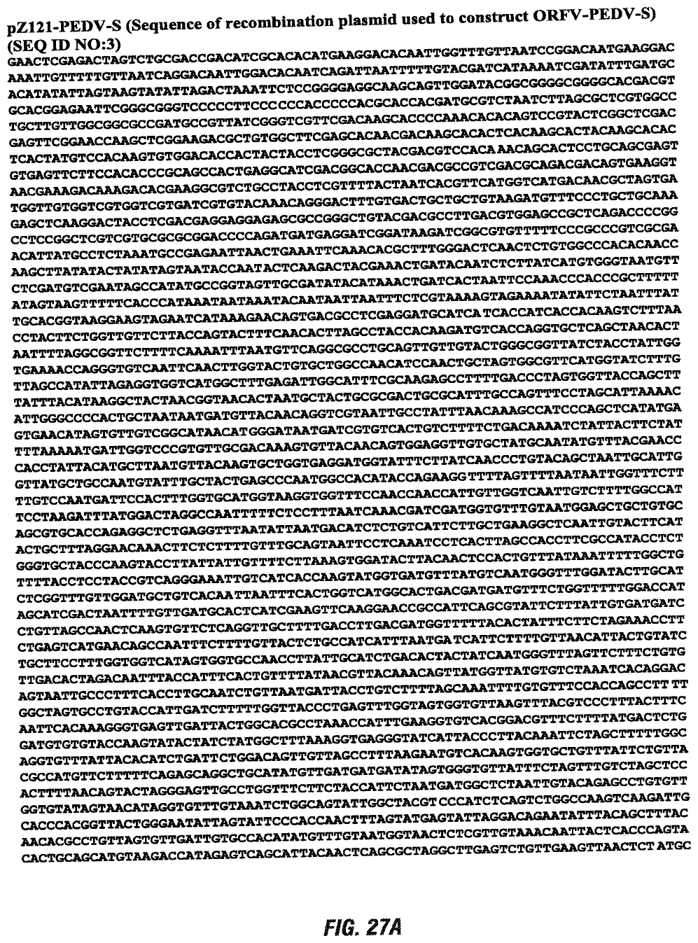

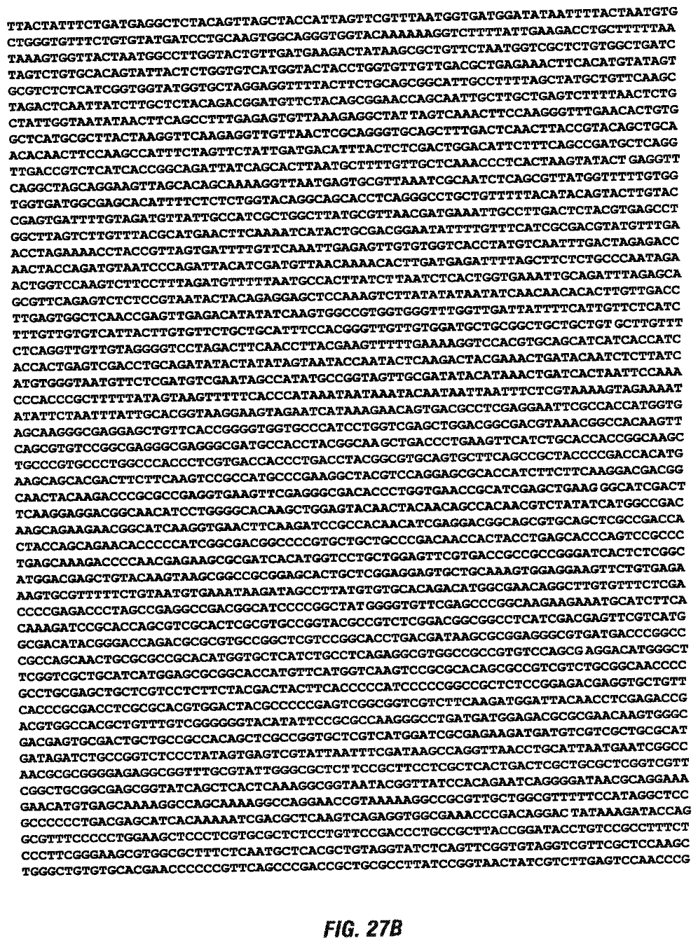

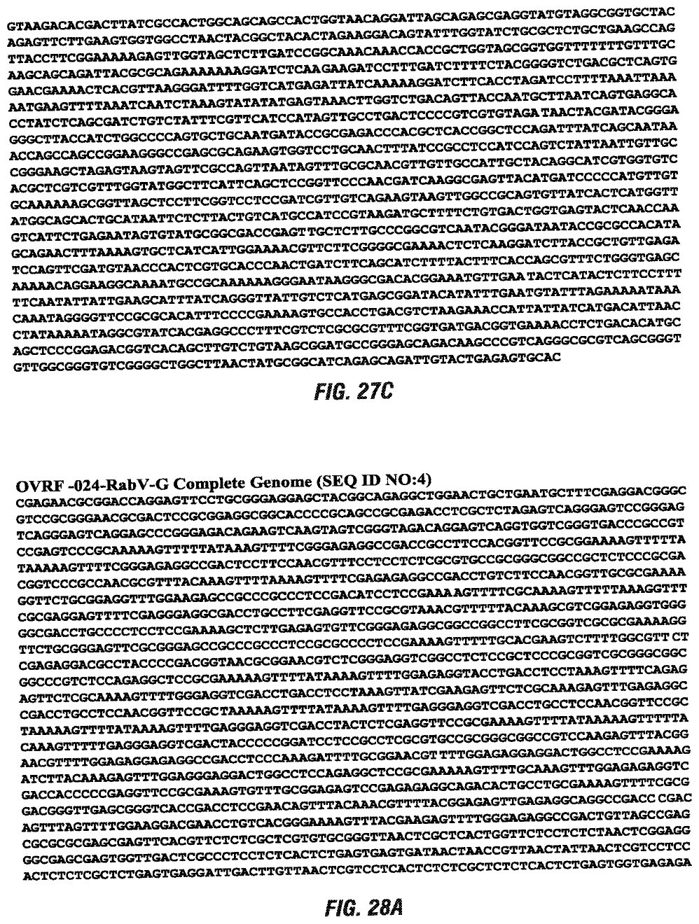

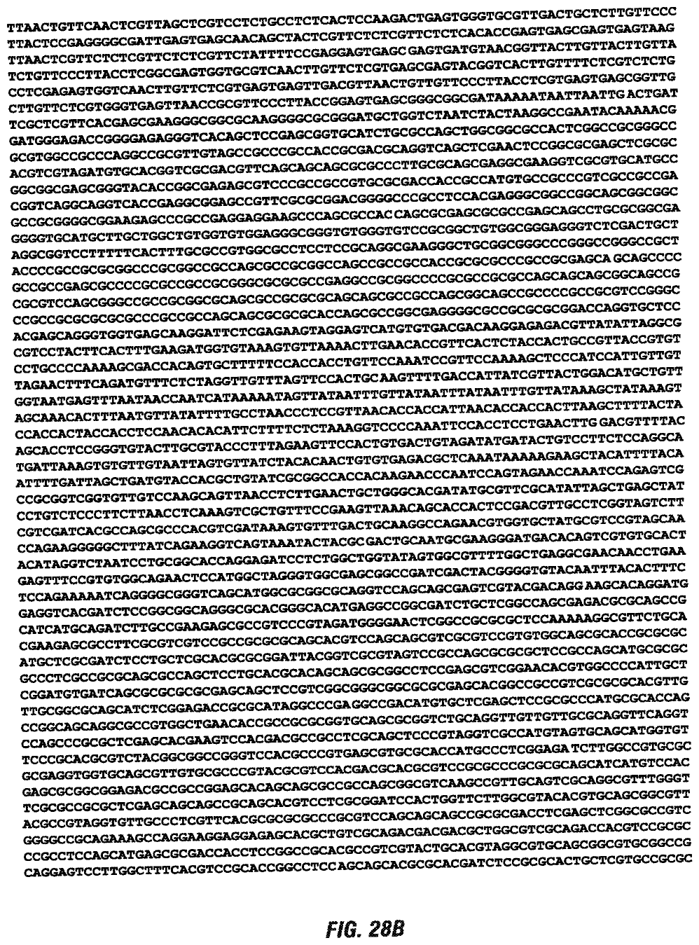

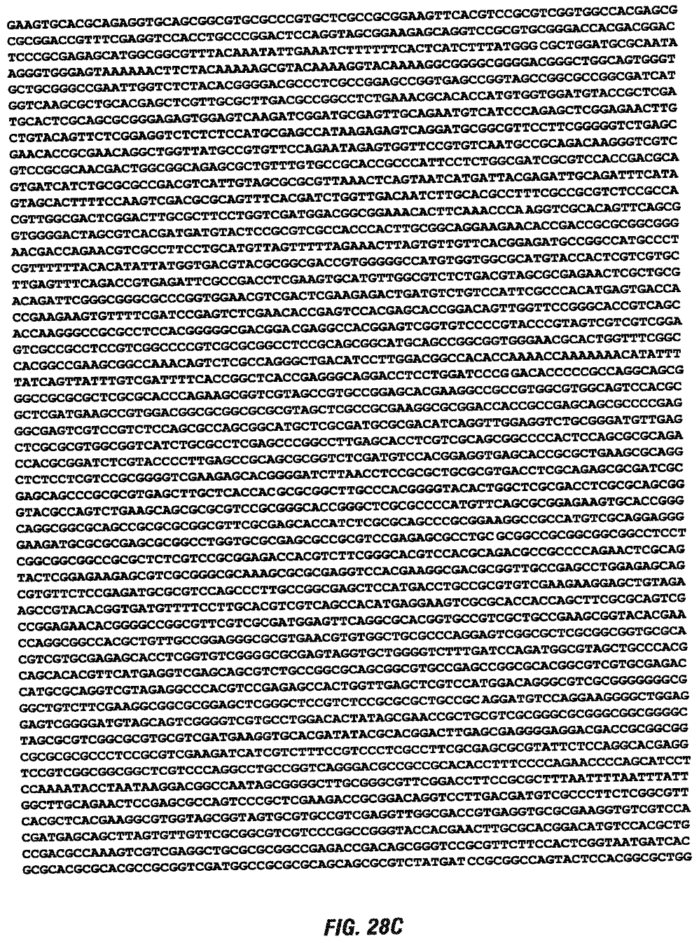

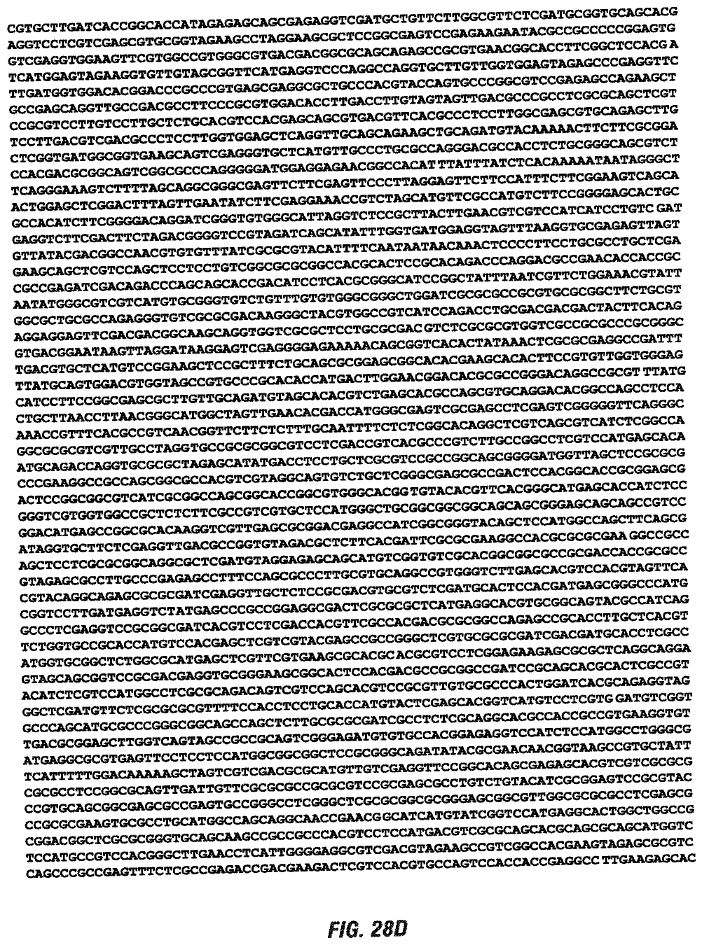

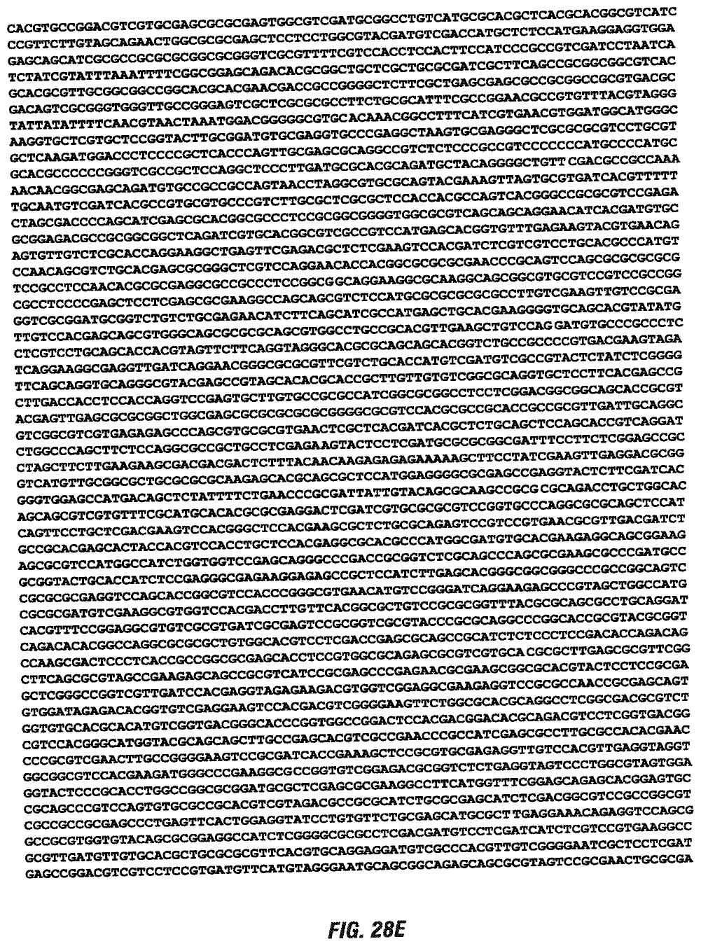

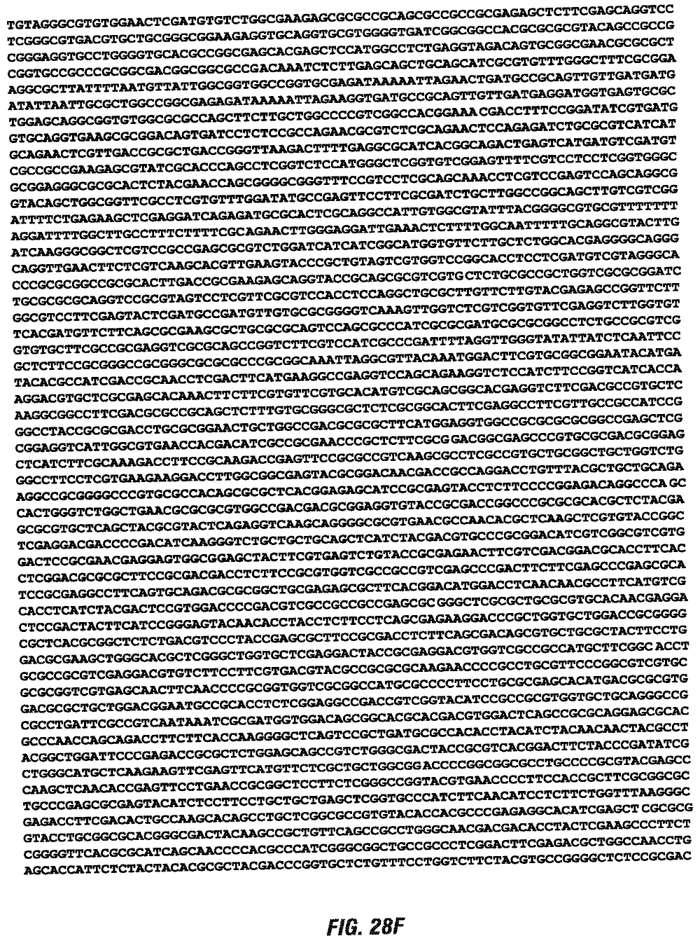

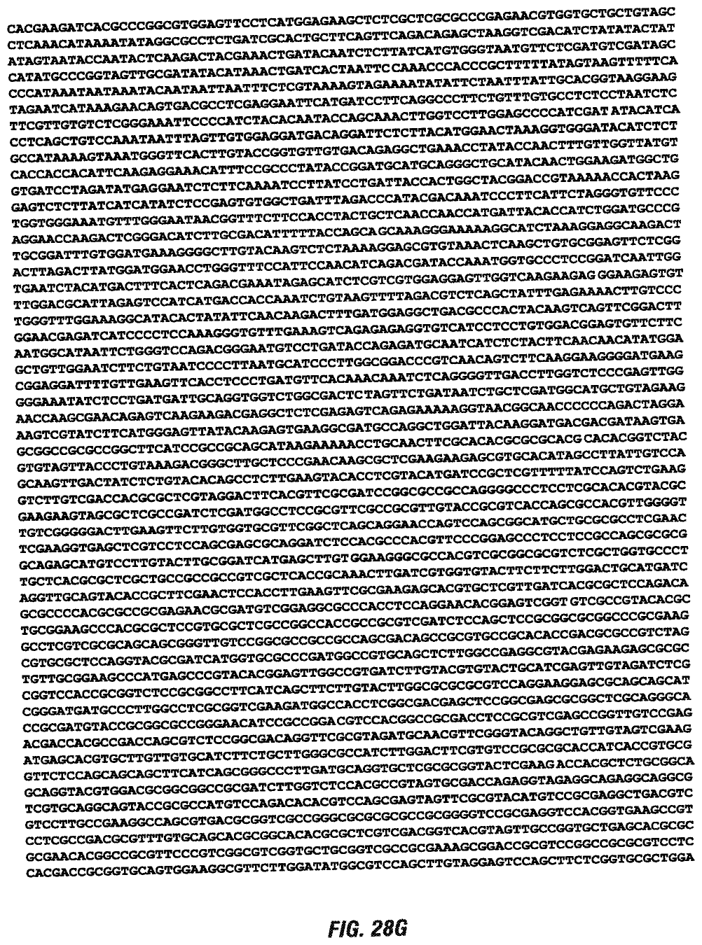

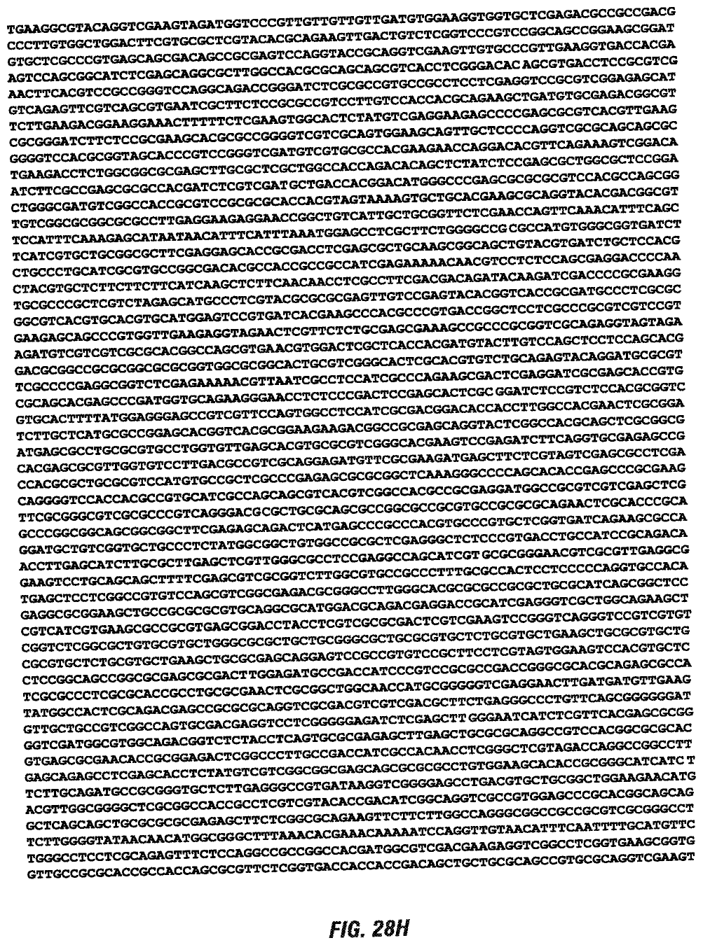

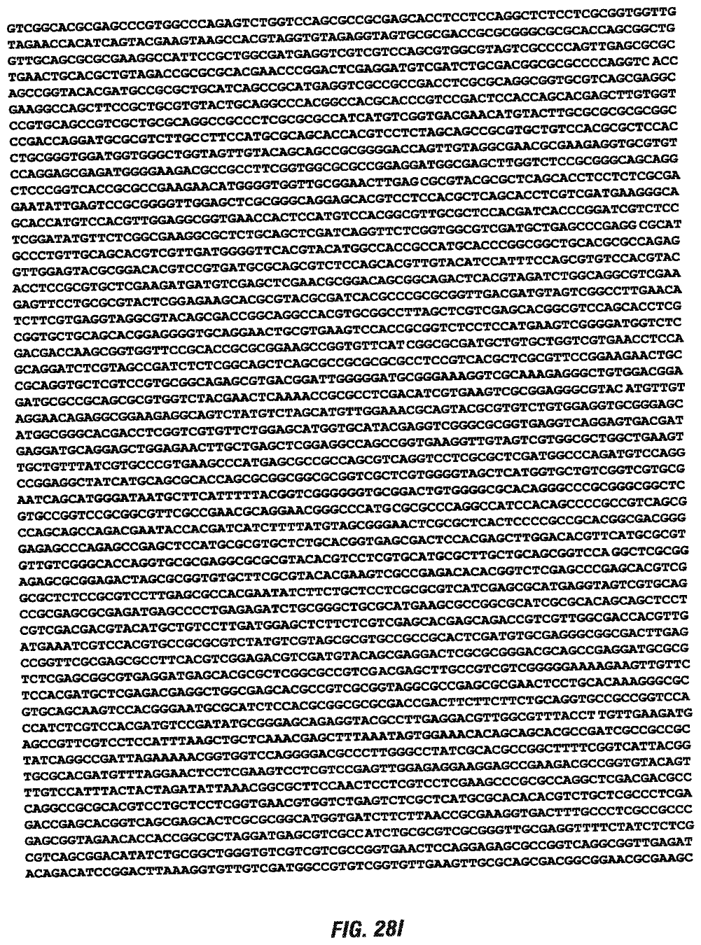

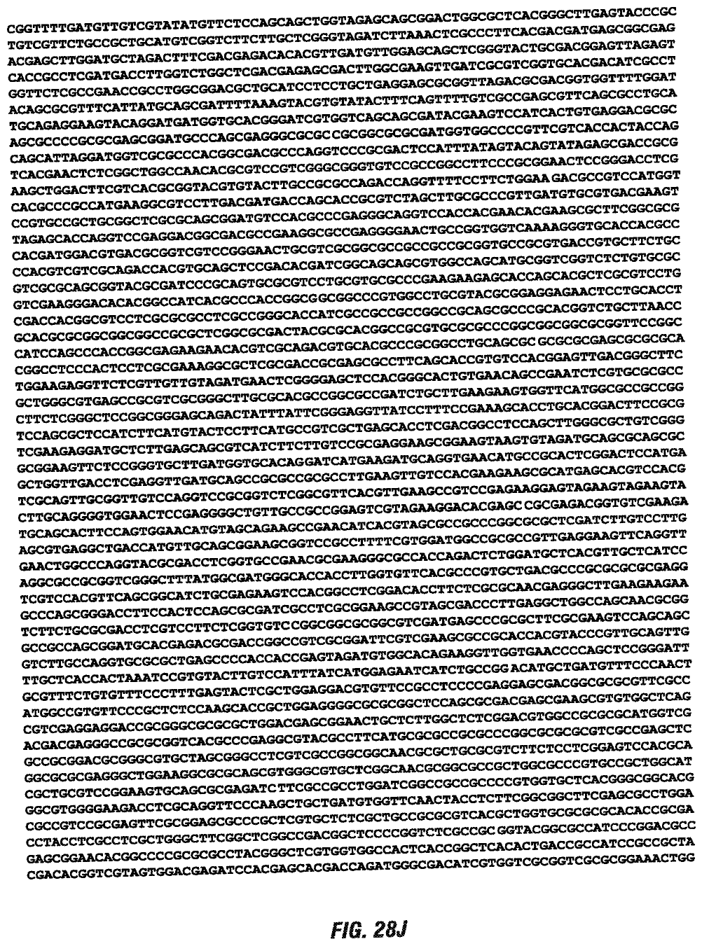

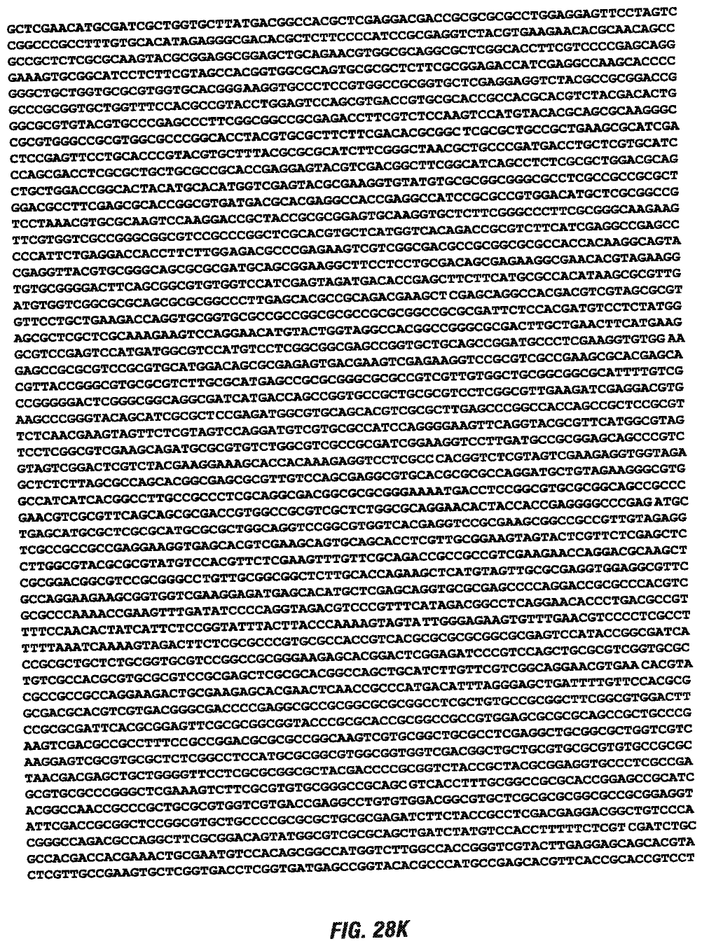

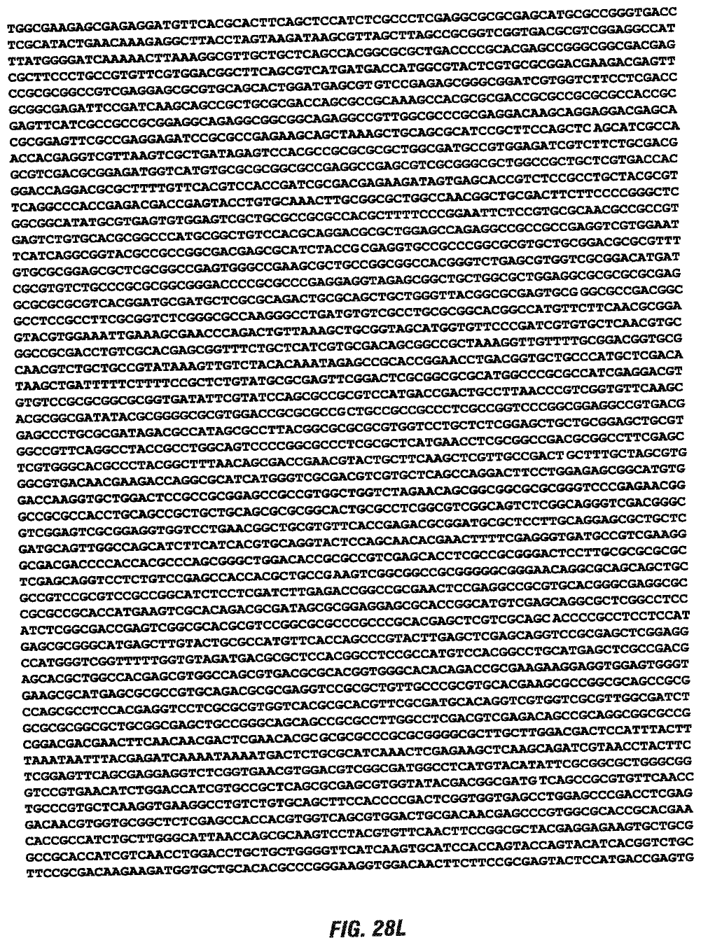

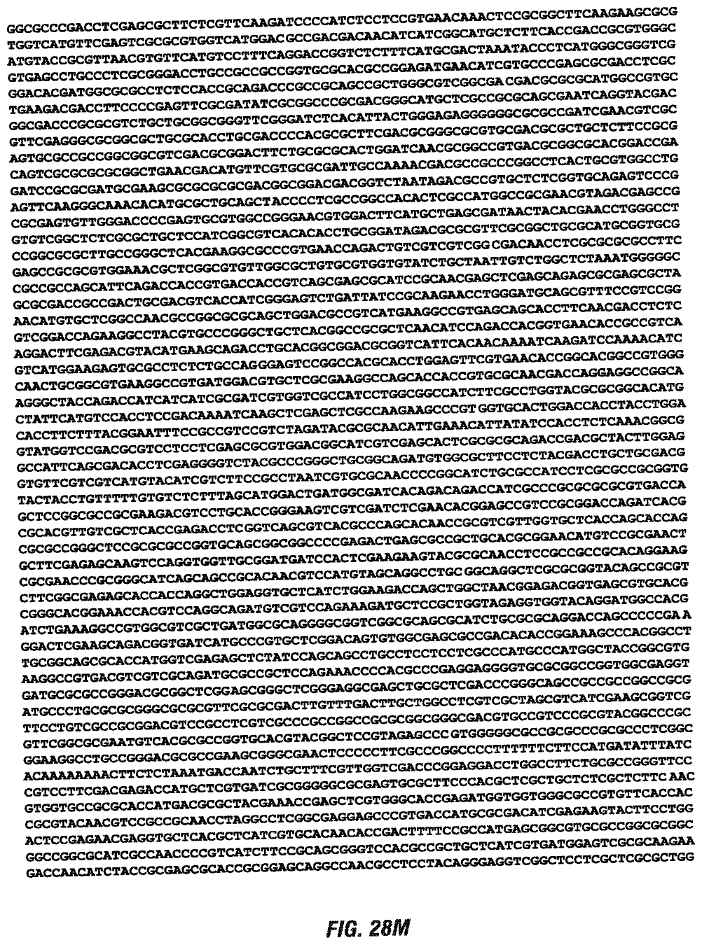

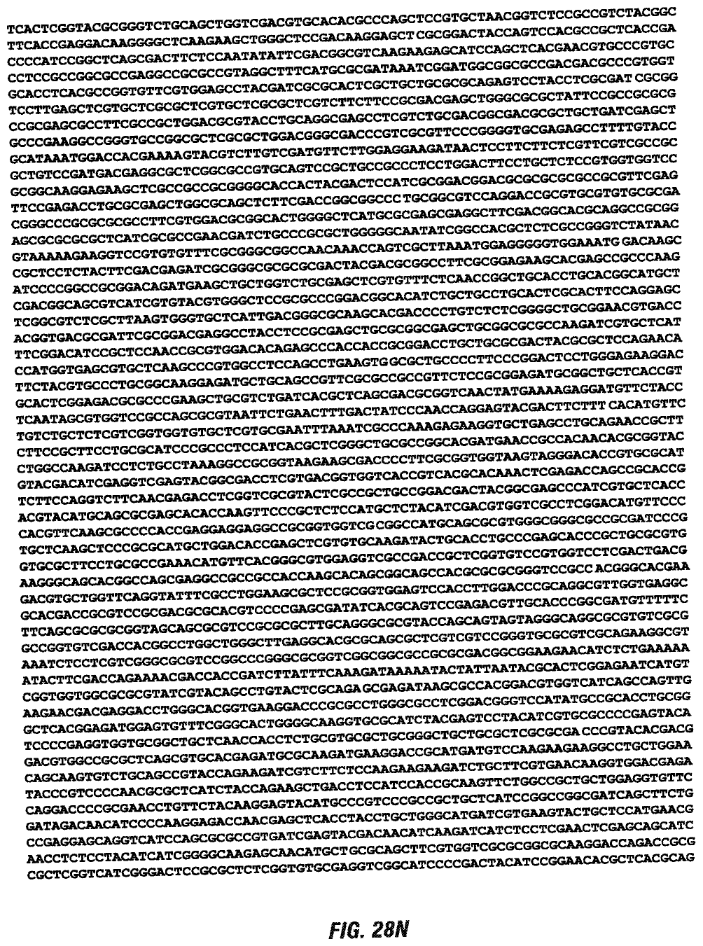

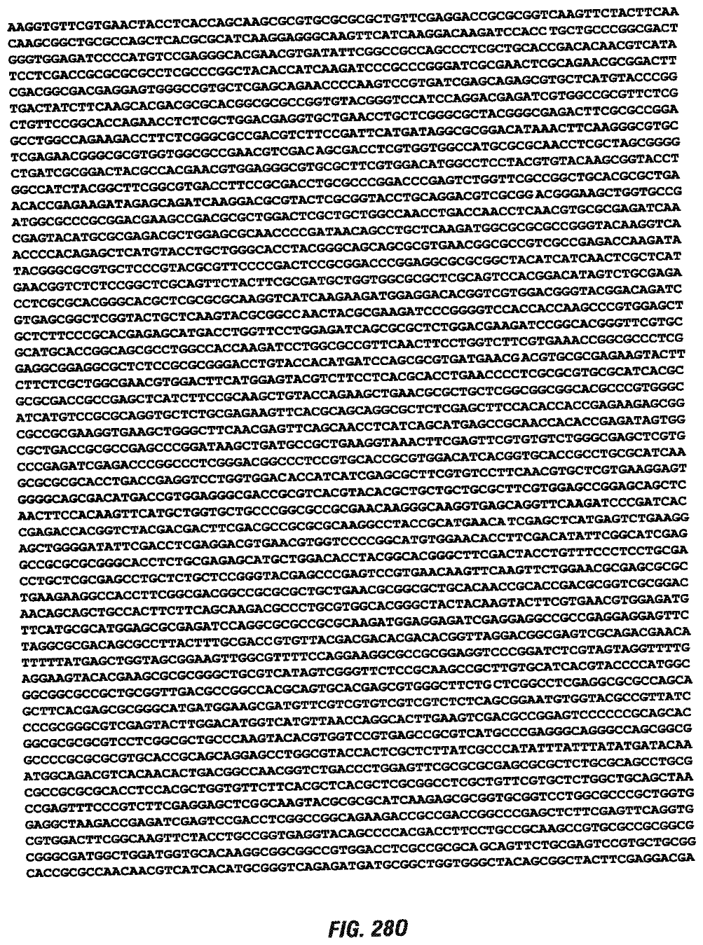

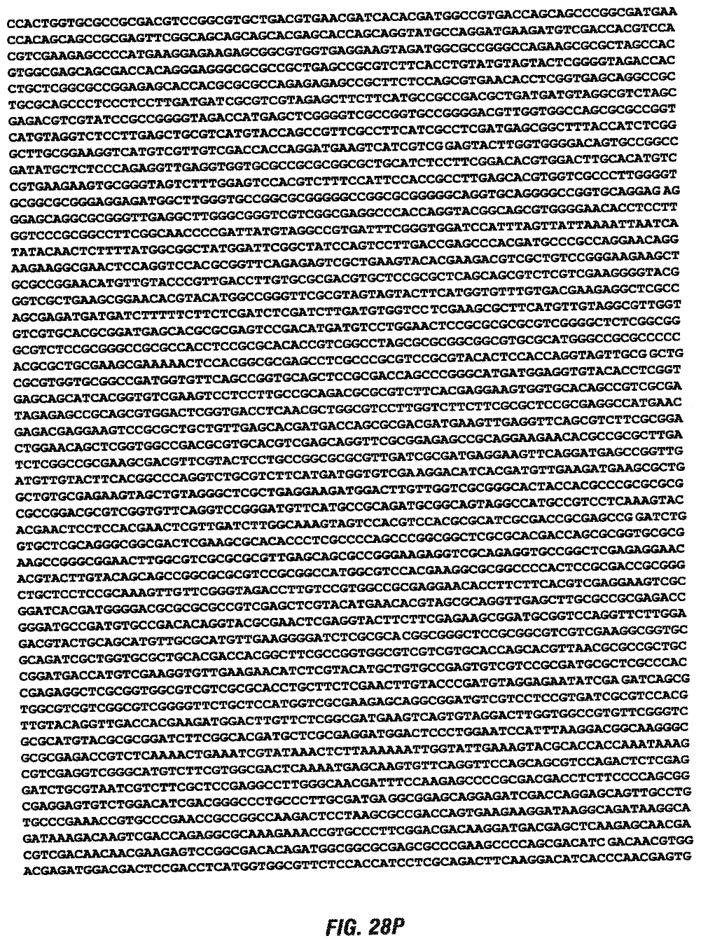

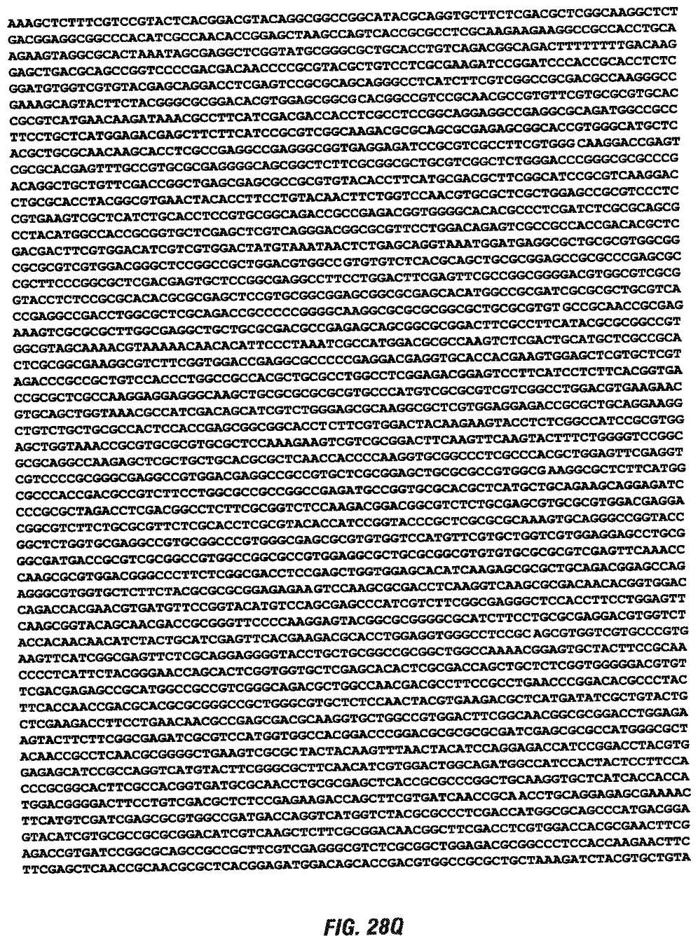

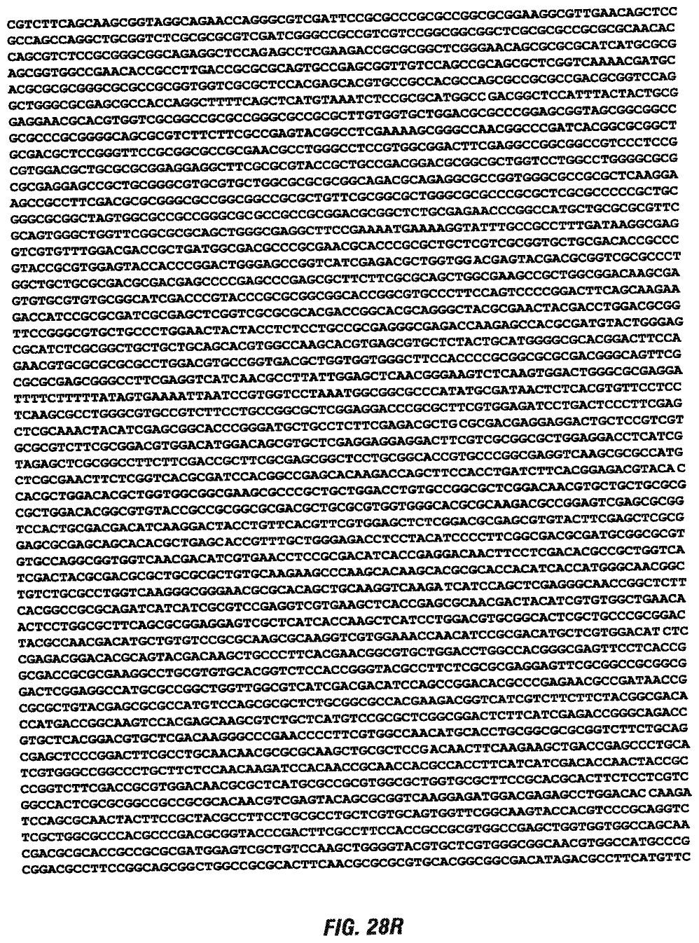

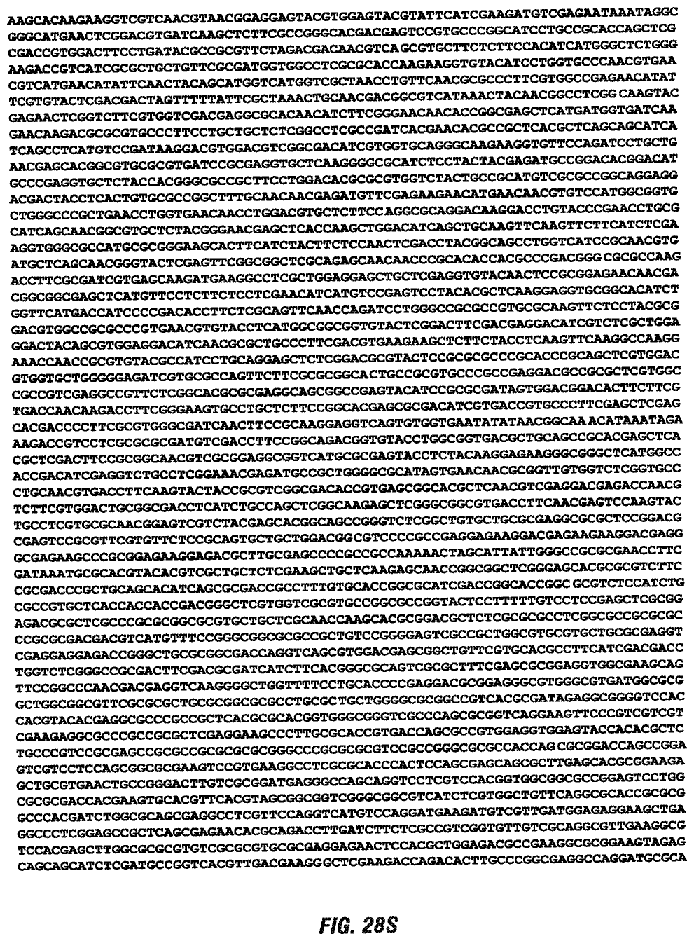

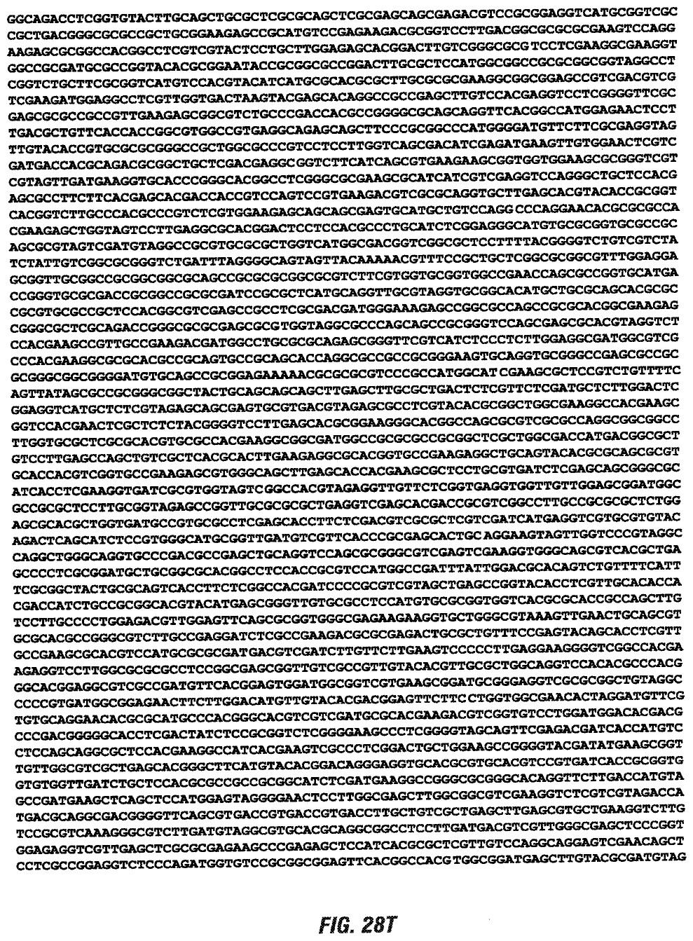

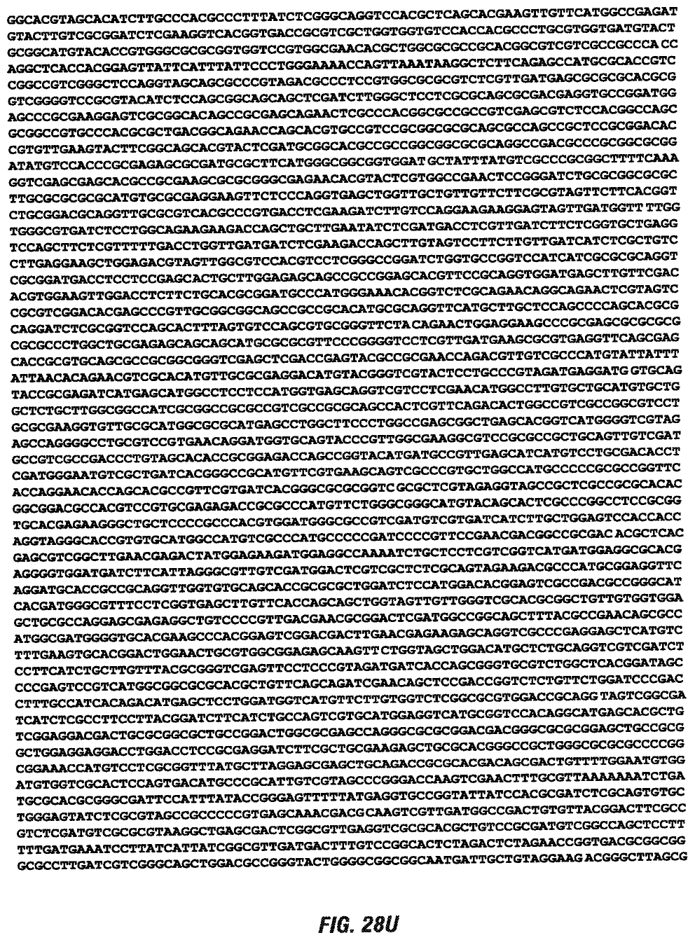

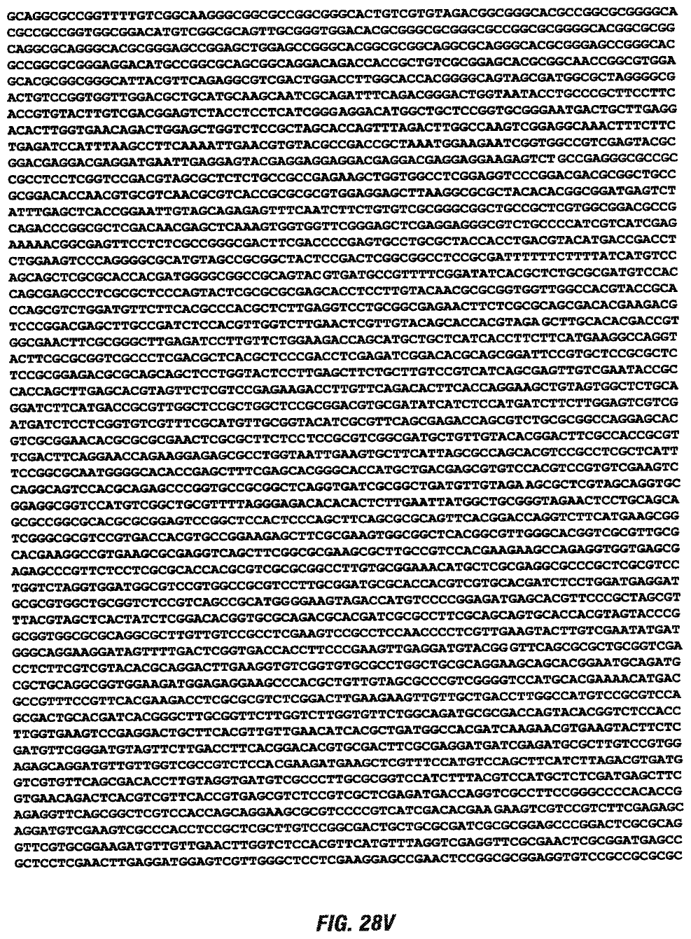

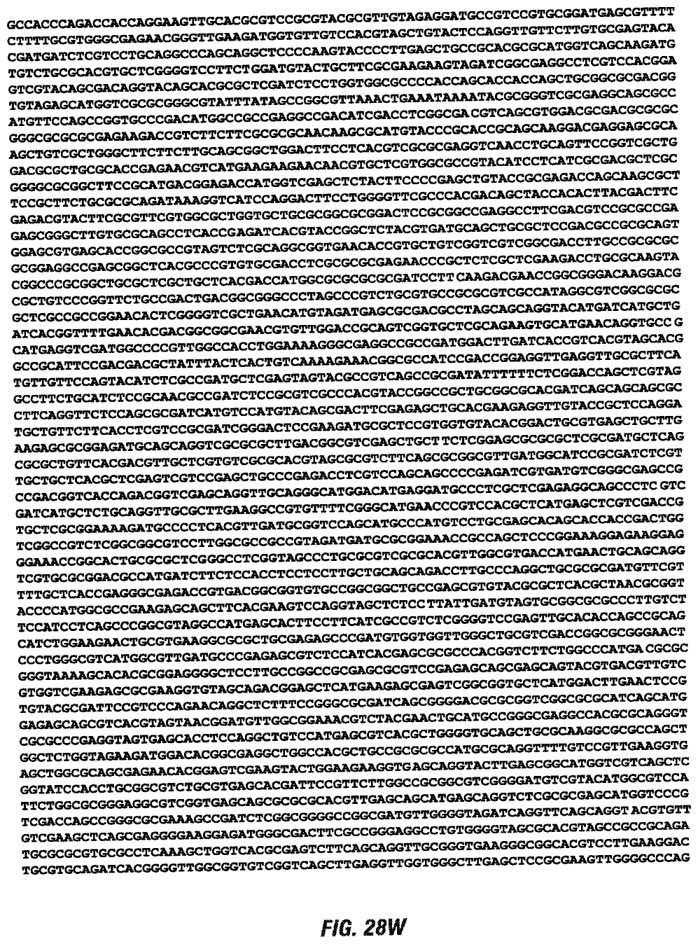

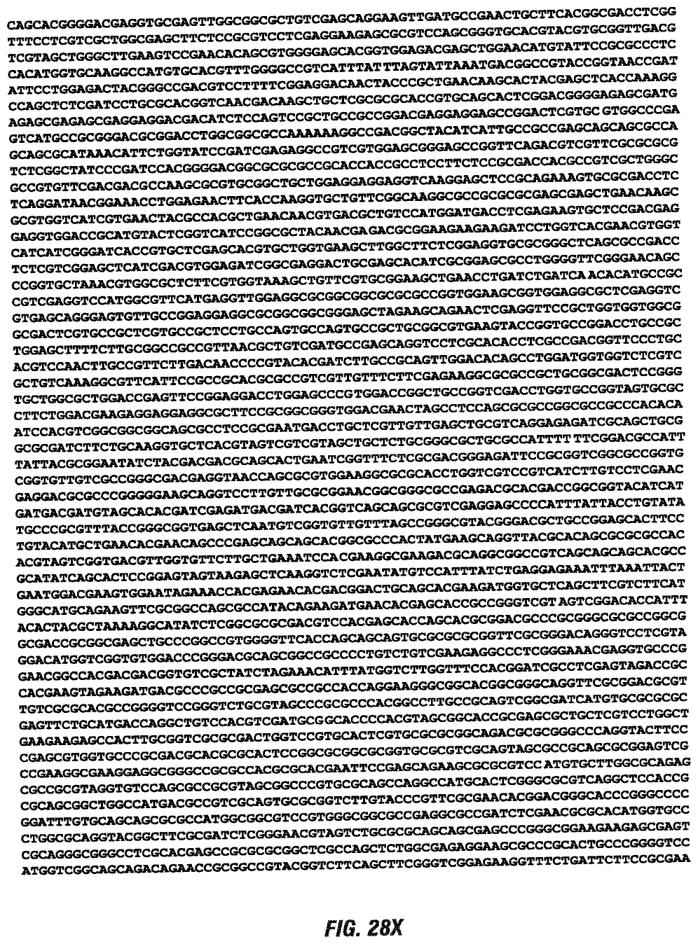

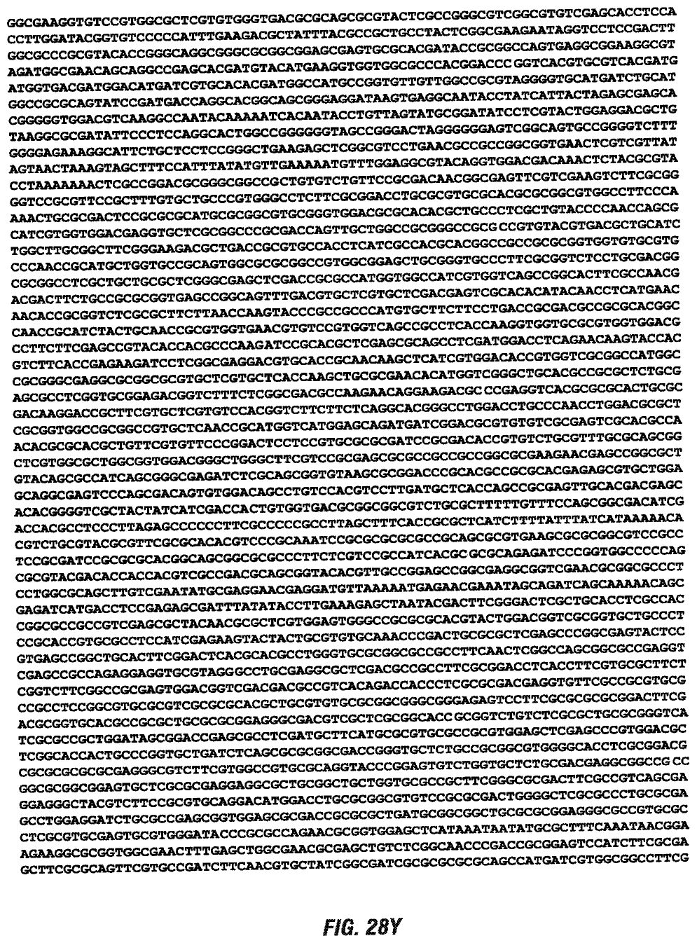

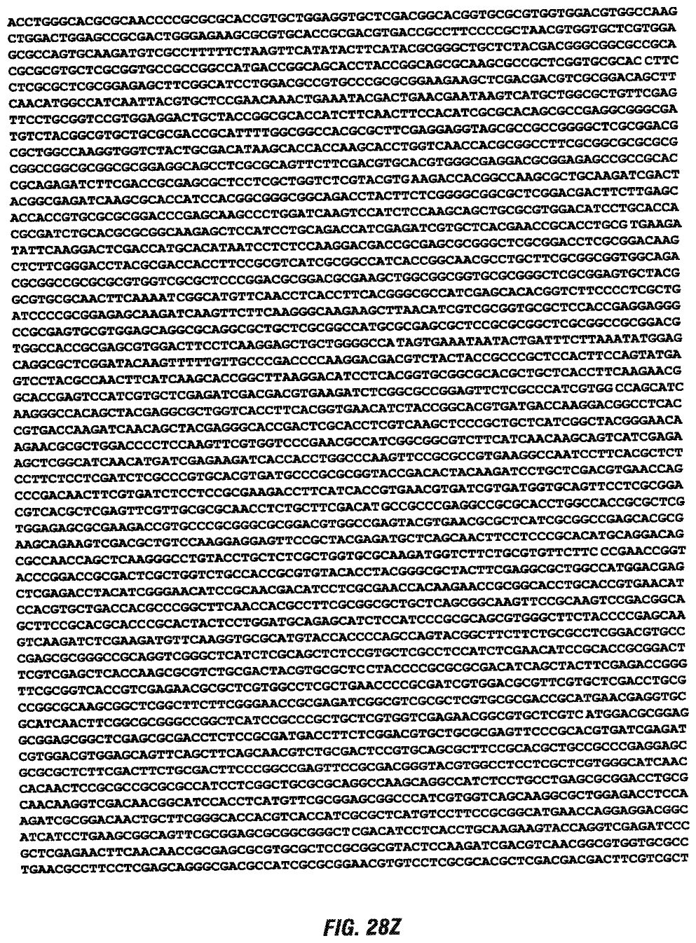

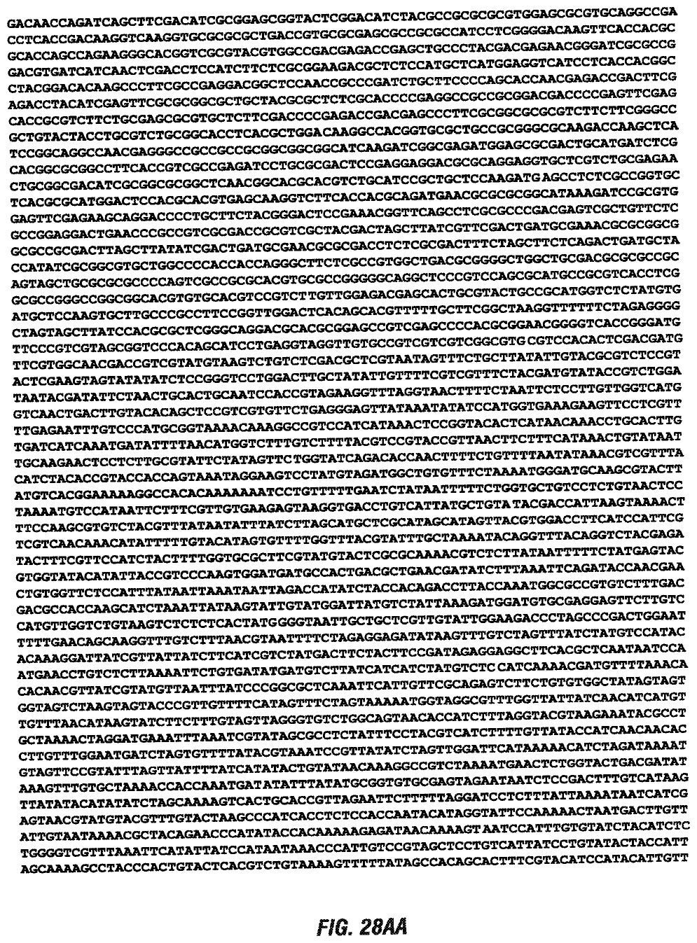

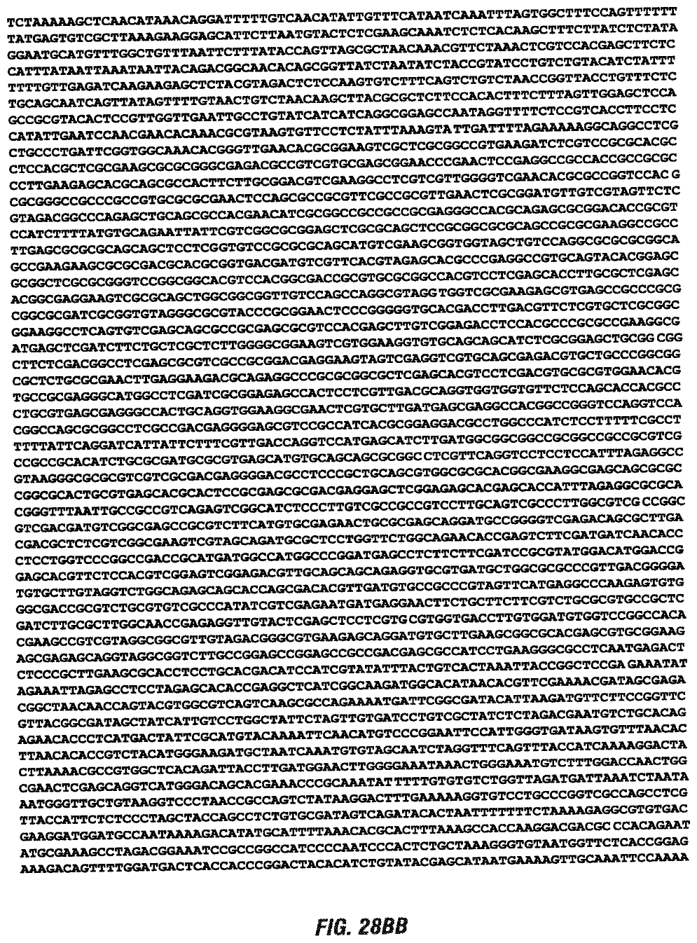

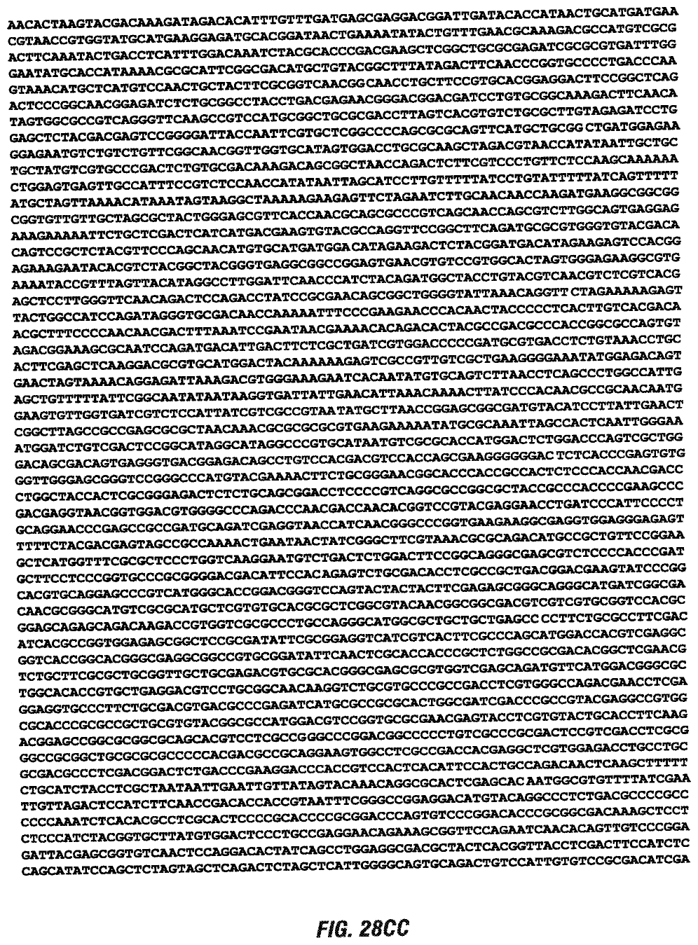

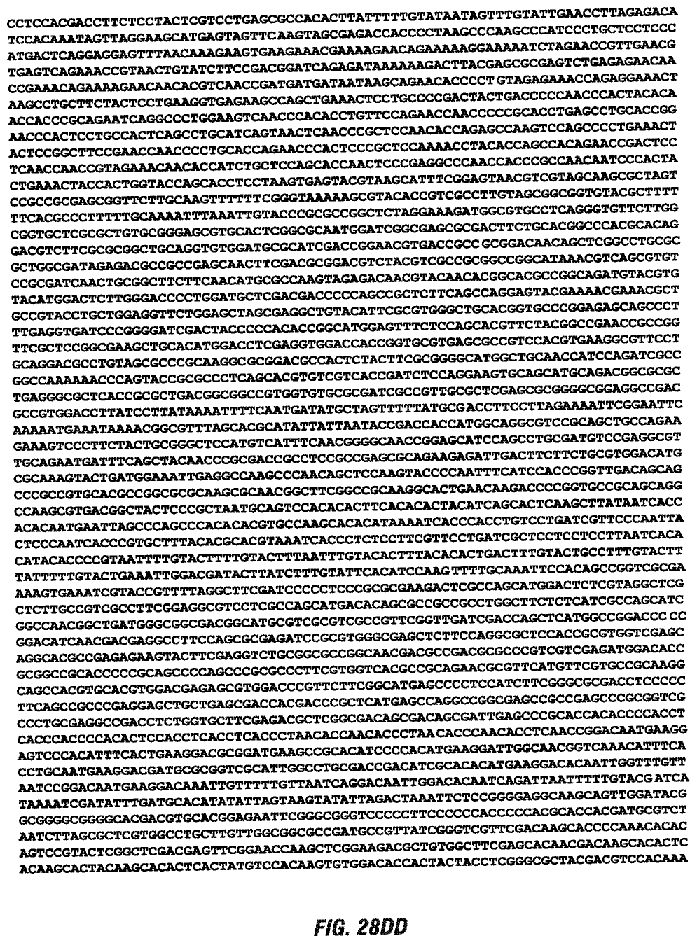

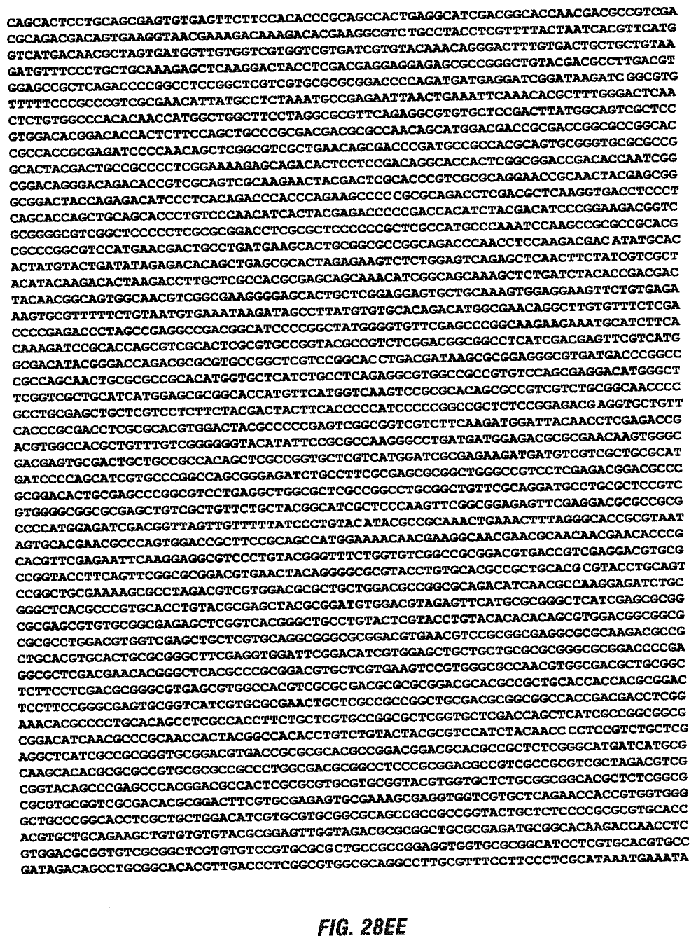

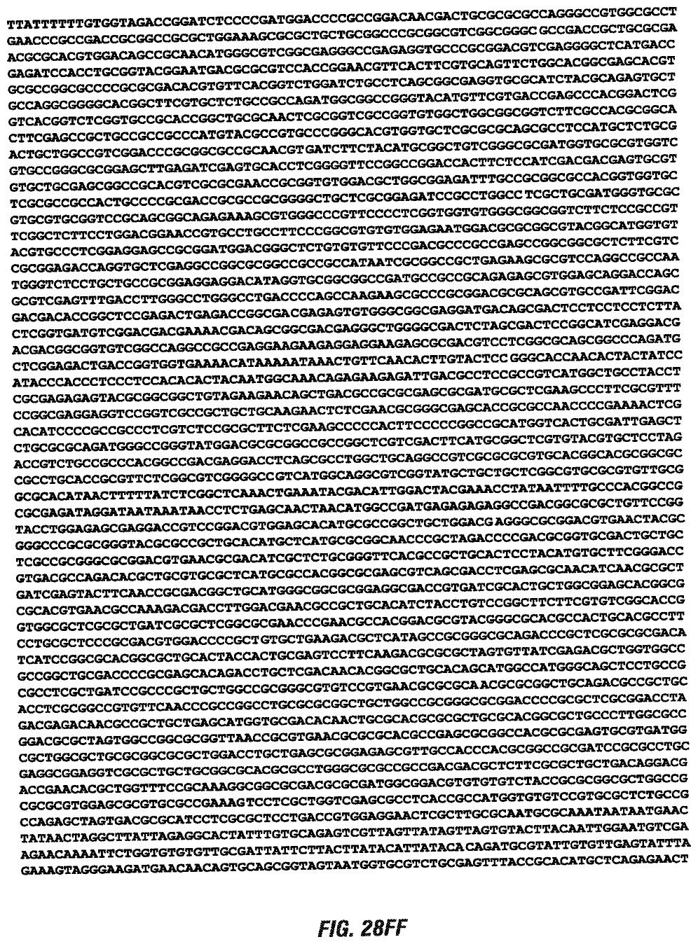

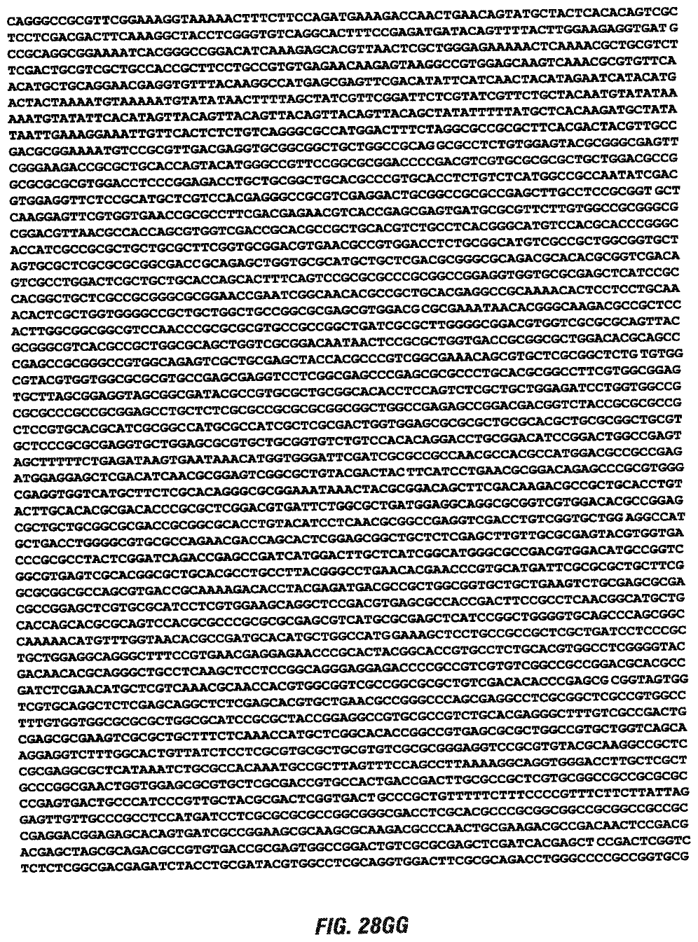

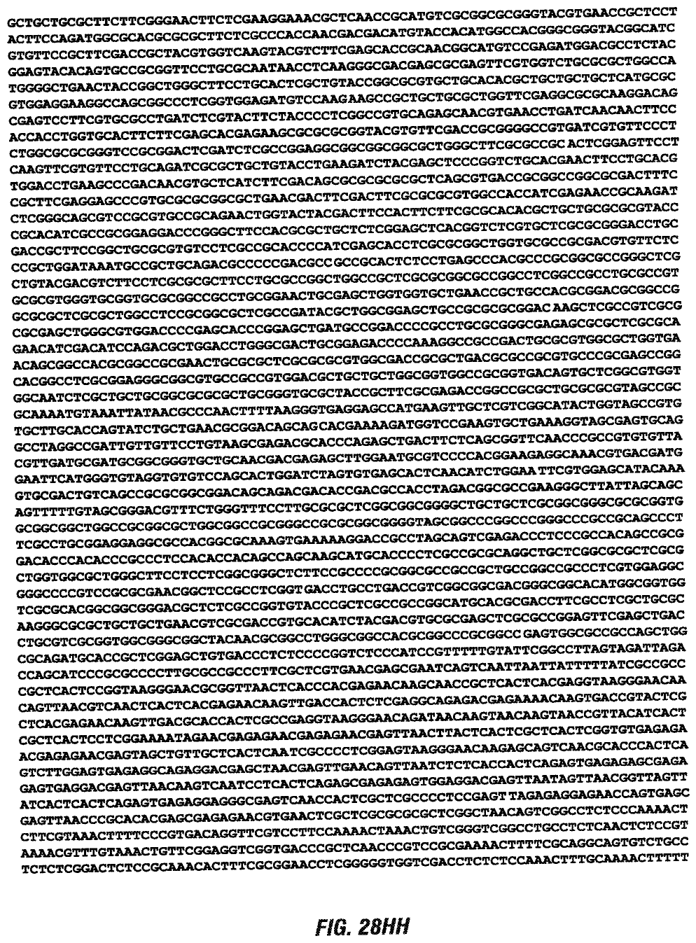

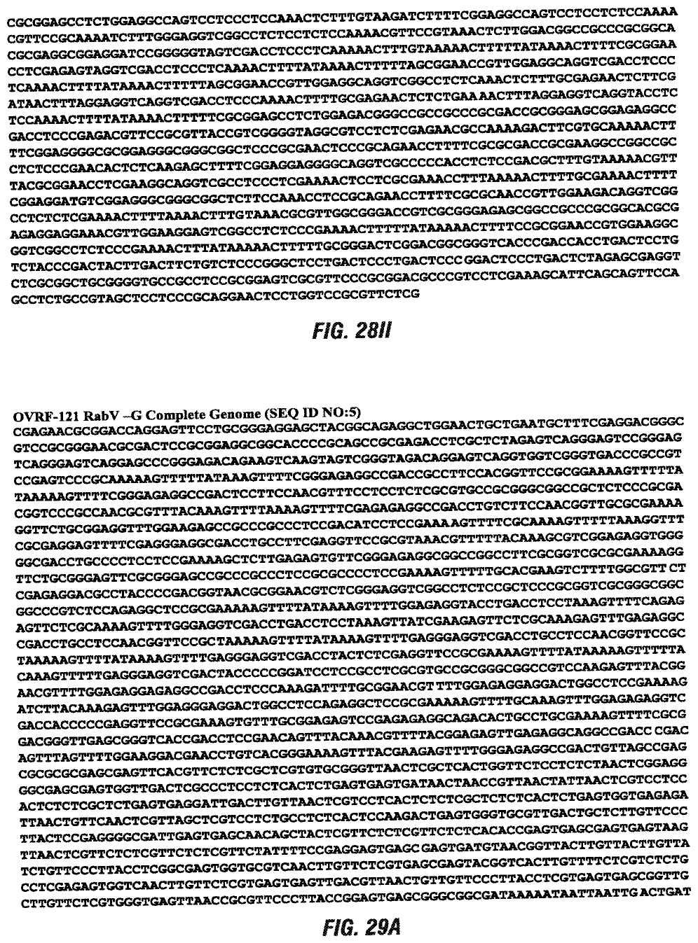

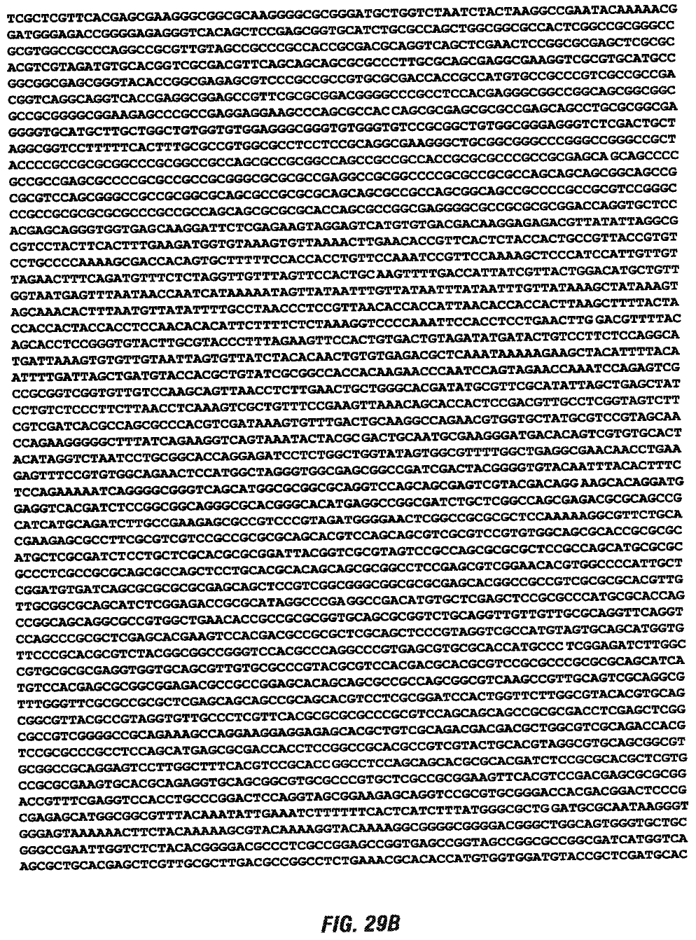

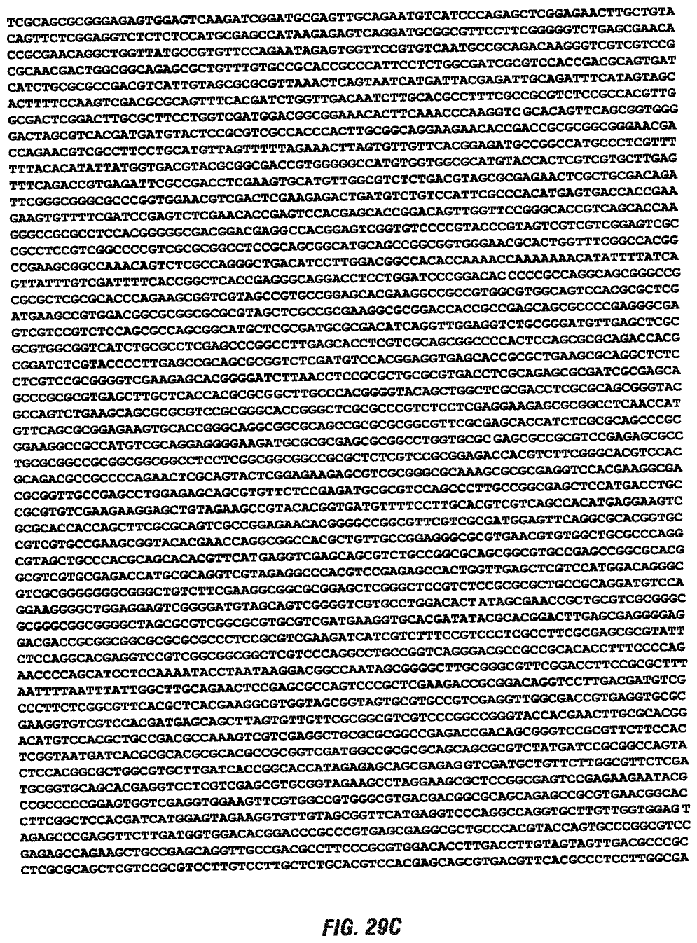

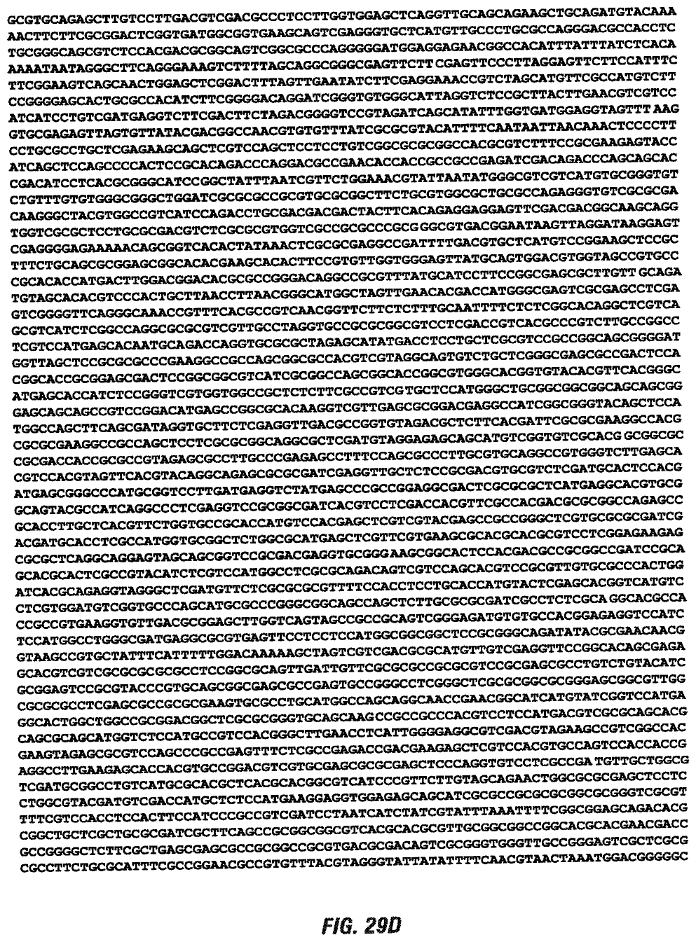

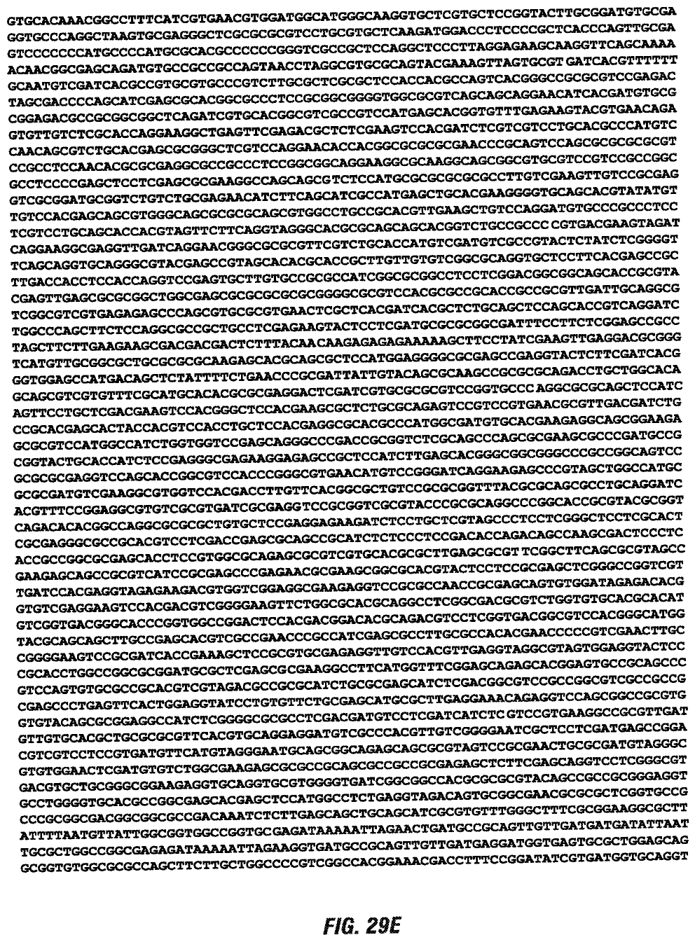

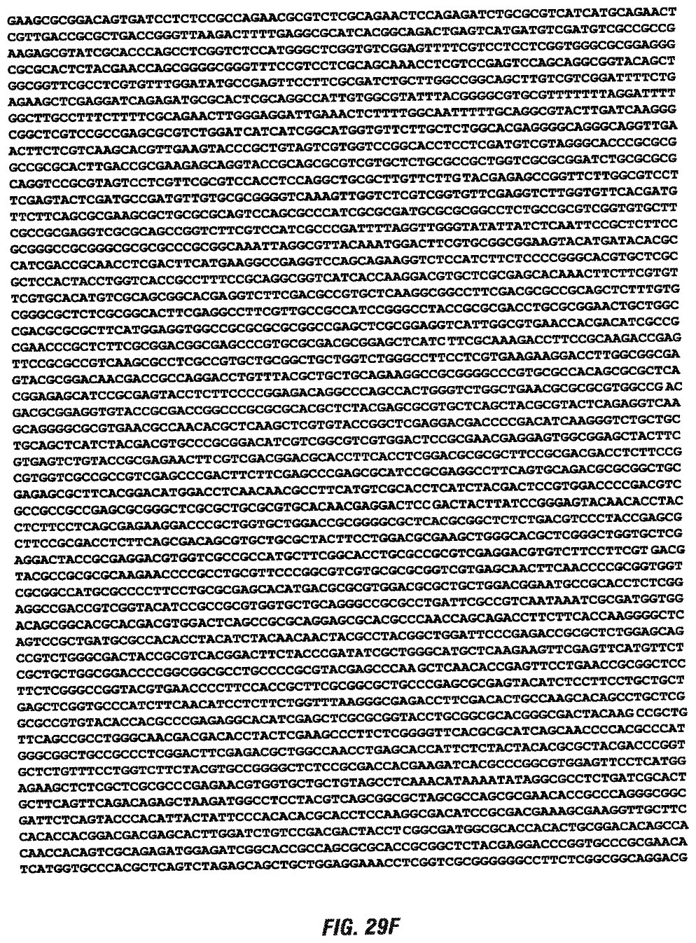

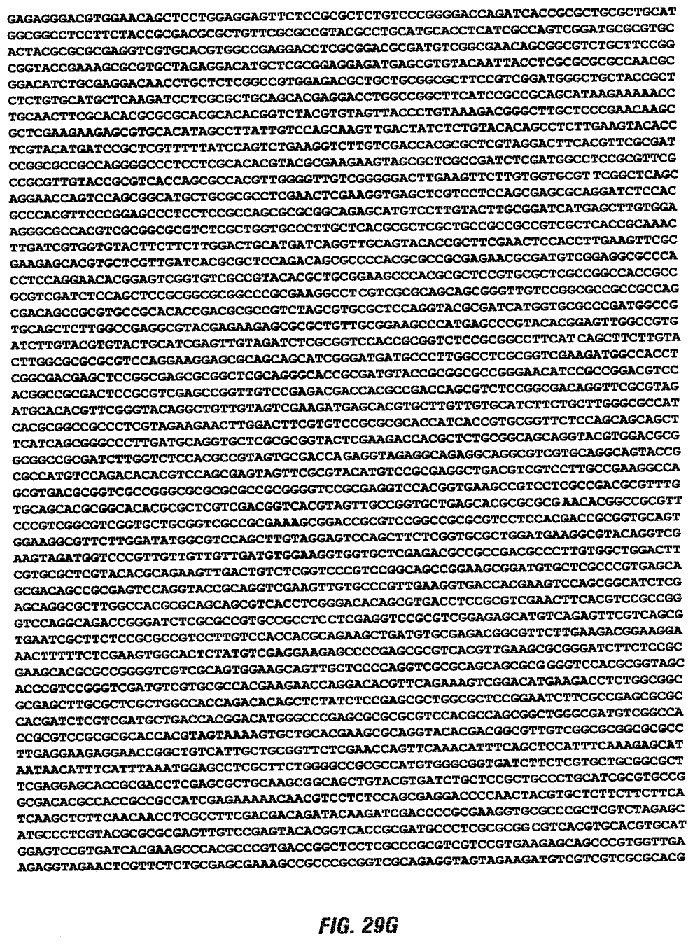

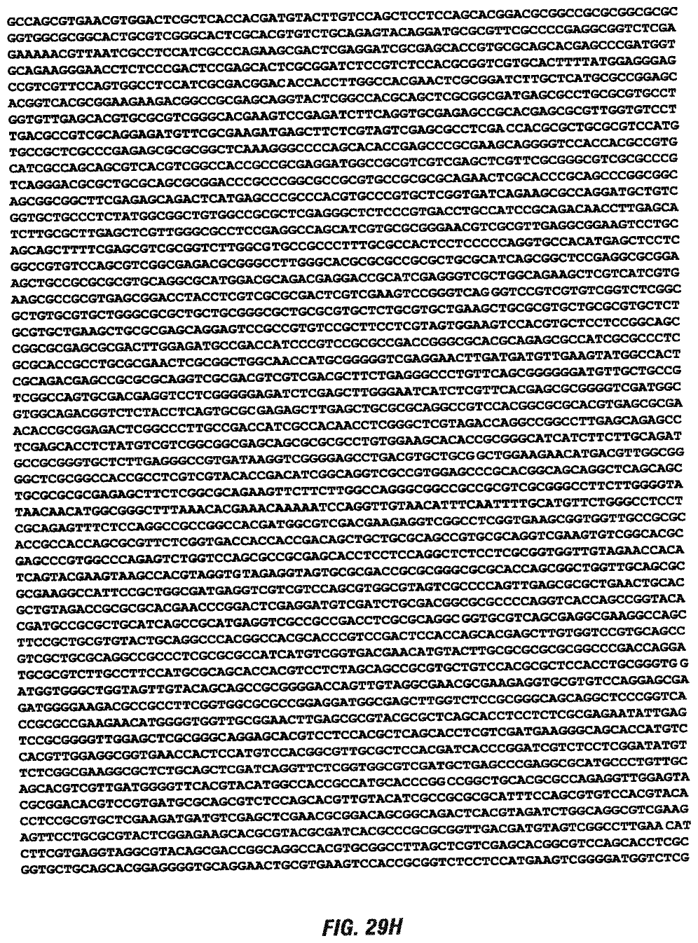

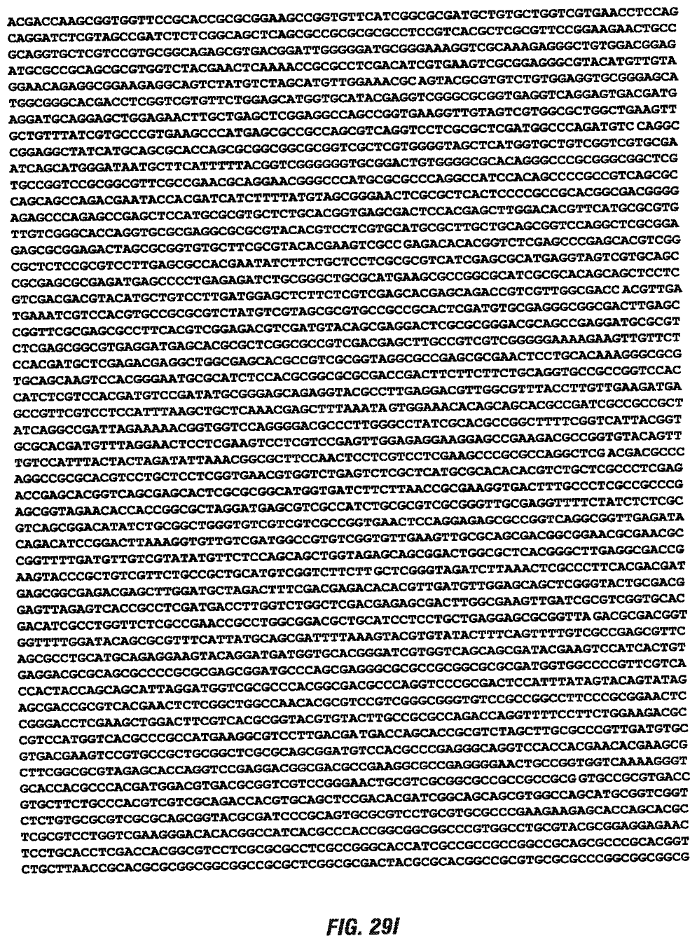

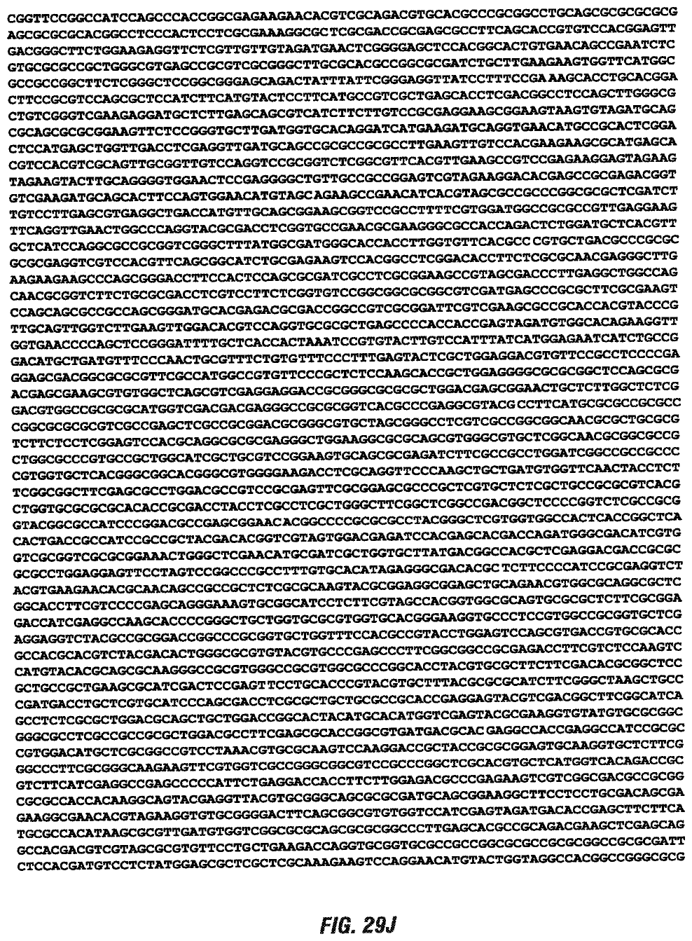

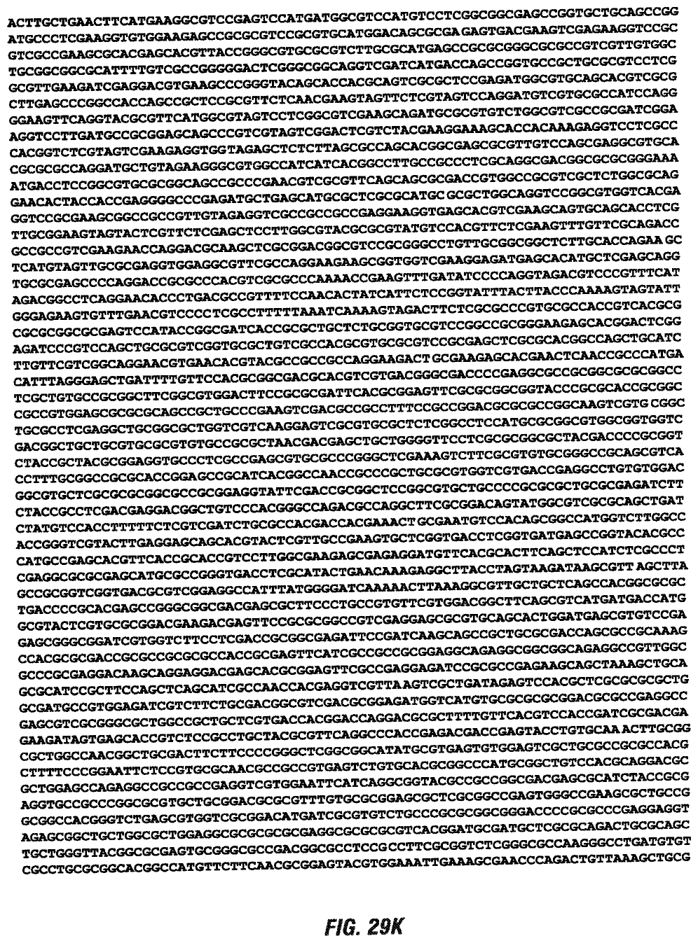

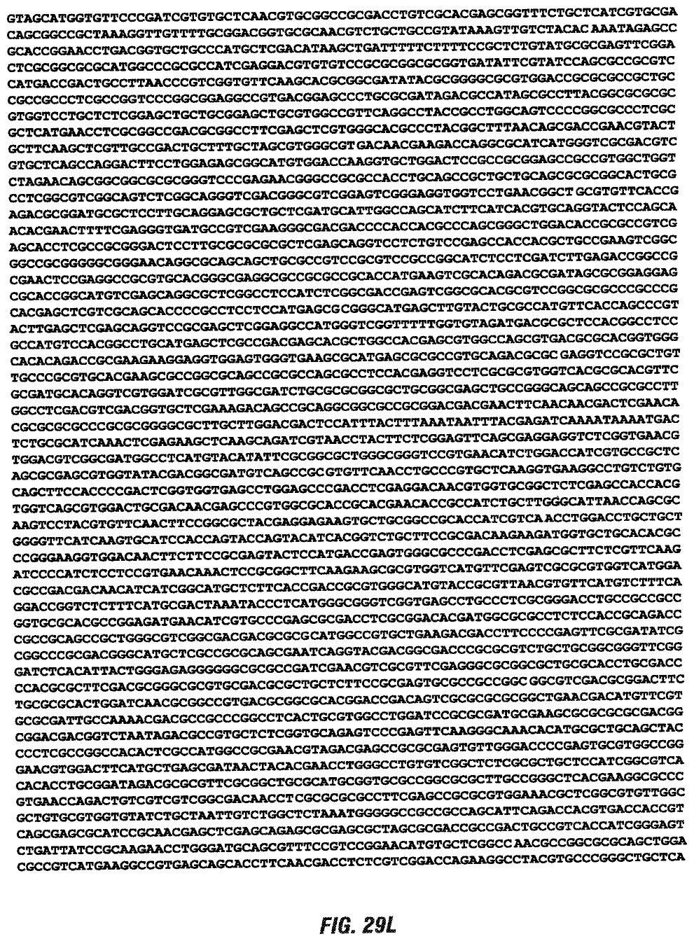

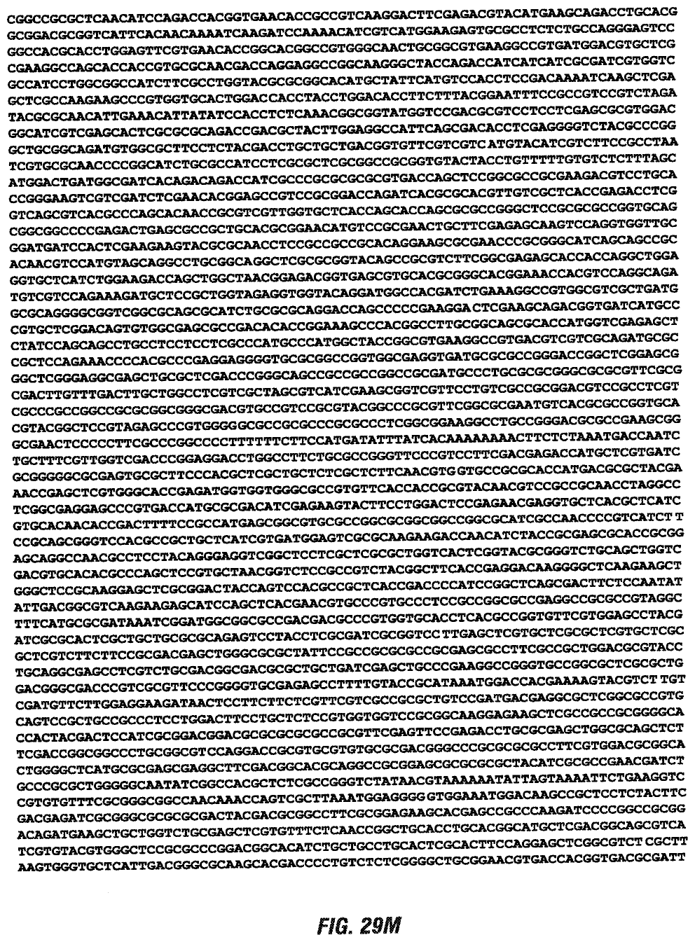

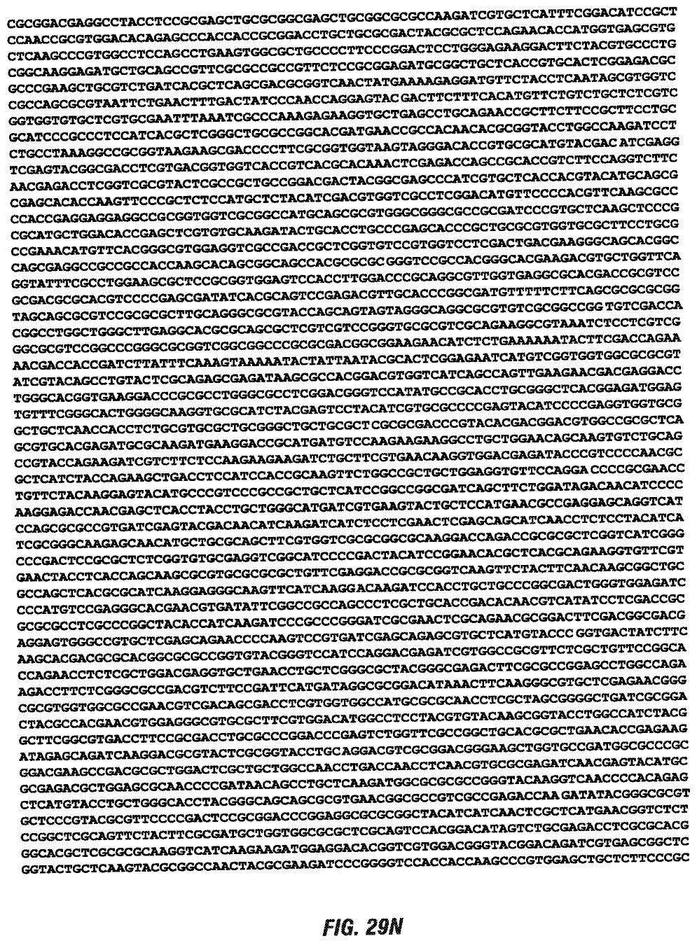

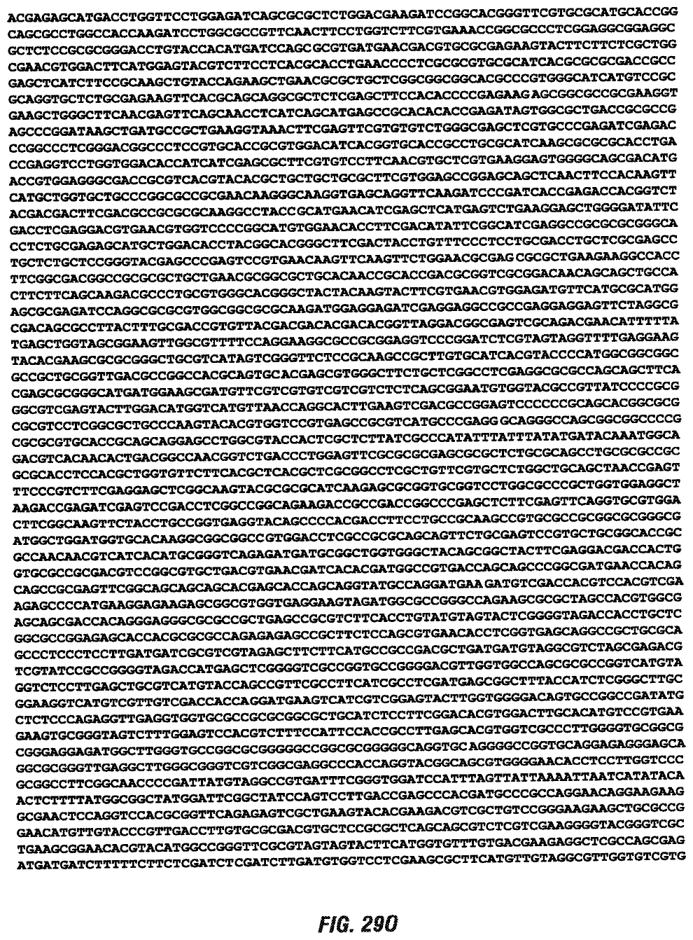

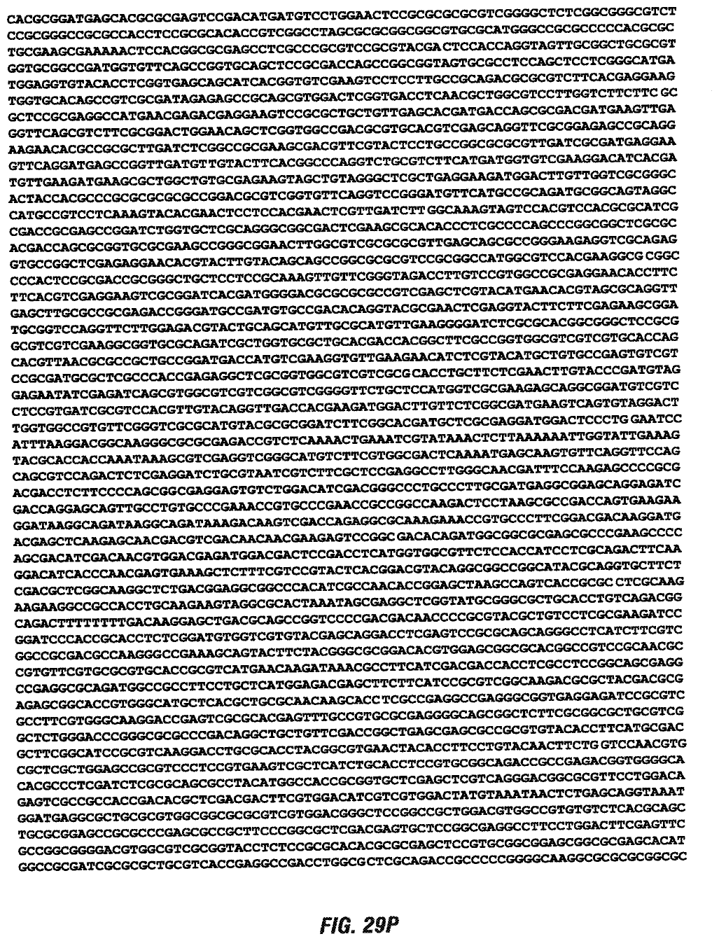

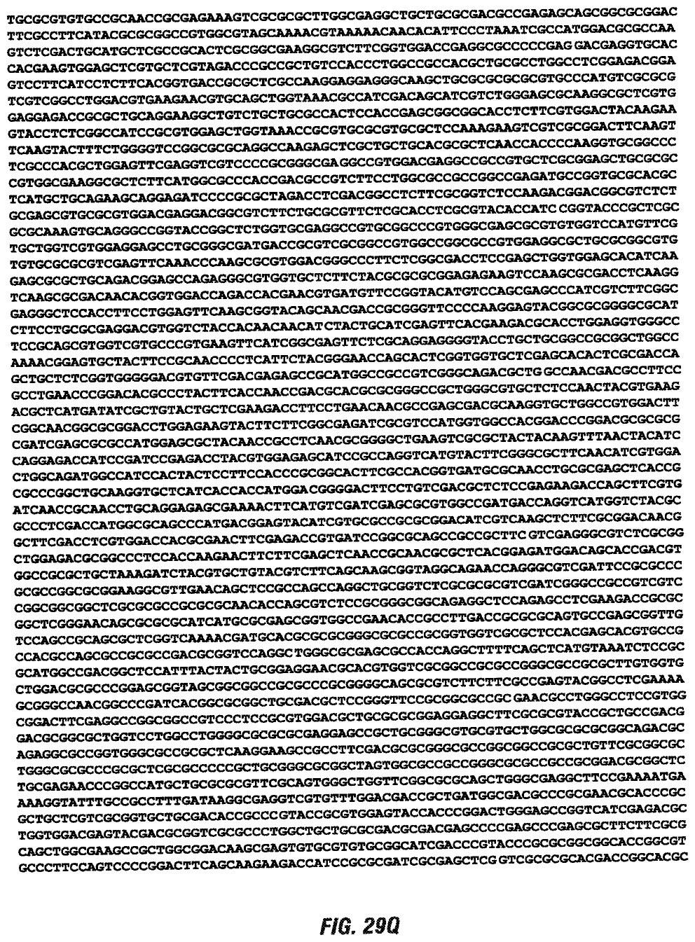

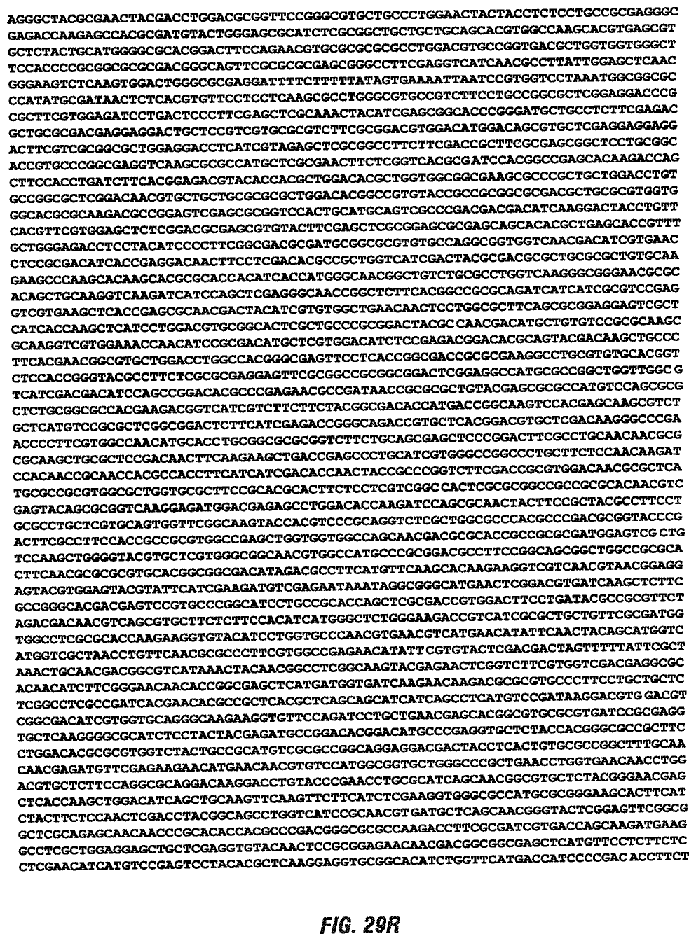

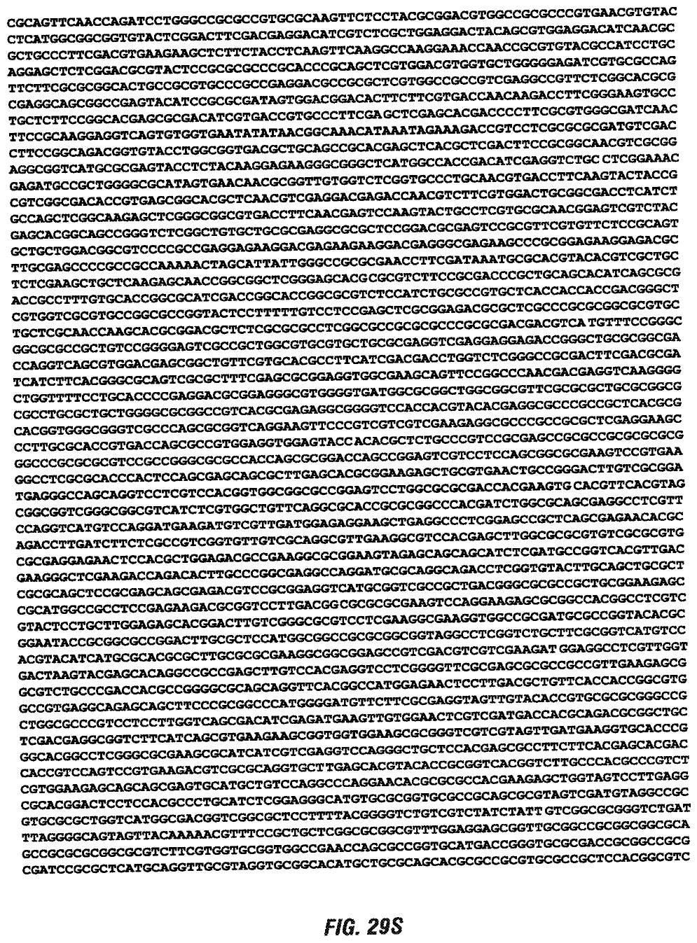

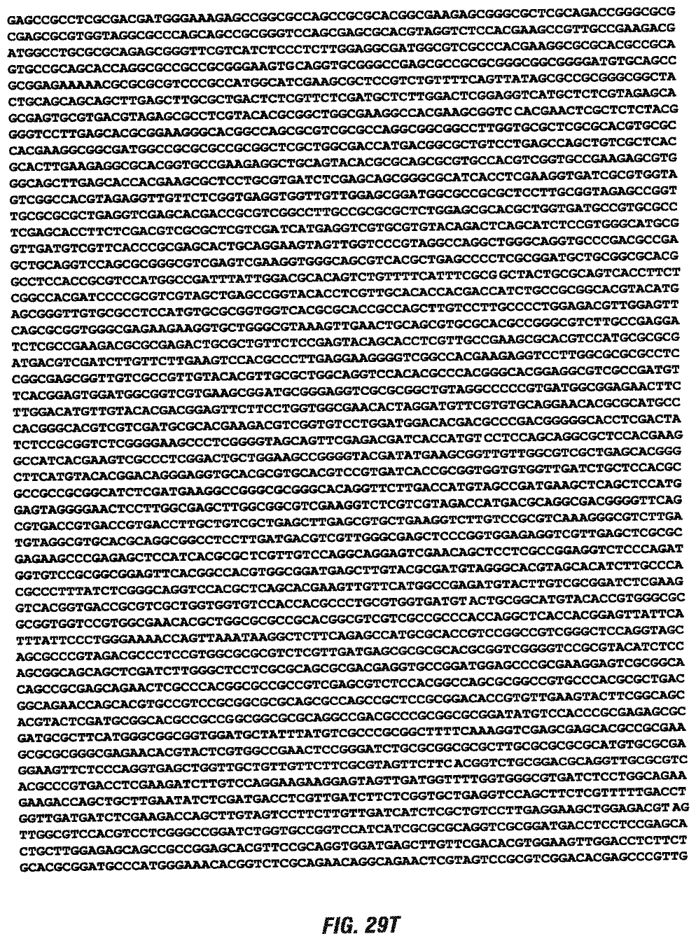

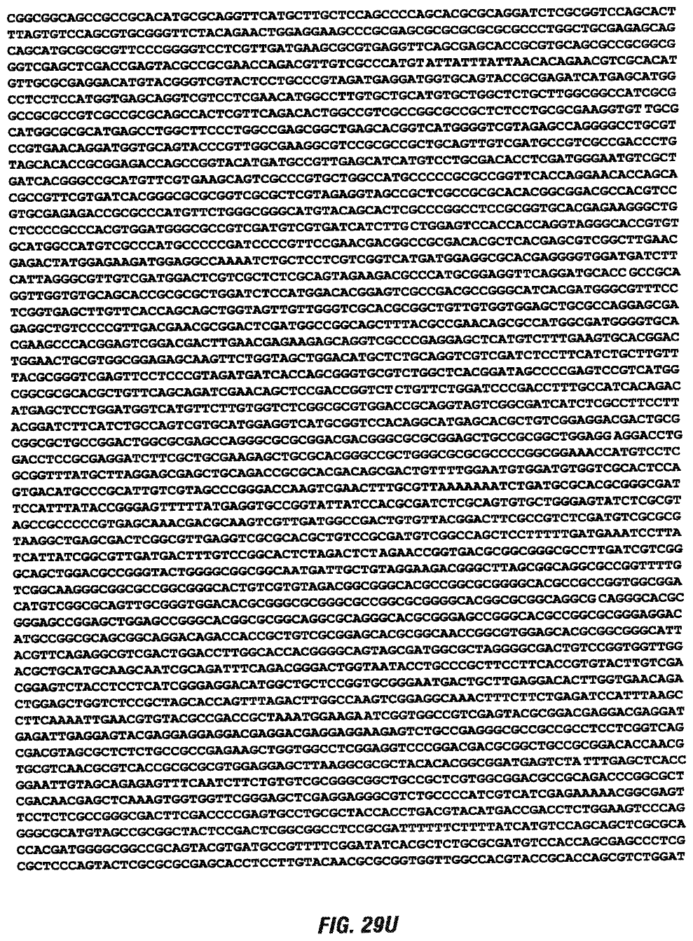

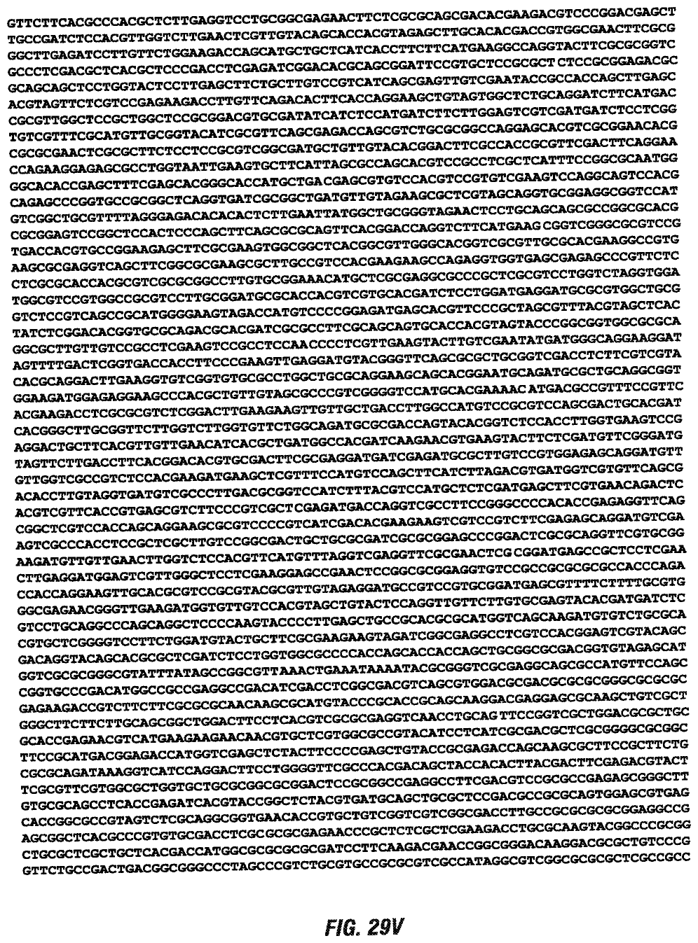

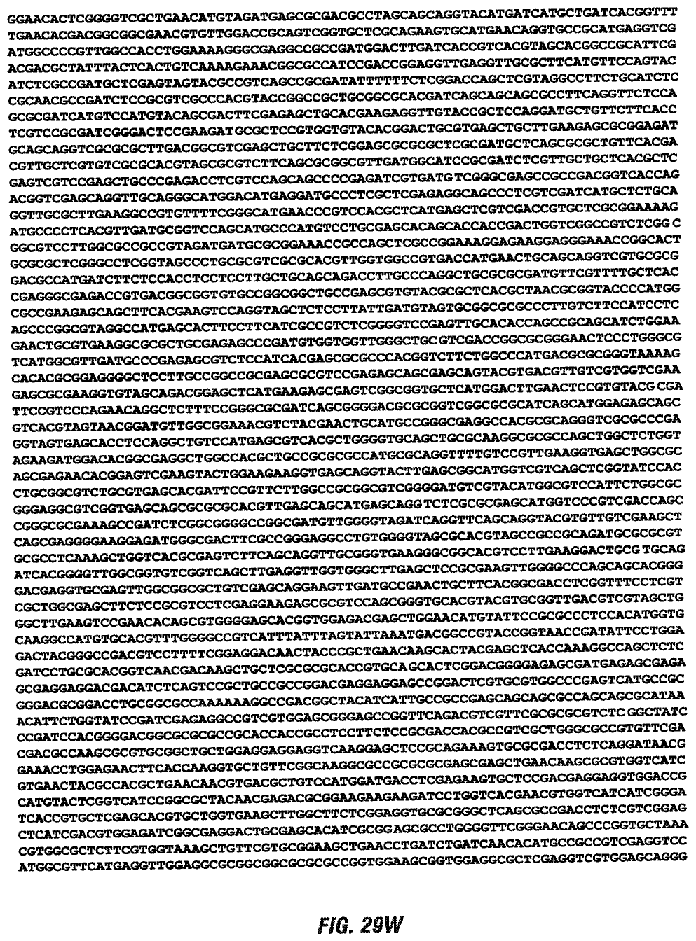

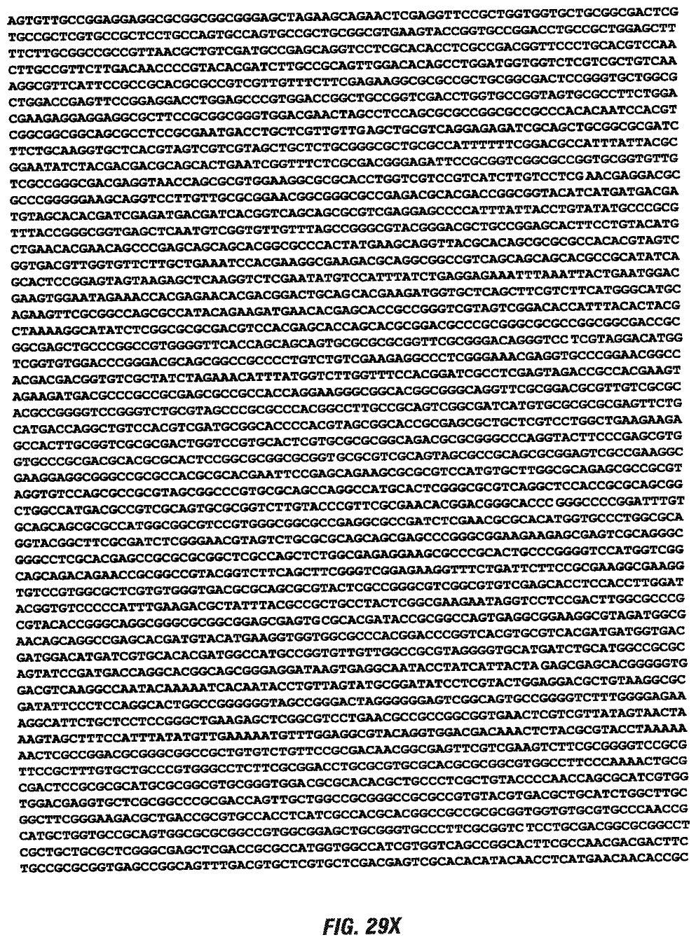

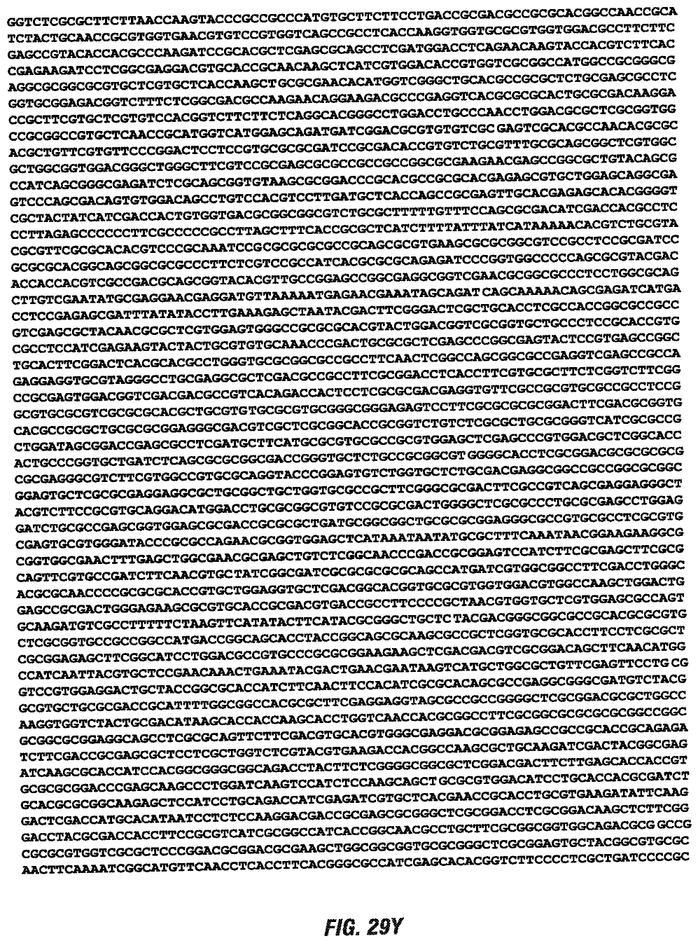

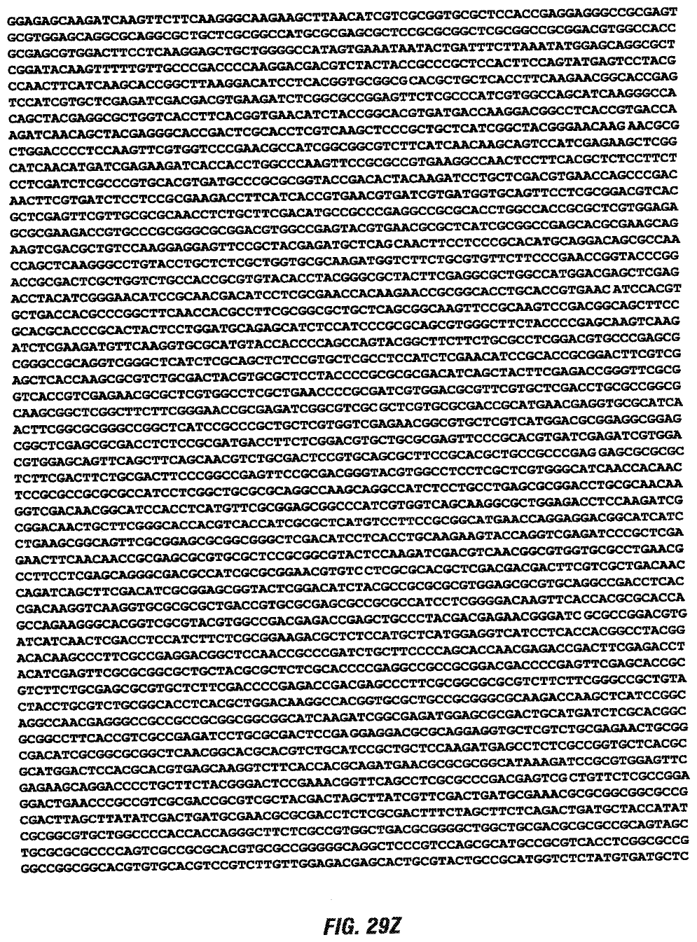

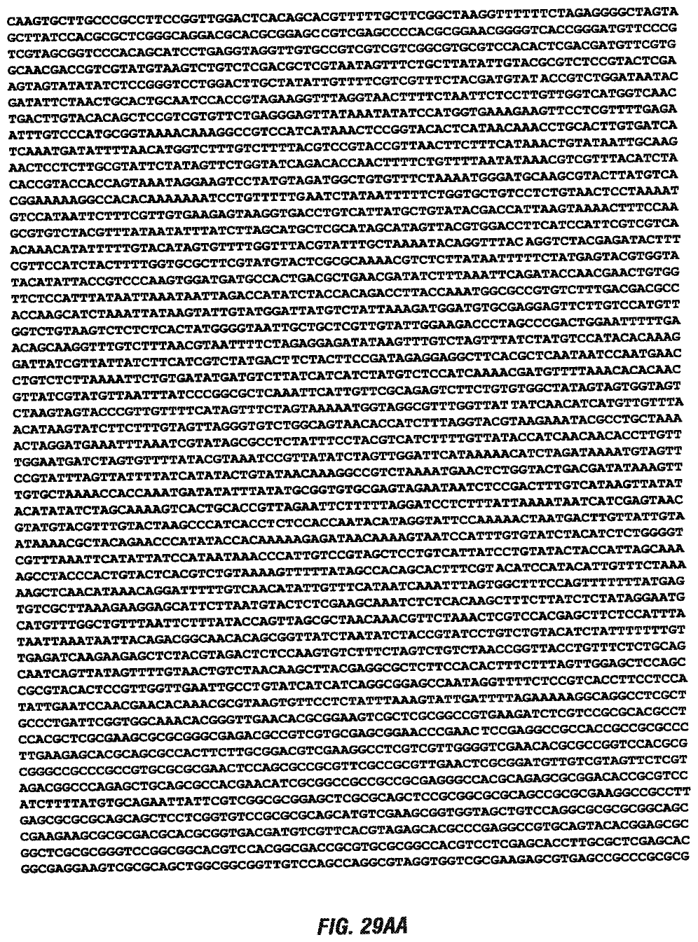

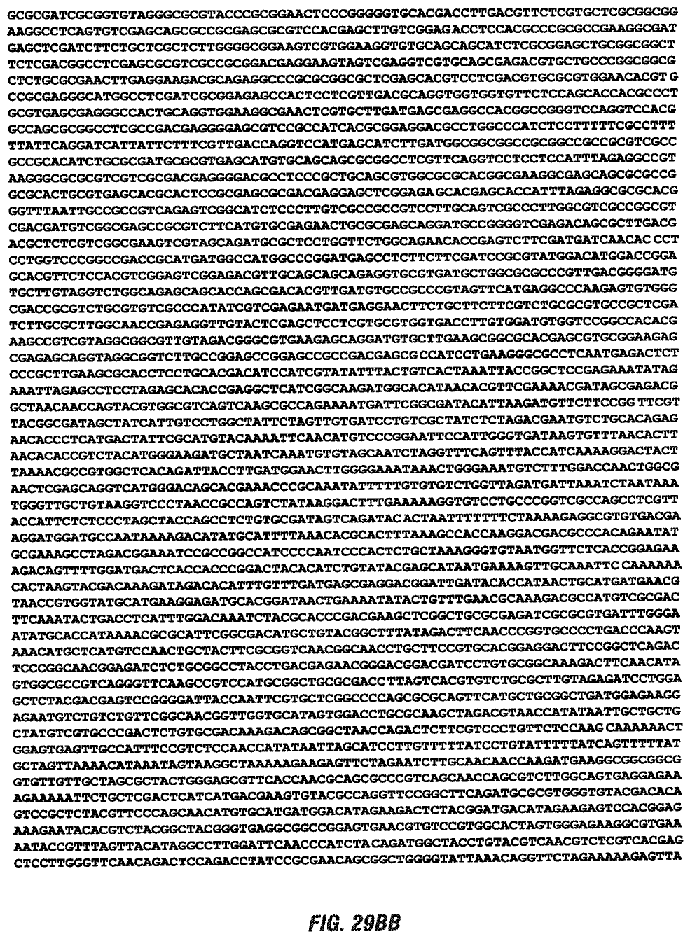

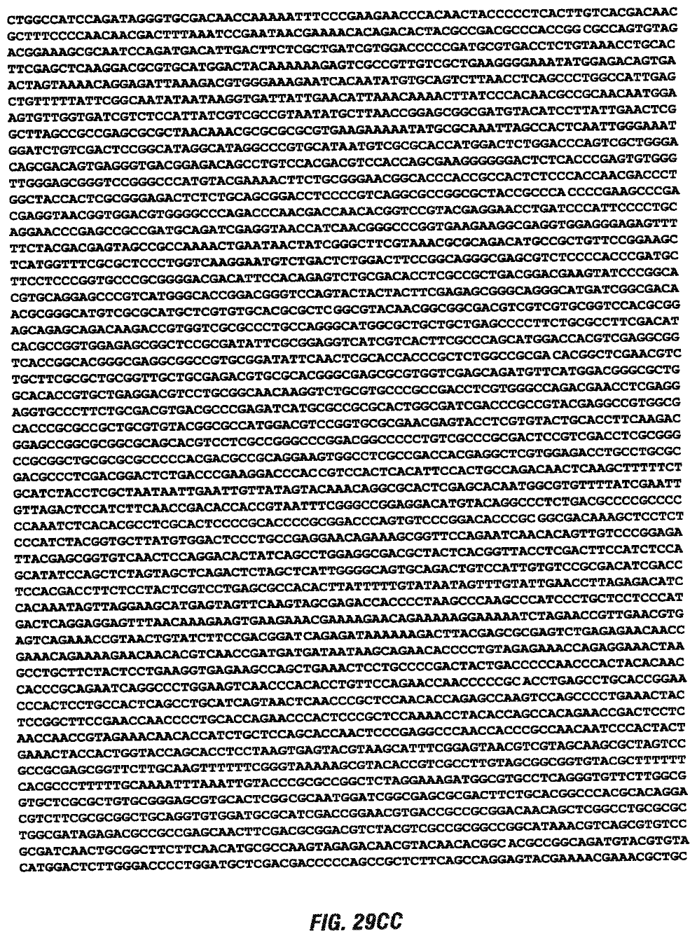

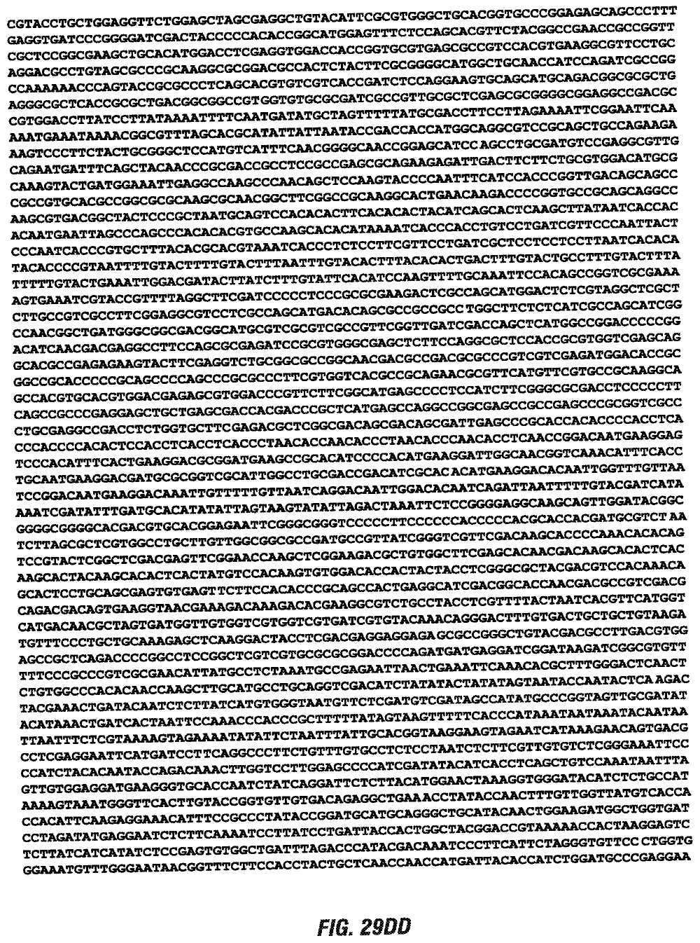

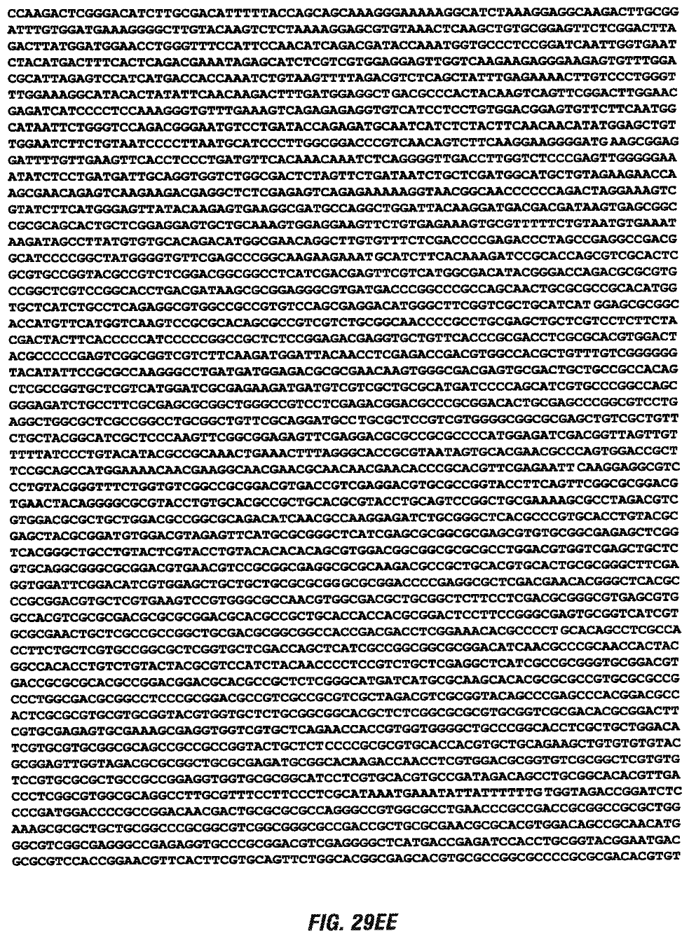

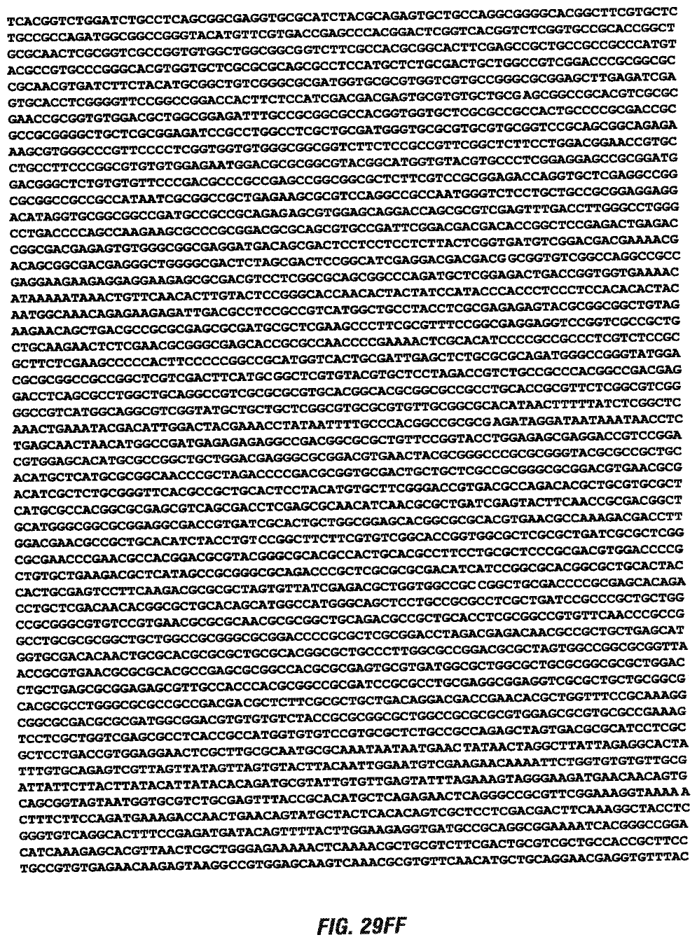

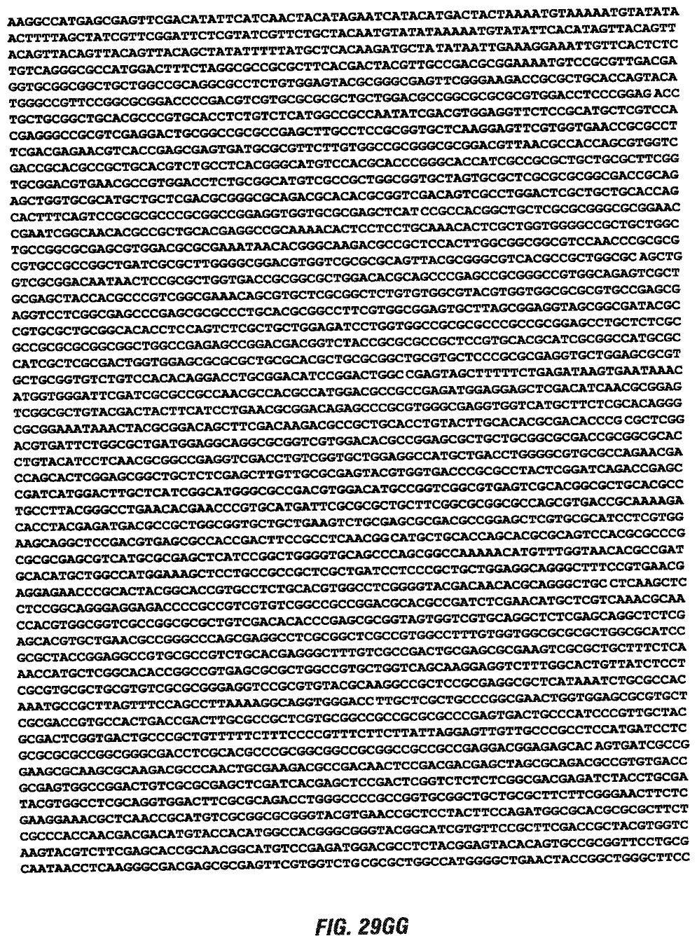

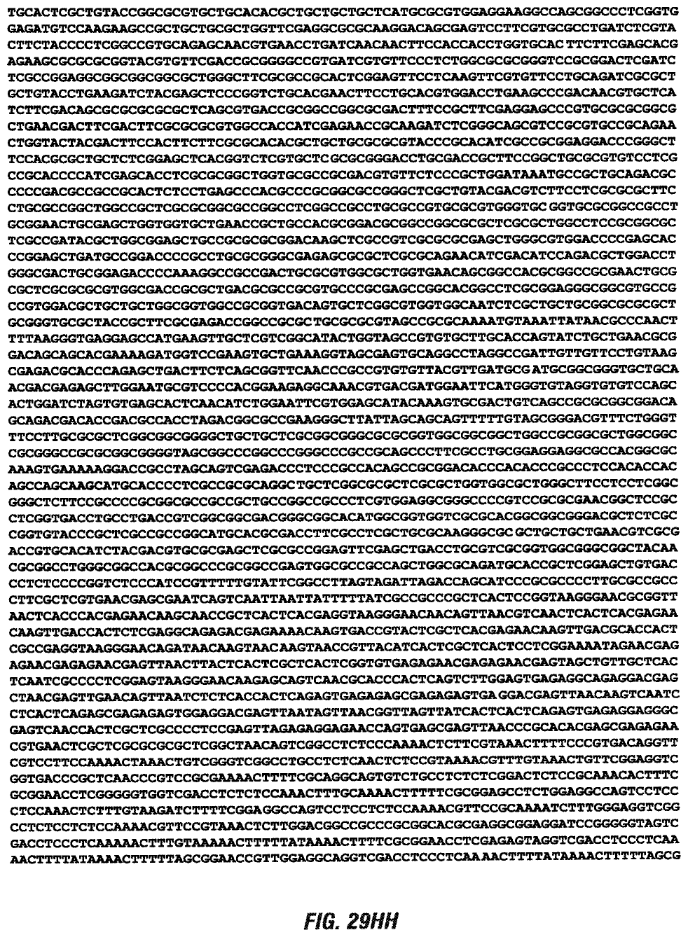

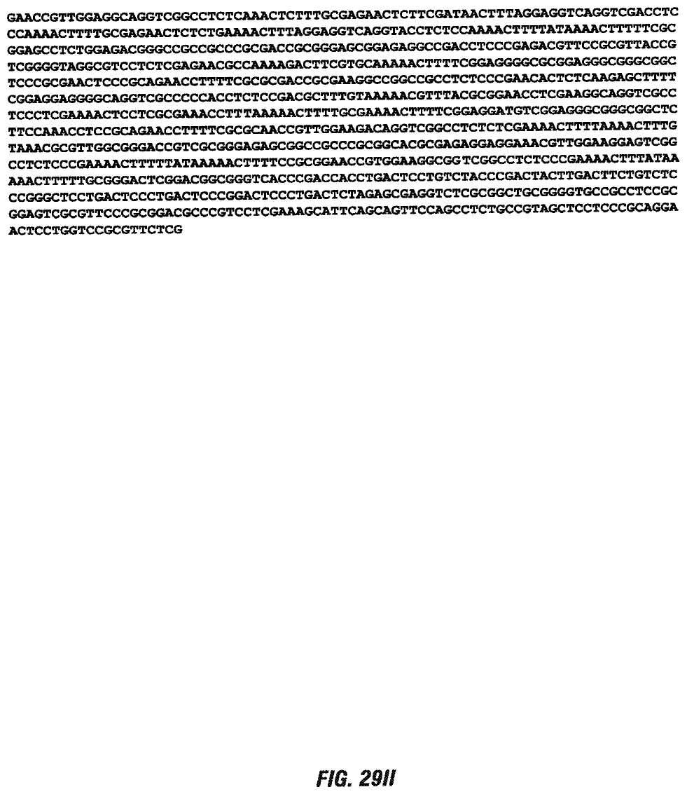

TABLE 12
(see FIG. 17)

| Cell Density | | Average | | TGI | |
|---|---|---|---|---|---|
| 3 × 10⁶/ml | Animal | Size | TGI | analysis | P-value |
| Day 20 | Number | Tumor | (%) | (TGI > 60%) | (ANOVA) |
| Control (PBS) | 6 | 4128.23 | — | — | — |
| v617a | 6 | 1552.86 | 62 | 4/6 | 0.008** |
| (1.0*10⁸ pfu/ml) | | | | | |
| v618a | 6 | 2494.45 | 40 | 3/6 | 0.078 |
| (1.0*10⁸ pfu/ml) | | | | | |
| POV-601-1A1 | 6 | 2092.23 | 50 | 3/6 | 0.030* |
| (v601-1A1) | | | | | |
| (1.0*10⁸ pfu/ml) | | | | | |

v617a: the mutant with a complete deletion of the ORFV111 gene
v618a: the mutant with a complete deletion of the coding region in the ORFV111 gene

Material Deposit

The following material has been deposited with the China Center for Type Culture Collection in accordance with the provisions of the Budapest Treaty (CCTCC) (Wuhan University, Wuhan, China, 430072):

| | CCTCC Deposit | Deposit |
|---|---|---|
| Material | Number | Date |
| The ORFV Mutant Virus | V202029 | 2020 May 19 |
| POV-601-1A1 (v601-1A1) | | |

### REFERENCES

1. Hanahan, D., Weinberg, R.A. (2011). Hallmarks of cancer: the next generation. Cell, 144(5):646-74.
2. Miest, T. S., Cattaneo, R. (2014). New viruses for cancer therapy: meeting clinical needs. Nat Rev Microbiol, 12(1):23-34.
3. Burke, J., Nieva, J., Borad, M. J., Breitbach, C. J. (2015). Oncolytic viruses: perspectives on clinical development. Curr Opin Virol, 13:55-60.
4. Seymour, L. W., Fisher, K. D. (2016). Oncolytic viruses: finally delivering. Br J Cancer, Feb 16; 114(4):357-361.
5. Harrington, K. J., Puzanov, I., Hecht, J. R., Hodi, F. S., Szabo, Z., Murugappan, S., Kaufman, H. L. (2015). Clinical development of talimogene Laherparepvec (T-VEC): a modified herpes simplex virus type-1-derived oncolytic immunotherapy. Expert Rev Anticancer Ther, (12):1389-1403.

### 6. Yin, Z. and Liu, J. H. (1997) Animal Virology (Second Edition), Science Press, 977-978

7. Wang, Z. J. (2018) Research, development and quality control of Biopharmaceuticals (third edition), Science Press.

### 8. CDC (2006). Orf Virus Infection in Humans-New York, Illinois, California, and Tennessee, 2004-2005. Morbidity and Mortality Weekly Report, 55(3):65-68.

9. Kim, D. H., Thorne, S. H. (2009). Targeted and armed oncolytic poxviruses: a novel multi-mechanistic therapeutic class for cancer. Nat Rev Cancer, 9:64-71.
10. McFadden, G. (2005). Poxvirus tropism. Nat Rev Microbiol, 3(3):201-213.
11. Wang, R., Wang, Y., Liu, F., Luo, S. (2018). Orf virus: A promising new therapeutic agent. Rev Med Virol, e2013.
12. Rintoul, J. L., Lemay, C. G., Tai, L. H., Stanford, M. M., Falls, T. J., Bridle, B. W., Souza, C. T., Daneshmand, M., Ohashi, P. S., Wan, Y., Lichty, B. D., Mercer, A. A., Auer, R. C., Atkins, H. L., Bell, J. C. (2012). ORFV: a novel oncolytic and immune stimulating parapoxvirus therapeutic. Mol Ther, 20(6):1148-1157.
13. Seet, B. T., McCaughan, C. A., Handel, T. M., Mercer, A., Brunetti, C., McFadden, G., Fleming, S. B. (2003). Analysis of an orf virus chemokine-binding protein: shifting ligand specificities among a family of poxvirus viroceptors. Proceedings of the National Academy of Sciences, 100(25):15137-15142.
14. Bergqvist, C., Kurban, M., Abbas, O. (2017). Orf virus infection. Rev Med Virol, 27(4).
15. Liu, W., Yang, K. K., Yin, D. D., Wang, Y. H., Yu, Z. R., Jiang, S. D., Li, C. F., Li, Y. D., Wang, Y. (2018). Prokaryotic expression and subcellular localization of dUTPase gene of orf virus, Chinese Veterinary Science, 7:818-823
16. Irwin, C. R., Hitt, M. M., Evans, D. H. (2017). Targeting Nucleotide Biosynthesis: A Strategy for Improving the Oncolytic Potential of DNA Viruses. Front Oncol, 7:229.
17. Fleming, S. B., McCaughan, C., Lateef, Z., Dunn, A., Wise, I. M., Real, N. C., Mercer, A. A. (2017). Deletion of chemokine binding protein gene from the parapoxvirus orf virus reduces virulence and pathogenesis in sheep. Front Microbiol, 8:46.
18. Twumasi-Boateng, K., Jessica, L. P., Eunice Kwok, Y. Y., John, C. B., Nelson, B. H. (2018). Oncolytic viruses as engineering platforms for combination immunotherapy. Nat Rev Cancer, 18:419-432.
19. Diel, D. G., Lou, S., Delhon, G., Peng, Y., Flores, E. F., Rock, D. L. (2011). A nuclear inhibitor of NF-kappaB encoded by a poxvirus. J Virol, 85(1):264-275.

### 20. Son, S. J., Harris, P. W., Squire, C. J., Baker, E. N., Kent, S. B., Brimble, M. A. (2014). Total Chemical Synthesis of an Orf Virus Protein, ORFV002, an Inhibitor of the Master Gene Regulator NF-κB. Biopolymers (Peptid Science), 102(2):137-144.

21. Wang, G. X., Shang, Y. J., Chen, J. T., Lu, Z. L., Zhang, K. S., Liu, X. T. (2012). Isolation and identification of orf virus in Hubei Province, Advances in Animal Medicine, 033 (011): 37-40.

SEQUENCEAS

| SEQ | | |
|---|---|---|
| ID | | |
| NO: | Description | Sequence |
| 1. | Complete | MKAVLLLALLGAFTNAAPLLSNQRLGSAEEEKFCSTHQGEVHARF |
| | sequence of | WLQMRVGVRHSPLYTPSNMCMMDIEDSTDTEDSTMEKEYTSTATG |
| | the CBP | DADGLNVSVALIGEGVSIPLSYIGLRFNPSLTDGYLYVNVSSRAP |
| | protein | WDQQTLDLSANDGWGIKQVLEKEILAIQIGCDNQKFPEEPTTTQP |
| | (287 aa), | PSPVTTTLSSTTLDPNDENTDTTPTTTGDSVDGKRNPDDFDFSLI |
| | underlined: | VDPRCVTSVNLHFEIKDACMDHKESSPLSLKGEYGDGELVRKEIK |
| | the signal | NVGKDHNMCSLNLSPGH |
| | peptide | |
| 2. | Mature | APLLSNQRLGSAEEEKFCSTHQGEVHARFWLQMRVGVRHSPLYTP |
| | sequence of | SNMCMMDIEDSTDTEDSTMEKEYTSTATGDADGLNVSVALIGEGV |
| | the CBP | SIPLSYIGLRFNPSLTDGYLYVNVSSRAPWDQQTLDLSANDGWGI |
| | protein | KQVLEKEILAIQIGCDNQKFPEEPTTTQPPSPVTTTLSSTTLDPN |
| | (271 aa) | DENTDTTPTTTGDSVDGKRNPDDFDFSLIVDPRCVTSVNLHFEIK |
| | | DACMDHKESSPLSLKGEYGDGELVRKEIKNVGKDHNMCSLNLSPG |
| | | H |
| 3. | Complete sequence of the ORFV112 gene (1162 nt) | 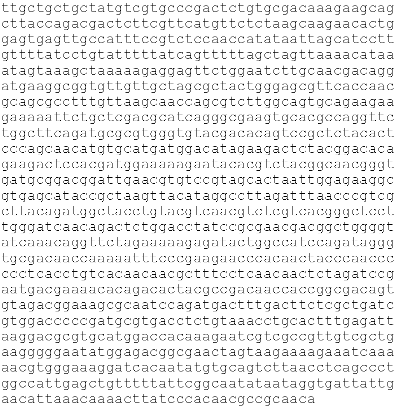 |
| 4. | 5′ end partially deleted ORFV112 gene (624 nt) | 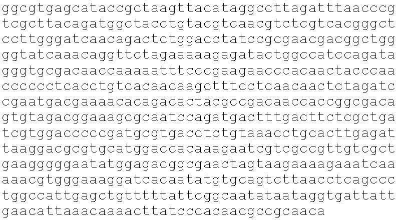 |
| 5. | ORFV112 | catgcccaactgctacttcg |
| | gene- | |
| | specific | |
| | forward | |
| | primer | |
| 6. | ORFV112 | acctgtttgataccccagcc |
| | gene- | |
| | specific | |
| | reverse | |
| | primer | |
| 7 | Forward | gaccatgattacgccaagcttcgtgatttgggaatatgcacc |
| | primer for | |
| | amplifica- | |
| | tion of | |
| | the ORFV112 | |
| | gene′s left | |
| | arm | |
| 8. | Reverse | aaaataaaatttcaatttttataacttcgtatagcatacattata |
| | primer for | cgaagttatgcagcattaactgcgcgctggg |
| | amplifica- | |
| | tion of | |
| | the ORFV112 | |
| | gene′s left | |
| | arm | |
| 9. | Forward | ttttttttggaatataaatagctagcttaattaacttatcccaca |
| | primer for | acgccgcaac |
| | amplifica- | |
| | tion of | |
| | the ORFV112 | |
| | gene′s right | |
| | arm | |
| 10. | Reverse | aaaacgacggccagtgaattcgtatctatcgtcgggaccag |
| | primer for | |
| | amplifica- | |
| | tion of | |
| | the ORFV112 | |
| | gene′s right | |
| | arm | |
| 11. | Forward | aaaaattgaaattttattttttttttttggaatataaataaccgg |
| | primer for | tcgccaccatggtgagcaag |
| | amplifica- | |
| | tion of | |
| | the EGFP | |
| | gene | |
| 12. | Reverse | tatttatattccaaaaaaaaaaaataaaatttcaatttttataac |
| | primer for | ttcgtatagcatacattatacgaagttatacgcgttaagatacat |
| | amplifica- | tgatgagttt |
| | tion of the | |
| | EGFP gene) | |
| 13. | Forward | gaccatgattacgccaagcttcgtgatttgggaatatgcacc |
| | primer for | |
| | amplifica | |
| | tion of the | |
| | linear CBP | |
| | shuttle | |
| | vector | |
| 14. | Reverse | aaaacgacggccagtgaattcgtatctatcgtcgggaccag |
| | primer for | |
| | amplifica- | |
| | tion of the | |
| | linear CBP | |
| | shuttle | |
| | vector | |
| 15. | Forward | gtaagaattcctagagctcgctgatcagcc |
| | primer for | |
| | amplifica | |
| | tion of | |
| | the px330 | |
| | backbone | |
| 16. | Reverse | tggcaccggtccaacctgaaaaaaagt |
| | primer for | |
| | amplifica- | |
| | tion of | |
| | the px330 | |
| | backbone | |
| 17. | Forward | ttggaccggtgccaccatggacaagaagt |
| | primer for | |
| | amplifica- | |
| | tion of | |
| | Cas9 without | |
| | the NLS | |
| | (sequence) | |
| 18. | Reverse | ggctgatcagcgagctctaggaattcttac |
| | primer for | |
| | amplifica- | |
| | tion of | |
| | Cas9 without | |
| | the NLS | |
| | (sequence) | |
| 19. | PCR identi- | gagacgccgccgagcaactt |
| | fication | |
| | primer 1 | |
| | (OVM38F/R)- | |
| | Forward | |
| | primer | |
| 20. | PCR identi- | tgcagcacttcctggacatcg |
| | fication | |
| | primer 1 | |
| | (OVM38F/R) | |
| | Reverse | |
| | primer | |
| 21. | PCR identi- | ggacgatcctgtgcggtag |
| | fication | |
| | primer 2 | |
| | (OVM43F/R)- | |
| | Forward | |
| | primer | |
| 22. | PCR identi- | ttgtcggcgtagtgtctgtg |
| | fication | |
| | primer 2 | |
| | (OVM43F/R)- | |
| | Reverse | |
| | primer | |
| 23. | PCR identi- | gatgactttgacttctcgctgat |
| | fication | |
| | primer 3 | |
| | (OVM44F/R)- | |
| | Forward | |
| | primer | |
| 24. | PCR identi- | cgacagatccatttcccaat |
| | fication | |
| | primer 3 | |
| | (OVM44F/R)- | |
| | Reverse | |
| | primer | |
| 25. | PCR identi- | tgcccaactgctacttcgc |
| | fication | |
| | primer 4 | |
| | (OVM45F/R)- | |
| | Forward | |
| | primer | |
| 26. | PCR identi- | caccttgatgccgttcttct |
| | fication | |
| | primer 4 | |
| | (OVM45F/R)- | |
| | Reverse | |
| | primer | |
| 27. | PCR identi- | agccataccacatttgtagagg |
| | fication | |
| | primer 5 | |
| | (OVM46F/R)- | |
| | Forward | |
| | primer | |
| 28. | PCR identi- | agacgatcaccagcacttcc |
| | fication | |
| | primer 5 | |
| | (OVM46F/R)- | |
| | Reverse | |
| | primer | |
| 29. | PCR identi- | cgtgatttgggaatatgcacc |
| | fication | |
| | primer 6 | |
| | (OVM3F2/ | |
| | 4R2)- | |
| | Forward | |
| | primer | |
| 30. | PCR identi- | gcagcattaactgcgcgctggg |
| | fication | |
| | primer 6 | |
| | (OVM3F2/4 | |
| | R2)- | |
| | Reverse | |
| | primer | |
| 31. | Orf virus | MKVVLLLVLLGALTNAAPVGNQRIDSEEKANFCSTHQNEVYARFR |
| | strain | LQMRVGVRHSPLYVPSNMCMMDIEDSVMDNEYMSAATGDADGVNV |
| | YX | SVALIGEGVSIPLSYIGLGFNPSLADGYLYVNVSSRAPWVQQTQD |
| | AKU76602.1 | LSANGSWGIKQVLEQEMLAIQIGCDNQKFPEEPTTTQPPSPVTTT |
| | | LSSTTLDSNDENADTTPPTTTSASVNRKRNSDDFDFSLLVDPRCV |
| | | TSVDLHVELRDACIDYKEASLLSLKGKYGDGELVKKEIKDVGKDH |
| | | NMCSLNLSPGH |
| 32. | Orf virus | MKAVLLLALLGAFTNAAPLLESQRSNSEEKANFCSTHNNEVYARF |
| | strain | RLQMRVGVRHSPFYTPSNMCMMDIEDSVEDIEESTEKEYASTATG |
| | NA1/11 | EAAGVNVSVALVGEGVSIPFSYIGLGFNPSLEDSYLYVNVSSRAP |
| | AHZ33810.1 | WVKQTSDLSANGGWGIKQVLEKELLAIQIGCDNQKFPEEPTTTPP |
| | | SPVTTTLSSTTPDLNEENTNTTPTTTSASVDRRRNLDDIDFSLLV |
| | | DPRCVTSVDLHVELRDACMDYKQESPLSLKGKYGDGELVKKEIKD |
| | | VGKNYNMCSLNLNPGN |
| 33. | Orf virus | MKAVLLLALLGAFTNAAPLLESQRSNSEEKANFCSTHNDEVYARF |
| | strain | RLQMRVGVRHSPLYTPSNMCMLDIEDSVEDIEESTEKEYASTATG |
| | NZ2 | EAAGVNVSVALVGEGVSIPFSYIGLGFNPSLEDSYLYVNVSSRAP |
| | ABA00630.1 | WVKQTSDLSANGGWGIKQVLEKELLAIQIGCDNQKFPEEPTTTPP |
| | | SPVTTTLSSTTPDLNEENTENTPTTTGASVDRKRNPADDFSLLVD |
| | | PRCVTSVDLHVELRDAICIDYKQESPLSLKGKYGDGELVKKEIKD |
| | | VGKNHNMCSLNLNPGN |
| 34. | Orf virus | MKVVVLLALLGALTNAAPVGNQRLNSKEKEDFCSTHQNEVYARFR |
| | strain | LQMRVGVRHSPLYVPSNMCMMDIEDSVDDIEEDSIIVKEFTSTAT |
| | NA17 | GEAAGVNVSVALVGEGVSIPFSYIGLGFNPSLEDSYLYVNVSSRA |
| | AYM26053.1 | PWVQQTPDLSANDSWGIKQVLEKELLAIQIGCDNQKFPEEPTTTQ |
| | | PPSLVTTTLSSTTLDLNDENTDTTPPTTTSASVNKKRNPDDFDFS |
| | | LLVDPRCVTSVDLHVELRDACIDYKETSQLSLKGEYGDGELIKKE |
| | | IKDVGKDHNMCSLNLNPGN |
| 35. | Orf virus | MKVVLLLVLLGAFTNAAPLVGNQRLDSEEKANFCSTHQNEVYARF |
| | strain | RLQMRVGVRHSPLYTPSNMCMMDIEDSIMDIENSTEKEYTSAATG |
| | OV-SA00 | DANGVNVSVALIGEGVSIPLSYIGLGFNPSLADGYLYVNVSSRAP |
| | AAR98337.1 | WVQQTLDLSANGGWGINQILEKELLAIQIGCDNQKFPEEPTTTQP |
| | | PSPVTTTLSSTTLDLTDENTDTTPLTTTSASVNRKRNPDDFDFSL |
| | | LVDPRCVTSVDLHVELRDACIDYKEASQLSLKGKYGDGELVKKEI |
| | | KDVGKDHNMCSLNLSPGH |
| 36. | Orf virus | MKAAAVLLLALLGAFTNAAPVSNQRLGSEEKEKFCSTHHDEVYAR |
| | strain | FRLQMRVGVRHSPLYVPSNMCMMDIEDSTDDIEESTEKEYTSTAT |
| | OV-IA82 | GEAAGVNVSVALVGEGVKIPFSYIGLGFNPSTDGYLYVNVSSRAP |
| | AAR98207.1 | WVQQTPDLSANSGWGIKQVLEKELLAIQIGCDNQKFPEEPTTTPS |
| | | LVTTTLSPTTTLNPNNENTDTTPTPTGASVDGKRNPDDIDFSLIV |
| | | DPRCVTSVNLHFELKDACMDYKKESPLSLKGKYGDSELVKQEIKD |
| | | VGKNHNMCSLNLSPGH |
| 37. | Orf virus | MKVVLLLVLLGALTNAAPVGNQRIDSEEKANFCSTHQNEVYARFR |
| | strain | LQMRVGVRHSPLYTPSNMCMMNIEDSMMDNEYTSTATGDADGVNV |
| | GO | SVALMGKGVSIPLSYIGLGFNPLLADGYLYVNVSSRAPWVQQTPD |
| | AKU76734.1 | LSANGGWGIKQVLEEEILAIQIGCDNQKFLEEPTTTQPPSLVTTT |
| | | LSSTTLGLTDENTDTTPTTTSASVDRKRNLDDIDFSLLVDTRCVT |
| | | SVNLHFEIKDACMDYKKESPLMMKGGYGDGELVRKEIKYVGTNHT |
| | | MCSLNLSPGY |
| 38. | Orf virus | MKVVVLLALLGALTNAAPVGNQRLNSKEKEDFCLTHPDEVYARFR |
| | strain | LHMRVGVRHSPLYTPSNMCMMNIEDSMMDNEYTSTATGDADGVNV |
| | SJ1 | SVALMGKGVSIPLSYIGLGFNPLLADGYLYVNVSSRAPWVQQTPD |
| | AKU76990.1 | LSANGGWGIKQVLEEEILAIQIGCDNQKFLEEPTTPQPPSLVTTT |
| | | LSSTTLGLTDENTDTTPTTTSASVDRKRNLDDIDFSLLVDTRCVT |
| | | SVNLHFEIKDACMDYKKESPLMMKGGYGDGELVRKEIKYVGTNHT |
| | | MCSLNLSPGY |
| 39. | Orf virus | MKAVLLLALLGAFTNAAPVGNQRLDSGEKEKFCSTHQDEVYARFR |
| | strain | LQMRVGVRHSPFYTPSNMCMMDIEDSVDDIEEDSIIVKEFTSTAT |
| | SY17 | GEADGVNVSVALVGEDVKIPLSYIGLGFNPSTDGYLYVNVSSRAP |
| | AYN61060.1 | WVQQTLDLSANDSWGIKQVLEKELLAIQIGCDNQKFPEEPTTTQP |
| | | PSLVTTTLSSTTPDLNDENTDTTPTTTGASVDRKRNPADFSFSLL |
| | | VDPRCVTSVDLHVELRDACMEYKETSPLSLKGEYGDGELVKKEIK |
| | | DVGKNHNMCSLNLNPGN |
| 40. | Orf virus | MKAVLLLALLGAFTNAAPLLESQRSNSEEKANFCSTHNNEVYARF |
| | strain | RLQMRVGVRHSPFYTPSNMCMMDIEDSVEDIEESTEKEYASTATG |
| | OV-HN3/12 | EAAGVNVSVALVGEGVSIPFSYIGLGFNPSLEDSYLYVNVSSRAP |
| | ASY92409.1 | WVKQTSDLSANGGWGIKQVLEKELLAIQIGCDNQKFPEEPTTTPP |
| | | SPVTTTLSSTTPDLNEENTDTTPTTTSASVDRRRNLDDIDFSLLV |
| | | DPRCVTSVDLHVELRDACMDYKQESPLSLKGKYGDGELVKKEIKD |
| | | VGKNYNMCSLNLNPGN |
| 41. | Orf virus | MKVVVLLALLGALTNAAPVGNQRLNSKEKEDFCSTHPDEVYARFR |
| | strain | LQMRVGVRHSPLYVPSNMCMMDIEDSVDDIEEDSIIVKEFTSTAT |
| | NP | GEAAGVNVSVALVGEGVSIPFSYIGLGFNPSLEDSYLYVNVSSRA |
| | AKU76866.1 | PWVQQTPDLSANDSWGIKQVLEKELLAIQIGCDNQKFPEEPTTTQ |
| | | PPSLVTTTLSSTTLDLNDENTDTTPPTTTSASVNKKRNPDDFDFS |
| | | LLVDPRCVTSVDLHVELRDACIDYKETSQLSLKGEYGDGELIKKE |
| | | IKDVGKDHNMCSLNLNPGN |
| 42. | Orf virus | MKAVLLLALLGAFTNAAPLLSNQRLGSAEEEKFCSTHHDKVYARF |
| | strain | WLQMRVGVRHSSLYAPSNMCMMDIEDSTVDIEGSTETEDSTVEKE |
| | B029 | YTSAATGDANGVNVSVALMGEGVSIPLSYIGLGFNPLLKDGYLYV |
| | AHH34297.1 | NVSSRAPWDQQTLDLSANDGWGIKQVLEKEILAIQIGCDNQKFPE |
| | | EPTTTQPPSPVTTTLSPTTTLNPNNENTDTTPTPTGASVDGKRNP |
| | | DDIDFSLIVDPRCVTSVNLHFEIKDACMDYKQESPLSLKGKYGDG |
| | | ELVKEEIKDVGKNHNMCSLNLSPGH |
| 43. | Orf virus strain YX KP010353.1 | 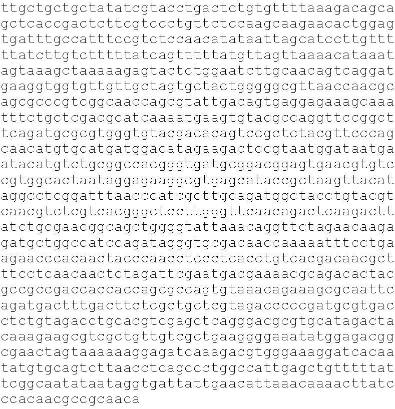 |
| 44. | Orf virus strain NA1/11 KF234407.1 | 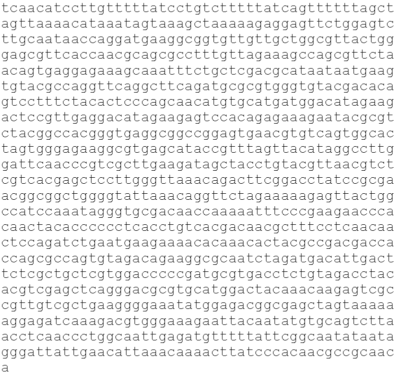 |
| 45. | Orf virus strain NZ2 DQ184476.1 | 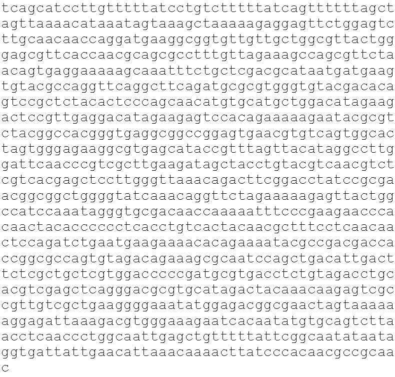 |
| 46. | Orf virus strain NA17 MG674916.2 | 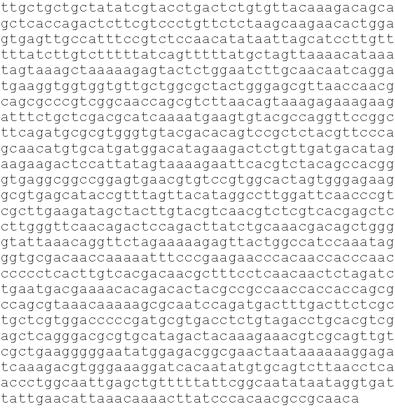 |
| 47. | Orf virus strain OV-SA00 AY386264.1 | 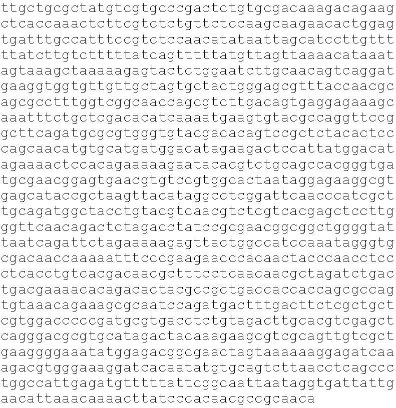 |
| 48. | Orf virus strain OV-IA82 AY386263.1 | 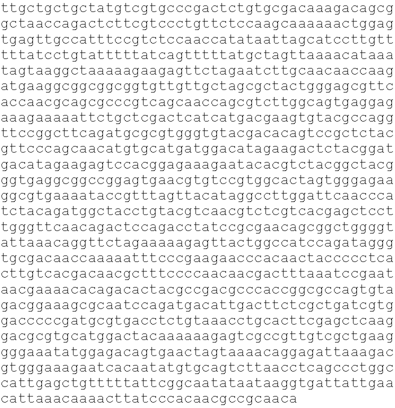 |
| 49. | Orf virus strain GO KP010354.1 | 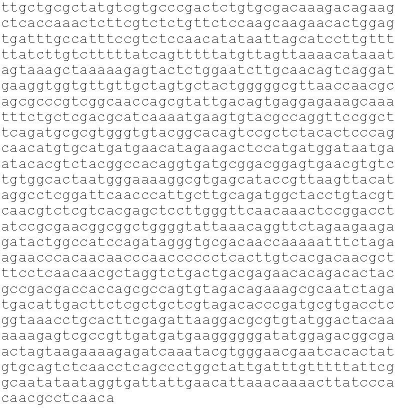 |
| 50. | Orf virus strain SJ1 KP010356.1 | 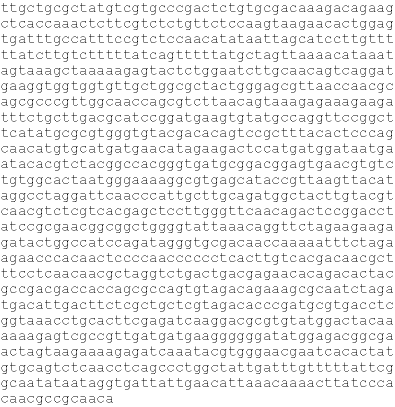 |
| 51. | Orf virus strain SY17 MG712417.1 | 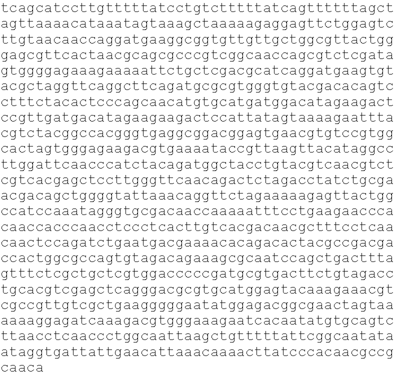 |
| 52. | Orf virus strain OV-HN3/12 KY053526.1 | 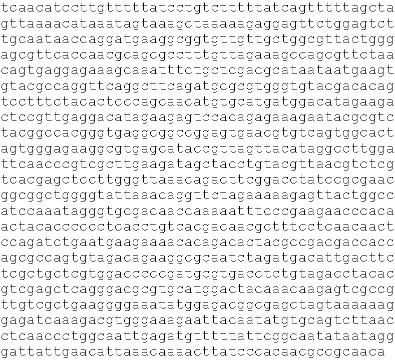 |
| 53. | Orf virus strain NP KP010355.1 | 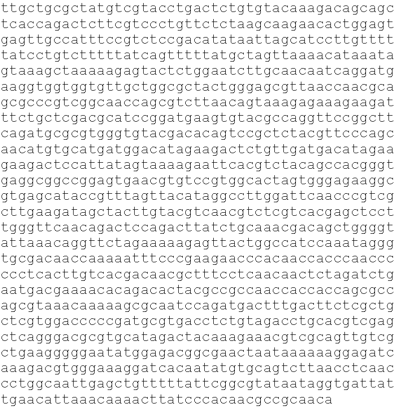 |
| 54. | Orf virus strain B029 KF837136.1 | 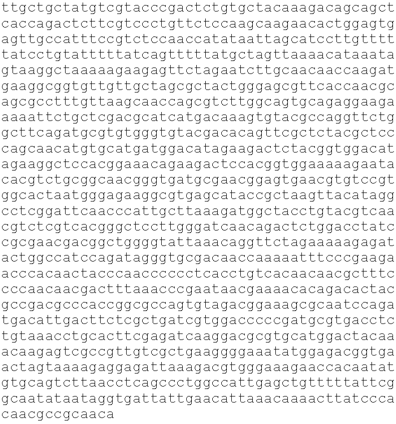 |
| 55. | Forward | cgtgatttgggaatatgcacc |
| | primer | |
| 56. | Reverse | gtatctatcgtcgggaccag |
| | primer | |
| 57. | 5′ truncated | cttatcccacaacgccgcaaca |
| | ORFV112 | |
| | gene | |
| 58. | Full peptide | MSRLQILTSFGQIYAPDEARLREIARDLGICTIKRAFGDMLYGFI |
| | sequence | DFNPVPLTQVNMLMPNCYFAVNGNLLPCTEDFRLRLPATEISAAY |
| | of the | LTKTGRTILCGRDFNIVAPSGFKPSMRLRNLSHVSALVEILELYD |
| | hypothetical | ESGEYQFVLGPSAQLMLRLMEKENVCLFGRGWCIVDLRKLDVAI* |
| | protein 111 | |
| | (179 aa) | |
| 59. | Full DNA sequence of the ORFV111 gene (794 nt) | 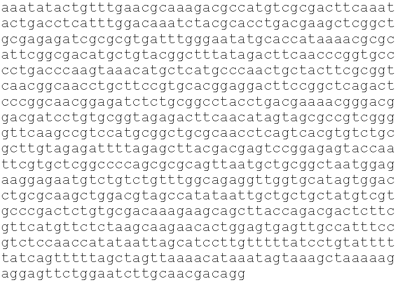 |
| 60. | the 3′ truncated type of the ORFV111 gene (485 nt) | 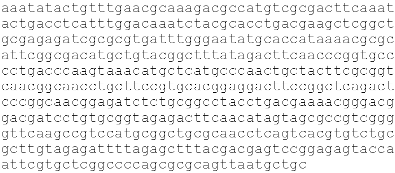 |
| 61. | Orf virus | MSQLQILTSFGQIYAPDEARLREIARDLGICTIKRAFGDMLYGFI |
| | strain | DFNPVPLTQVNMLMSDFYFAVNGNLLPCTEDFRLRLPATDISAAY |
| | YX | LTKTGRTILCGKDFNIVAPSGFKPSMRLRNLSHVSALVEILEFYS |
| | AKU76601.1 | ESGEYQFVLGPSAQFMLRLMEKENVCLFGSGWCIVDLRKLDVTI |
| 62. | Orf virus | MSRLQILTSFGQIYAPDEARLREIARDLGVCTIKRAFGDMLYGFI |
| | strain | DFDPVPLTQVNMLMPNCYFAVNGNLLPCTEDFRLRLPATEISAAY |
| | NA1/11 | LTRTGRTILCGKDFNIVAPSGFKPSMRLRDLSHVSALVEILEIYN |
| | AHZ33809.1 | ESGEYQFVLGPSAQFMLRLMEKENVCLFGSGWCIVDLRKLDVPI |
| 63. | Orf virus | MSRLQILTSFGQIFAPDEARLREIARDLGICTIKRAFGDMLYGFI |
| | strain | DFDPVPLTQVNMLMSNCYFAVNGNLLPCTEDFRLRLPATEISAAY |
| | NZ2 | LTRTGRTILCGKDFNIVAPSGFKPSMRLRDLSHVSALVEILEVYD |
| | ABA00629.1 | ESGEYQFVLGPSAQFMLRLMEKENVCLFGSGWCIVDLRKLDVPI |
| 64. | Orf virus | MSRLQILTSFGQIYAPDEARLREIARDLGICTIKRAFGDMLYGFI |
| | strain | DFNPVPLTQVNMLMSDCYFAVNGNLLPCTEDFRLRLPATEISAAY |
| | NA17 | LTKTGRTILCGKDFNIIAPSGFKPSMRLRNLSHVSALVEILEFYS |
| | AYM26052.1 | ESGEYQFVLGPSAQFMLRLMEKENVCLFGSGWCIVDLRKLDVTI |
| 65. | Orf virus | MSRLQILTSFGQIYAPDEARLREIARDLGICTIKRAFGDMLYGFI |
| | strain | DFNPVPLTQVNMLMSNCYFAVNGNLLPCTEDFRLRLPATEISA |
| | OV-SA00 | AYLTKTGRTILCGKDFNIIAPLGFKPSMRLRNLSHVSALVEILEF |
| | AAR98336.1 | YSESGEYQFVLGPSAQFMLRLMEKENVCLFGSGWCIVDLRKLD |
| | | VTI |
| 66. | Orf virus | MHEGDARITENILFERKDAMSRLQILTSFGQIYAPDEARLREIAR |
| | strain | DLGICTIKRAFGDMLYGFIDFNPVPLTQVNMLMSNCYFAVNGNLL |
| | OV-IA82 | PCTEDFRLRLPATEISAAYLTRTGRTILCGKDFNIVAPSGFKPSM |
| | AAR98206.1 | RLRDLSHVSALVEILELYDESGDYQFVLGPSAQFMLRLMEKENVC |
| | | LFGNGWCIVDLRKLDVTI |
| 67. | Orf virus | MSRLQILTSFGQIYAPDEARLREIARDLGICTIKRAFGDMLYGFI |
| | strain | DFNPVPLTQVNMLMSDCYFAVNGNLLPCTEDFRLRLPATEISAAY |
| | GO | LTKTGRTILCGKDFNIIAPSGFKPSMRLRNLSHVSALVEILEFYS |
| | AKU76733.1 | ESGEYQFVLGPSAQFMLRLMEKENVCLFGSGWCIVDLRKLDVTI |
| 68. | Orf virus | MSQLQILTSFGQIYAPDEARLREIARDLGICTIKRAFGDMLYGFI |
| | strain | DFNPVPLTQVNMLMSNCYFAVNGNLLPCTEDFRLRLPATEISAAY |
| | SJ1 | LTKTGRTILCGKDFNIVAPSGFKPSMRLRNLSHVSALVEILEFYS |
| | AKU76989.1 | ESGEYQFVLGPSAQFMLRLMEKENVCLFGSGWCIVDLRKLDVTI |
| 69. | Orf virus | MSRLQILTSFGQIYAPDEARLREIARDLGICTIKRAFGDMLYGFI |
| | strain | DFNPVPLTQVNMLMPNCYFAVNGNLLPCTEDFRLRLPATEISAAY |
| | SY17 | LTRTGRTILCGKDFNIVAPSGFKPSMRLRDLSHVSALVEILEIYN |
| | AYN61059.1 | ESGEYQFVLGPSAQFMLRLMEKENVCLFGSGWCIVDLRKLDVTI |
| 70. | Orf virus | MSRLQILTSFGQIYAPDEARLREIARDLGVCTIKRAFGDMLYGFI |
| | strain | DFDPVPLTQVNMLMPNCYFAVNGNLLPCTEDFRLRLPATEISAAY |
| | OV-HN3/12 | LTRTGRTILCGKDFNIVAPSGFKPSMRLRDLSHVSALVEILEIYN |
| | ASY92408.1 | ESGEYQFVLGPSAQFMLRLMEKENVCLFGSGWCIVDLRKLDVPI |
| 71. | Orf virus | MSRLQILTSFGQIYAPDEARLREIARDLGICTIKRAFGDMLYGFI |
| | strain | DFNPVPLTQVNMLMSDFYFAVNGNLLPCTEDFRLRLPATDISAAY |
| | NP | LTKTGRTILCGKDFNIVAPSGFKPSMRLRNLSHVSALVEILEFYS |
| | AKU76865.1 | ESGEYQFVLGPSAQFMLRLMEKENVCLFGSGWCIVDLRKLDVTI |
| 72. | Orf virus | MSRLQILTSFGQIYAPDEARLREIARDLGICTIKRAFGDMLYGFI |
| | strain | DFNPVPLTQVNMLMSNCYFAVNGNLLPCTEDFRLRLPATEISAAY |
| | B029 | LTKTGRTILCGKDFNIVAPSGFKPSMRLRDLSHVSALVEILELYD |
| | AIO03573.1 | ESGEYQFVLGPSAQFMLRLMEKENVCLFGNGWCIVDLRKLDVTI |
| 73. | Orf virus strain YX KP010353.1 | 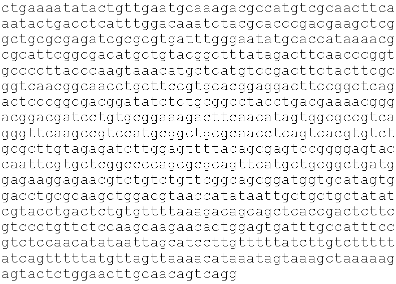 |
| 74. | Orf virus strain NA1/11 KF234407.1 | 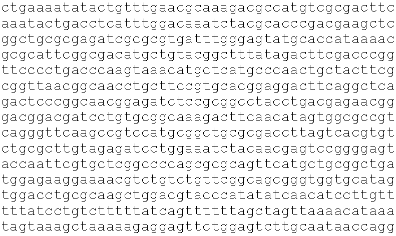 |
| 75. | Orf virus strain NZ2 DQ184476.1 | 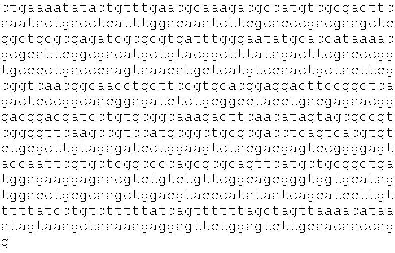 |
| 76. | Orf virus strain NA17 MG674916.2 | 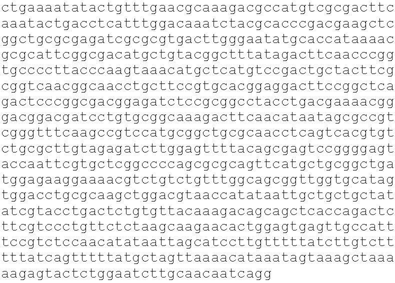 |
| 77. | Orf virus strain OV-SA00 AY386264.1 | 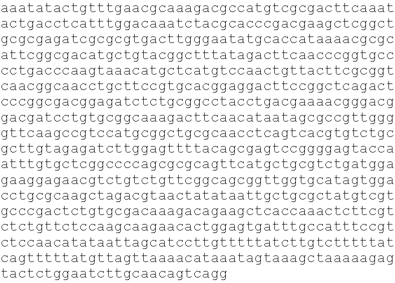 |
| 78. | Orf virus strain OV-IA82 AY386263.1 | 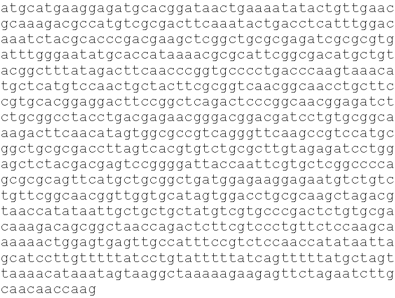 |
| 79. | Orf virus strain GO KP010354.1 | 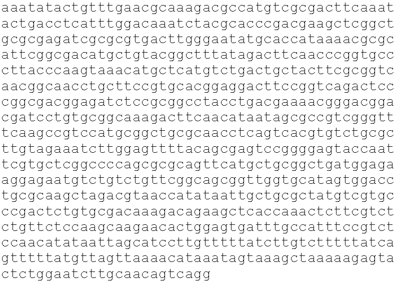 |
| 80. | Orf virus strain SJ1 KP010356.1 | 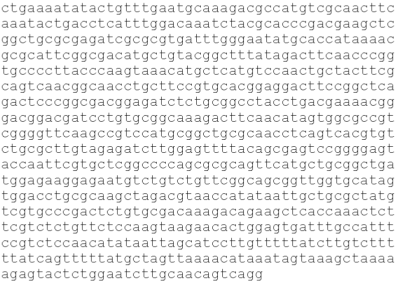 |
| 81. | Orf virus strain SY17 MG712417.1 | 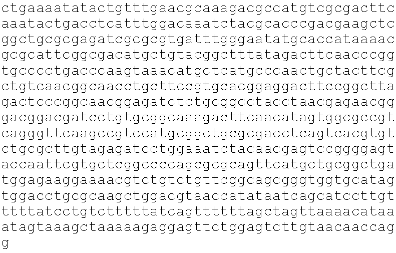 |
| 82. | Orf virus strainOV- HN3/12 KY053526.1 | 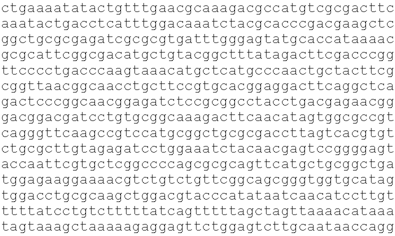 |
| 83. | Orf virus strain NP KP010355.1 | 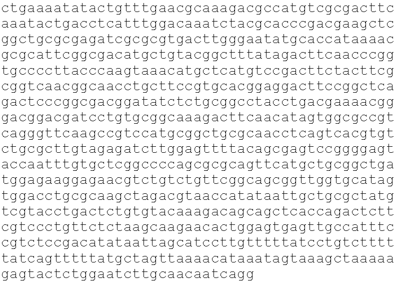 |
| 84. | Orf virus strain B029 KF837136.1 | 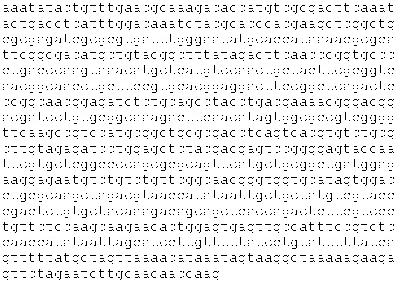 |
| 85. | Amino acid | MSRLQILTSFGQIYAPDEARLREIARDLGICTIKRAFGDMLYGFI |
| | sequence | DFNPVPLTQVNMLMPNCYFAVNGNLLPCTEDFRLRLPATEISAAY |
| | of the | LTKTGRTILCGRDFNIVAPSGFKPSMRLRNLSHVSALVEILELYD |
| | ORFV111 | ESGEYQFVLGPSAQLMLRR |
| | gene of the | |
| | POV-601- | |
| | 1A1 strain | |
| | (mutant) | |
| | (154aa) | |
| 86. | Amino acid | GVSIPLSYIGLRFNPSLTDGYLYVNVSSRAPWDQQTLDLSANDGW |
| | sequence | GIKQVLEKEILAIQIGCDNQKFPEEPTTTQPPSPVTTTLSSTTLD |
| | of the | PNDENTDTTPTTTGDSVDGKRNPDDFDFSLIVDPRCVTSVNLHFE |
| | ORFV112 | IKDACMDHKESSPLSLKGEYGDGELVRKEIKNVGKDHNMCSLNLS |
| | gene of the | PGH |
| | POV-601- | |
| | 1A1 strain | |
| | (mutant) | |
| | (183aa) | |

1. A mutant orf virus, wherein the functionally expressed product of the ORFV112 gene and/or the ORFV111 gene is absent.

2. The virus of claim 1, wherein the absence of the functionally expressed product of the ORFV112 gene is a result of the complete or partial deletion of the ORFV112 gene.

3. The virus of claim 1, wherein the absence of the functionally expressed product of the ORFV111 gene is a result of the complete or partial deletion of the ORFV111 gene.

4. The virus of any one of claims 1-3, wherein the expressed product is protein and/or nucleic acid.

5. A mutant orf virus, characterized by complete or partial deletion of the ORFV112 gene, and/or complete or partial deletion of the ORFV111 gene.

6. A method for modifying an orf virus comprising reducing or suppressing the expression and/or activity of the ORFV112 gene product, and/or reducing or suppressing the expression and/or activity of the ORFV111 gene product.

7. The method of claim 6, wherein the ORFV112 gene is completely or partially deleted.

8. The method of claim 6, wherein the ORFV111 gene is completely or partially deleted.

9. The method of any one of claims 6-8, wherein the expression is transcription and/or translation.

10. A method for modifying an orf virus comprising deleting the ORFV112 gene completely or partially, and/or deleting the ORFV111 gene completely or partially.

11. A virus produced by the method of any one of claims 6-10.

12. The virus of any one of claims 1-5 and 11, wherein the ORFV002 gene (NF-κB inhibitor), the ORFV005 gene (hypothetical protein), and the ORFV007 gene (dUTPase) are completely deleted.

13. The virus of any one of claims 1-5, 11, and 12, wherein the virus has replicating and oncolytic capabilities.

14. An orf virus deposited at China Center for Type Culture Collection (CCTCC) with the accession number V20202.

15. A genome of the virus of any one of claims 1-5 and 11-14.

16. A composition comprising the virus of any one of claims 1-5 and 11-14, and a pharmaceutically acceptable carrier.

17. The composition of claim 16, wherein the composition is in a form of powder, solution, transdermal patch, ointment, or suppository.

18. The composition of claim 16, wherein the composition is administered through intravenous, intratumoral, intramuscular, subcutaneous, rectal, vaginal, or intraperitoneal routes.

19. A use for preparing a drug for cancer treatment with the virus of any one of claims 1-5 and 11-14.

20. The use of claim 19, wherein the cancer is a solid tumor.

21. The use of claim 20, wherein the said solid tumor is cervical cancer, bladder cancer, liver cancer, ovarian cancer, melanoma, colorectal cancer, lung cancer, breast cancer, stomach cancer, uterine cancer, head and neck cancer, thyroid cancer, esophagus cancer, prostate cancer, pancreatic cancer, sarcoma, or a brain tumor.
